SEQUENCE LISTING (22 sequences)

SEQ ID NO: 1 - RNA, 918 nt, Homo sapiens
gggaaaaguagaaagaaagaaagaagagaaaauaaagacaaagagccaccaugugcgugggagcacggagacugggaaggggaccuugcgccgcccugcugcugcugggccugggccuguccaccgugacaggccugcacugcgugggcgacaccuacccuucuaacgauaggugcugucacgagugucgcccaggcaauggcaugguguccaggugcucccgcucucagaacaccgugugccggccuuguggcccaggcuucuauaaugacguggugagcuccaagcccugcaagccuuguacauggugcaaccugcggagcggcuccgagagaaagcagcugugcaccgccacacaggauaccgugugccgguguagagccggcacacagccacuggacucuuacaagccaggaguggauugugcaccuugcccaccuggccacuuuagcccaggcgacaaccaggccuguaagcccuggaccaauugcacacuggcaggcaagcacacccugcagccagcaucuaauucuagcgaugccaucugcgaggacagagauccaccagcaacccagccucaggagacacagggaccuccagccaggccaaucaccgugcagccaacagaggcauggccucggaccucucagggaccaagcacaagacccguggaggugccuggaggaagggcaguggcagcuaucuuggggcucggguugguacugggacugcuuggcccacuugcuaucuugcuggcucuguaucugcugaggcgcgaccagcgccugcccccugaugcacacaagccaccaggaggaggaagcuuccggaccccaauccaggaggagcaggcagacgcacacuccacacuggccaagaucugauuguguaugcguuaauaaaaagaaggaacucgua

SEQ ID NO: 2 - RNA, 903 nt, Mus musculus
gggaaaaguagaaagaaagaaagaagagaaaauaaagacaaagagccaccauguauguguggguucagcagcccacagcccuucugcugcugggacucacacuuggaguuacagcaaggcggcucaacuguguuaaacauaccuaccccaguggucacaagugcugucgugagugccagccaggccaugguauggugagccgcugugaucauaccagggauacucuaugucauccgugugagacuggcuucuacaaugaagcugucaauuaugauaccugcaagcaguguacacagugcaaccaucgaaguggaagugaacucaagcagaauugcacaccuacucaggauacugucugcagauguagaccaggcacccaaccucggcaggacagcggcuacaagcuuggaguugacuguguucccugcccuccuggccacuuuucuccaggcaacaaccaggccugcaagcccuggaccaauuguaccuuaucuggaaagcagacccgccacccagccagugacagcuuggacgcagucugugaggacagaagccuccuggccacacugcucugggagacccagcgcccuacauucaggccaaccacuguccaauccaccacagucuggcccaggacuucugaguugcccucuccacccaccuuggugacuccugagggcccugcauuugcuguucuccuaggccugggccugggccugcuggcucccuugacuguccugcuggccuuguaccugcuccggaaggcuuggagauugccuaacacucccaaaccuuguuggggaaacagcuucaggaccccgauccaggaggaacacacagacgcacacuuuacucuggccaagaucugauuguguaugcguuaauaaaaagaaggaacucgua

SEQ ID NO: 3 - RNA, 50 nt, Artificial Sequence
  Synthetic construct
gggaaaaguagaaagaaagaaagaagagaaaauaaagacaaagagccacc

SEQ ID NO: 4 - RNA, 34 nt, Artificial Sequence
  Synthetic construct
uuguguaugcguuaauaaaaagaaggaacucgua

SEQ ID NO: 5 - RNA, 834 nt, Homo sapiens
augugcgugggagcacggagacugggaaggggaccuugcgccgcccugcugcugcugggccugggccuguccaccgugacaggccugcacugcgugggcgacaccuacccuucuaacgauaggugcugucacgagugucgcccaggcaauggcaugguguccaggugcucccgcucucagaacaccgugugccggccuuguggcccaggcuucuauaaugacguggugagcuccaagcccugcaagccuuguacauggugcaaccugcggagcggcuccgagagaaagcagcugugcaccgccacacaggauaccgugugccgguguagagccggcacacagccacuggacucuuacaagccaggaguggauugugcaccuugcccaccuggccacuuuagcccaggcgacaaccaggccuguaagcccuggaccaauugcacacuggcaggcaagcacacccugcagccagcaucuaauucuagcgaugccaucugcgaggacagagauccaccagcaacccagccucaggagacacagggaccuccagccaggccaaucaccgugcagccaacagaggcauggccucggaccucucagggaccaagcacaagacccguggaggugccuggaggaagggcaguggcagcuaucuuggggcucggguugguacugggacugcuuggcccacuugcuaucuugcuggcucuguaucugcugaggcgcgaccagcgccugcccccugaugcacacaagccaccaggaggaggaagcuuccggaccccaauccaggaggagcaggcagacgcacacuccacacuggccaagaucuga

SEQ ID NO: 6 - RNA, 819 nt, Mus musculus
auguauguguggguucagcagcccacagcccuucugcugcugggacucacacuuggaguuacagcaaggcggcucaacuguguuaaacauaccuaccccaguggucacaagugcugucgugagugccagccaggccaugguauggugagccgcugugaucauaccagggauacucuaugucauccgugugagacuggcuucuacaaugaagcugucaauuaugauaccugcaagcaguguacacagugcaaccaucgaaguggaagugaacucaagcagaauugcacaccuacucaggauacugucugcagauguagaccaggcacccaaccucggcaggacagcggcuacaagcuuggaguugacuguguucccugcccuccuggccacuuuucuccaggcaacaaccaggccugcaagcccuggaccaauuguaccuuaucuggaaagcagacccgccacccagccagugacagcuuggacgcagucugugaggacagaagccuccuggccacacugcucugggagacccagcgcccuacauucaggccaaccacuguccaauccaccacagucuggcccaggacuucugaguugcccucuccacccaccuuggugacuccugagggcccugcauuugcuguucuccuaggccugggccugggccugcuggcucccuugacuguccugcuggccuuguaccugcuccggaaggcuuggagauugccuaacacucccaaaccuuguuggggaaacagcuucaggaccccgauccaggaggaacacacagacgcacacuuuacucuggccaagaucuga

SEQ ID NO: 7 - DNA, 918 bp, Homo sapiens
gggaaaagtagaaagaaagaaagaagagaaaataaagacaaagagccaccatgtgcgtgggagcacggagactgggaaggggaccttgcgccgccctgctgctgctgggcctgggcctgtccaccgtgacaggcctgcactgcgtgggcgacacctacccttctaacgataggtgctgtcacgagtgtcgcccaggcaatggcatggtgtccaggtgctcccgctctcagaacaccgtgtgccggccttgtggcccaggcttctataatgacgtggtgagctccaagccctgcaagccttgtacatggtgcaacctgcggagcggctccgagagaaagcagctgtgcaccgccacacaggataccgtgtgccggtgtagagccggcacacagccactggactcttacaagccaggagtggattgtgcaccttgcccacctggccactttagcccaggcgacaaccaggcctgtaagccctggaccaattgcacactggcaggcaagcacaccctgcagccagcatctaattctagcgatgccatctgcgaggacagagatccaccagcaacccagcctcaggagacacagggacctccagccaggccaatcaccgtgcagccaacagaggcatggcctcggacctctcagggaccaagcacaagacccgtggaggtgcctggaggaagggcagtggcagctatcttggggctcgggttggtactgggactgcttggcccacttgctatcttgctggctctgtatctgctgaggcgcgaccagcgcctgccccctgatgcacacaagccaccaggaggaggaagcttccggaccccaatccaggaggagcaggcagacgcacactccacactggccaagatctgattgtgtatgcgttaataaaaagaaggaactcgta

SEQ ID NO: 8 - DNA, 903 bp, Mus musculus
gggaaaagtagaaagaaagaaagaagagaaaataaagacaaagagccaccatgtatgtgtgggttcagcagcccacagcccttctgctgctgggactcacacttggagttacagcaaggcggctcaactgtgttaaacatacctaccccagtggtcacaagtgctgtcgtgagtgccagccaggccatggtatggtgagccgctgtgatcataccagggatactctatgtcatccgtgtgagactggcttctacaatgaagctgtcaattatgatacctgcaagcagtgtacacagtgcaaccatcgaagtggaagtgaactcaagcagaattgcacacctactcaggatactgtctgcagatgtagaccaggcacccaacctcggcaggacagcggctacaagcttggagttgactgtgttccctgccctcctggccacttttctccaggcaacaaccaggcctgcaagccctggaccaattgtaccttatctggaaagcagacccgccacccagccagtgacagcttggacgcagtctgtgaggacagaagcctcctggccacactgctctgggagacccagcgccctacattcaggccaaccactgtccaatccaccacagtctggcccaggacttctgagttgccctctccacccaccttggtgactcctgagggccctgcatttgctgttctcctaggcctgggcctgggcctgctggctcccttgactgtcctgctggccttgtacctgctccggaaggcttggagattgcctaacactcccaaaccttgttggggaaacagcttcaggaccccgatccaggaggaacacacagacgcacactttactctggccaagatctgattgtgtatgcgttaataaaaagaaggaactcgta

SEQ ID NO: 9 - DNA, 50 bp, Artificial Sequence
  Synthetic construct
gggaaaagtagaaagaaagaaagaagagaaaataaagacaaagagccacc

SEQ ID NO: 10 - DNA, 34 bp, Artificial Sequence
  Synthetic construct
ttgtgtatgcgttaataaaaagaaggaactcgta

SEQ ID NO: 11 - DNA, 834 bp, Homo sapiens
atgtgcgtgggagcacggagactgggaaggggaccttgcgccgccctgctgctgctgggcctgggcctgtccaccgtgacaggcctgcactgcgtgggcgacacctacccttctaacgataggtgctgtcacgagtgtcgcccaggcaatggcatggtgtccaggtgctcccgctctcagaacaccgtgtgccggccttgtggcccaggcttctataatgacgtggtgagctccaagccctgcaagccttgtacatggtgcaacctgcggagcggctccgagagaaagcagctgtgcaccgccacacaggataccgtgtgccggtgtagagccggcacacagccactggactcttacaagccaggagtggattgtgcaccttgcccacctggccactttagcccaggcgacaaccaggcctgtaagccctggaccaattgcacactggcaggcaagcacaccctgcagccagcatctaattctagcgatgccatctgcgaggacagagatccaccagcaacccagcctcaggagacacagggacctccagccaggccaatcaccgtgcagccaacagaggcatggcctcggacctctcagggaccaagcacaagacccgtggaggtgcctggaggaagggcagtggcagctatcttggggctcgggttggtactgggactgcttggcccacttgctatcttgctggctctgtatctgctgaggcgcgaccagcgcctgccccctgatgcacacaagccaccaggaggaggaagcttccggaccccaatccaggaggagcaggcagacgcacactccacactggccaagatctga

SEQ ID NO: 12 - DNA, 819 bp, Mus musculus
atgtatgtgtgggttcagcagcccacagcccttctgctgctgggactcacacttggagttacagcaaggcggctcaactgtgttaaacatacctaccccagtggtcacaagtgctgtcgtgagtgccagccaggccatggtatggtgagccgctgtgatcataccagggatactctatgtcatccgtgtgagactggcttctacaatgaagctgtcaattatgatacctgcaagcagtgtacacagtgcaaccatcgaagtggaagtgaactcaagcagaattgcacacctactcaggatactgtctgcagatgtagaccaggcacccaacctcggcaggacagcggctacaagcttggagttgactgtgttccctgccctcctggccacttttctccaggcaacaaccaggcctgcaagccctggaccaattgtaccttatctggaaagcagacccgccacccagccagtgacagcttggacgcagtctgtgaggacagaagcctcctggccacactgctctgggagacccagcgccctacattcaggccaaccactgtccaatccaccacagtctggcccaggacttctgagttgccctctccacccaccttggtgactcctgagggccctgcatttgctgttctcctaggcctgggcctgggcctgctggctcccttgactgtcctgctggccttgtacctgctccggaaggcttggagattgcctaacactcccaaaccttgttggggaaacagcttcaggaccccgatccaggaggaacacacagacgcacactttactctggccaagatctga

SEQ ID NO: 13 - DNA, 597 bp, Mus musculus
atggaaggggaaggggttcaacccctggatgagaatctggaaaacggatcaaggccaagattcaagtggaagaagacgctaaggctggtggtctctgggatcaagggagcagggatgcttctgtgcttcatctatgtctgcctgcaactctcttcctctccggcaaaggaccctccaatccaaagactcagaggagcagttaccagatgtgaggatgggcaactattcatcagctcatacaagaatgagtatcaaactatggaggtgcagaacaattcggttgtcatcaagtgcgatgggctttatatcatctacctgaagggctcctttttccaggaggtcaagattgaccttcatttccgggaggatcataatcccatctctattccaatgctgaacgatggtcgaaggattgtcttcactgtggtggcctctttggctttcaaagataaagtttacctgactgtaaatgctcctgatactctctgcgaacacctccagataaatgatggggagctgattgttgtccagctaacgcctggatactgtgctcctgaaggatcttaccacagcactgtgaaccaagtaccactgtga

SEQ ID NO: 14 - DNA, 552 bp, Homo sapiens
atggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 15 - DNA, 603 bp, Mus musculus
atgaagccgtacttctgccgtgtctttgtcttctgcttcctaatcagacttttaacaggagaaatcaatggctcggccgatcataggatgttttcatttcacaatggaggtgtacagatttcttgtaaataccctgagactgtccagcagttaaaaatgcgattgttcagagagagagaagtcctctgcgaactcaccaagaccaagggaagcggaaatgcggtgtccatcaagaatccaatgctctgtctatatcatctgtcaaacaacagcgtctcttttttcctaaacaacccagacagctcccagggaagctattacttctgcagcctgtccatttttgacccacctccttttcaagaaaggaaccttagtggaggatatttgcatatttatgaatcccagctctgctgccagctgaagctctggctacccgtagggtgtgcagctttcgttgtggtactcctttttggatgcatacttatcatctggttttcaaaaaagaaatacggatccagtgtgcatgaccctaatagtgaatacatgttcatggcggcagtcaacacaaacaaaaagtctagacttgcaggtgtgacctcataa

SEQ ID NO: 16 - DNA, 600 bp, Homo sapiens
atgaagtcaggcctctggtatttctttctcttctgcttgcgcattaaagttttaacaggagaaatcaatggttctgccaattatgagatgtttatatttcacaacggaggtgtacaaattttatgcaaatatcctgacattgtccagcaatttaaaatgcagttgctgaaaggggggcaaatactctgcgatctcactaagacaaaaggaagtggaaacacagtgtccattaagagtctgaaattctgccattctcagttatccaacaacagtgtctctttttttctatacaacttggaccattctcatgccaactattacttctgcaacctatcaatttttgatcctcctccttttaaagtaactcttacaggaggatatttgcatatttatgaatcacaactttgttgccagctgaagttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctataa

SEQ ID NO: 17 - DNA, 771 bp, Mus musculus
atgggaaacaactgttacaacgtggtggtcattgtgctgctgctagtgggctgtgagaaggtgggagccgtgcagaactcctgtgataactgtcagcctggtactttctgcagaaaatacaatccagtctgcaagagctgccctccaagtaccttctccagcataggtggacagccgaactgtaacatctgcagagtgtgtgcaggctatttcaggttcaagaagttttgctcctctacccacaacgcggagtgtgagtgcattgaaggattccattgcttggggccacagtgcaccagatgtgaaaaggactgcaggcctggccaggagctaacgaagcagggttgcaaaacctgtagcttgggaacatttaatgaccagaacggtactggcgtctgtcgaccctggacgaactgctctctagacggaaggtctgtgcttaagaccgggaccacggagaaggacgtggtgtgtggaccccctgtggtgagcttctctcccagtaccaccatttctgtgactccagagggaggaccaggagggcactccttgcaggtccttaccttgttcctggcgctgacatcggctttgctgctggccctgatcttcattactctcctgttctctgtgctcaaatggatcaggaaaaaattcccccacatattcaagcaaccatttaagaagaccactggagcagctcaagaggaagatgcttgtagctgccgatgtccacaggaagaagaaggaggaggaggaggctatgagctgtga

SEQ ID NO: 18 - DNA, 768 bp, Homo sapiens
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 19 - DNA, 930 bp, Mus musculus
atggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatga

SEQ ID NO: 20 - DNA, 765 bp, Homo sapiens
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 21 - DNA, 687 bp, Mus musculus
atgggggcatgggccatgctgtatggagtctcgatgctctgtgtgctggacctaggtcagccgagtgtagttgaggagcctggctgtggccctggcaaggttcagaacggaagtggcaacaacactcgctgctgcagcctgtatgctccaggcaaggaggactgtccaaaagaaaggtgcatatgtgtcacacctgagtaccactgtggagaccctcagtgcaagatctgcaagcactacccctgccaaccaggccagagggtggagtctcaaggggatattgtgtttggcttccggtgtgttgcctgtgccatgggcaccttctccgcaggtcgtgacggtcactgcagactttggaccaactgttctcagtttggatttctcaccatgttccctgggaacaagacccacaatgctgtgtgcatcccggagccactgcccactgagcaatacggccatttgactgtcatcttcctggtcatggctgcatgcattttcttcctaaccacagtccagctcggcctgcacatatggcagctgaggaggcaacacatgtgtcctcgagagacccagccattcgcggaggtgcagttgtcagctgaggatgcttgcagcttccagttccctgaggaggaacgcggggagcagacagaagaaaagtgtcatctggggggtcggtggccatga

SEQ ID NO: 22 - DNA, 726 bp, Homo sapiens
atggcacagcacggggcgatgggcgcgtttcgggccctgtgcggcctggcgctgctgtgcgcgctcagcctgggtcagcgccccaccgggggtcccgggtgcggccctgggcgcctcctgcttgggacgggaacggacgcgcgctgctgccgggttcacacgacgcgctgctgccgcgattacccgggcgaggagtgctgttccgagtgggactgcatgtgtgtccagcctgaattccactgcggagacccttgctgcacgacctgccggcaccacccttgtcccccaggccagggggtacagtcccaggggaaattcagttttggcttccagtgtatcgactgtgcctcggggaccttctccgggggccacgaaggccactgcaaaccttggacagactgcacccagttcgggtttctcactgtgttccctgggaacaagacccacaacgctgtgtgcgtcccagggtccccgccggcagagccgcttgggtggctgaccgtcgtcctcctggccgtggccgcctgcgtcctcctcctgacctcggcccagcttggactgcacatctggcagctgaggagtcagtgcatgtggccccgagagacccagctgctgctggaggtgccgccgtcgaccgaagacgccagaagctgccagttccccgaggaagagcggggcgagcgatcggcagaggagaaggggcggctgggagacctgtgggtgtga

(19) United States
(12) Patent Application Publication
(10) Pub. No.: US 2022/0152085 A1
(43) Pub. Date: May 19, 2022

(54) COMBINATION IMMUNOREGULATION AND USES THEREOF

(71) Applicant: Ohio State Innovation Foundation, Columbus, OH (US)

(72) Inventors: Yizhou DONG, Dublin, OH (US); Wenqing LI, Columbus, OH (US); Chengxiang ZHANG, Columbus, OH (US)

(21) Appl. No.: 17/442,495

(22) PCT Filed: Mar. 25, 2020
(86) PCT No.: PCT/US2020/024676

§ 371 Date: Sep. 23, 2021

Related U.S. Application Data
(60) Provisional application No. 62/823,184, filed on Mar. 25, 2019.

(51) Int. Cl. A61K 31/7115; A61K 9/127; A61K 39/395; A61P 35/00

(52) U.S. Cl. CPC A61K 31/7115; A61P 35/00; A61K 39/3955; A61K 9/127

(57) ABSTRACT

The present disclosure relates to compositions and methods for regulating the immune system and for treating cancers and other immune disorders.

### CROSS-REFERENCE TO RELATED APPLICATIONS

This application claims the benefit of U.S. Provisional Application No. 62/823,184, filed Mar. 25, 2019, which is expressly incorporated herein by reference.

### STATEMENT REGARDING FEDERALLY SPONSORED RESEARCH

This invention was made with government support under Grant No. R35GM119679 awarded by the National Institutes of Health. The government has certain rights in the invention.

### FIELD

The present disclosure relates to compositions and methods for regulating the immune system and for treating cancers and other immune disorders.

### BACKGROUND

Immunotherapy has become a revolutionary strategy for treating a wide variety of diseases, including various cancers. As key immunoregulatory molecules and signals of immunity are identified and prepared as therapeutic agents, the clinical effectiveness of such therapeutic agents can be tested using well-known cancer models. Immunotherapeutic strategies include administration of vaccines, activated cells, antibodies, cytokines, and chemokines.

The growth and metastasis of tumors depends to a large extent on their capacity to evade host immune surveillance and overcome host defenses. Most tumors express antigens that can be recognized to a variable extent by the host immune system, but in many cases, the immune response is inadequate. Failure to elicit a strong activation of effector T-cells may result from the weak immunogenicity of tumor antigens or inappropriate or absent expression of co-stimulatory molecules by tumor cells. For most T-cells, proliferation and IL-2 production requires a co-stimulator signal during T-cell receptor engagement otherwise, T-cells may enter a functionally unresponsive state.

To date, a number of therapeutic agents and antibodies have been developed as immunotherapeutic agents to regulate the immune system. What is needed are new compositions and methods for stimulating the immune system for treating cancers and other immune disorders.

### SUMMARY

Disclosed herein are compositions and methods which regulate the immune system for treating cancers and other immune disorders. The inventors surprisingly found that when an mRNA encoding a co-stimulatory molecule was administered with an antibody that specifically binds the co-stimulatory molecule, this combination provided improved tumor therapy and overall survival.

In some aspects, disclosed herein is a composition comprising: an antibody, a ligand, or an antigen binding fragment thereof that specifically binds a co-stimulatory molecule; and a nanoparticle comprising an mRNA encoding the co-stimulatory molecule.

In some embodiments, the mRNA encoding the co-stimulatory molecule is encapsulated by the nanoparticle.

In some embodiments, the co-stimulatory molecule is selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM3, TIM4, ICAM1, or LFA3. In some embodiments, the co-stimulatory molecule comprises OX40. In some embodiments, the co-stimulatory molecule comprises 4-1BB (CD137).

In some embodiments, the mRNA encoding the co-stimulatory molecule comprises a heterologous 5′ untranslated region (5′UTR). In some embodiments, the mRNA encoding the co-stimulatory molecule comprises a heterologous 3′ untranslated region (3′UTR).

In some aspects, disclosed herein is a pharmaceutical composition comprising a pharmaceutically acceptable carrier and an effective amount of an antibody, a ligand, or an antigen binding fragment thereof that specifically binds a co-stimulatory molecule and a nanoparticle comprising an mRNA encoding the co-stimulatory molecule.

In some aspects, disclosed herein is a method of stimulating a T cell comprising administering to a subject an effective amount of a composition comprising: an antibody, a ligand, or an antigen binding fragment thereof that specifically binds a co-stimulatory molecule; and a nanoparticle comprising an mRNA encoding the co-stimulatory molecule.

In some embodiments, the subject is a mammal. In some embodiments, the mammal is a human.

In some aspects, disclosed herein is a method of treating a cancer comprising administering to a subject in need thereof an effective amount of an antibody, a ligand, or an antigen binding fragment thereof that specifically binds a co-stimulatory molecule and a nanoparticle comprising an mRNA encoding the co-stimulatory molecule.

In some embodiments, the cancer comprises colorectal cancer or melanoma. In some embodiments, the compositions herein are used to treat both local and metastatic tumors.

In some embodiments, the subject is a mammal. In some embodiments, the mammal is a human.

In some embodiments, the method further comprises administering an additional therapeutic agent. In some embodiments, the additional therapeutic agent comprises an additional immunotherapeutic agent. In some embodiments, the immunotherapeutic agent is selected from an anti-PDL1 antibody, an anti-PD1 antibody, an anti-CTLA4 antibody, or a combination thereof.

### BRIEF DESCRIPTION OF THE DRAWINGS

The accompanying figures, which are incorporated in and constitute a part of this specification, illustrate several aspects described below.

FIG. 1 shows EG.7-OVA cells treated with phosphate buffered saline (PBS) control or nanoparticle (NP)-OX40 mRNA. OX40 positive cells were quantified by the flow cytometry cell sorting analysis.

FIGS. 2A-2B show change of tumor volume in B16 melanoma implanted mice (FIG. 2A) and the mice survival curves (FIG. 2B) following different treatments. NPs+OX40 antibody vs NPs/OX40 mRNA+OX40 antibody: P=0.0010, Log-rank test. PBS vs NPs/OX40 mRNA+OX40 antibody: P=0.0002, Log-rank test. NP represents blank NP; NP/OX40 represents NP comprising OX40 mRNA.

FIG. 3 shows change of tumor volume in a CT26 colon carcinoma mouse tumor model following different treatments. Mice were treated with PBS, nanoparticles (NPs)+anti-OX40 antibody, nanoparticles (NPs)/OX40 mRNA+anti-OX40 antibody and nanoparticles (NPs)/OX40 mRNA+anti-OX40 antibody together. The nanoparticles (NPs)/OX40 mRNA were either injected at 6 hours after anti-OX40 antibody (injection interval: 6 h); or the nanoparticles (NPs)/OX40 mRNA were injected at the same time as the anti-OX40 antibody (injection interval: 0 h). NP represents blank NP; NP/OX40 represents NP comprising OX40 mRNA.

FIGS. 4A-4D show stimulation of T cell mediated cancer immunotherapy. FIG. 4A shows illustration of enhanced antibody immunotherapy via nanoparticles delivering costimulatory receptor mRNA followed by injection of agonistic antibodies to costimulatory receptors (e.g. PL1-OX40 mRNA+anti-OX40 antibody). FIG. 4B shows representative synthetic routes to biomimetic compounds: phospholipid and glycolipid derivatives. i. Et₃N, Toluene, RT. ii. Et₃N, DMF, RT. iii. TFA, CH₂Cl₂, RT. iv. aldehyde, Et₃N, THF, NaBH(OAc)₃. FIGS. 4C-4D show structures of phospholipid derivatives PL1-PL18 (FIG. 4C), and glycolipid derivatives GL1-GL16 (FIG. 4D).

FIGS. 5A-5G show biomimetic phospholipid- and glycolipid-derived nanoparticles for mRNA delivery. FIG. 5A shows luminescence intensity of phospholipid- and glycolipid-derived nanoparticles delivering firefly luciferase (Fluc) mRNA to E.G7 cells. FIG. 5B shows Cryo-TEM image of PL1-OX40 nanoparticles. Scale bar=50 nm. FIG. 5C shows PL1 nanoparticles delivered GFP mRNA to E.G7 cells. FIG. 5D shows PL1-CD137 induced CD137 expression in E.G7 cells. FIG. 5E shows PL1-OX40 induced OX40 expression in EG.7 cells. FIG. 5F shows scheme of GFP expression in B16F10 tumors after a single injection of free GFP mRNA or PL1-GFP. FIG. 5G shows GFP expression in CD4+, CD8+ T cells, after a single intratumoral injection with GFP mRNA (n=4) or PL1-GFP mRNA (n=5) in B16F10 tumors. Data in FIGS. 5A-5E are from n=3 biologically independent samples. All data are presented as mean±S.E.M. Statistical significance in FIGS. 5C, 5D, 5E and 5G were analyzed by the two-tailed Student's t-test. *P<0.05; **P<0.01; ****P<0.0001; n.s., not significant.

FIGS. 6A-6D show regression of B16F10 and A20 tumors after treatment with PL1-CD137 mRNA+anti-CD137 antibody. FIGS. 6A and 6B show that C57BL/6 mice were implanted s.c. with B16F10 melanoma cells. Tumor volume (FIG. 6A) and survival (FIG. 6B) of mice (n=10 per group) after PBS, PL1+anti-CD137 Ab or PL1-CD137+anti-CD137 Ab treatments. PL1-CD137 (10 μg mRNA/mouse), and anti-CD137 Ab (16 μg/mouse). Six i.t. doses were given every other day. FIGS. 6C and 6D show that BALB/c mice were implanted s.c. with A20 lymphoma cells. Tumor volume (FIG. 6C) and survival (FIG. 6D) of mice treated with PBS (n=10), PL1+anti-CD137 Ab (n=12) or PL1-CD137+anti-CD137 Ab (n=12). PL1-CD137 (10 μg mRNA/mouse), and anti-CD137 Ab (16 μg/mouse). Six i.t. doses were given every other day. Data in FIG. 6A and FIG. 6C are presented as mean±S.E.M. Statistical significance in a and c were analyzed by the two-way ANOVA. Statistical significance in FIG. 6B and FIG. 6D were analyzed by the log-rank (Mantel-Cox) test. **P<0.01; ***P<0.001; n.s., not significant.

FIGS. 7A-7D show regression of B16F10 and CT26 tumors after treatment with of PL1-OX40+anti-OX40 Ab. FIGS. 7A-7B show C57BL/6 mice bearing B16F10 melanoma cells. Tumor volumes (FIG. 7A) and survival (FIG. 7B) of mice (n=10 per group) treated with PBS, PL1+anti-OX40 Ab or PL1-OX40+anti-OX40 Ab treatments. PL1-OX40 (10 μg mRNA/mouse) and anti-OX40 Ab (8 μg/mouse). Six i.t. doses were given every other day. FIGS. 7C-7D show that BABL/c mice were implanted subcutaneously with CT26 colon carcinoma cells. Tumor volumes (FIG. 7C) and survival (FIG. 7D) of mice (n=10-11 per group) treated with PBS, PL1+anti-OX40 Ab or PL1-OX40+anti-OX40 Ab. PL1-OX40 (10 μg mRNA/mouse) and anti-OX40 Ab (8 μg/mouse). Six i.t. doses were given every other day. Data in FIG. 7A and FIG. 7C are presented as mean±S.E.M. Statistical significance in FIG. 7A and FIG. 7C were analyzed by the two-way ANOVA. Statistical significance in FIG. 7B and FIG. 7D were analyzed by the log-rank (Mantel-Cox) test. **P<0.01; ***P<0.001; ****P<0.0001.

FIGS. 8A-8H show regression of A20 tumors after treatment with PL1-OX40+anti-OX40 antibody. FIG. 8A shows schematic illustration of the A20 mouse tumor model and the treatment regimen. FIG. 8B shows tumor volumes of individual mice (n=8-10) after six i.t. doses of PBS, PL1-OX40 (10 μg mRNA/mouse), PL1+anti-OX40 Ab (8 μg/mouse), or PL1-OX40+anti-OX40 Ab. FIGS. 8C and 8D show tumor volumes (FIG. 8C) and overall survival (FIG. 8D). FIG. 8E shows rechallenge of mice with complete response (n=6) after treatment with PL1-OX40+anti-OX40 Ab. FIG. 8F shows Treatment plan for evaluation of OX40 expression on the CD8+ T cells after a single i.t. injection with PBS (n=5), OX40 mRNA (n=5), or PL1-OX40 (n=6). FIGS. 8G and 8H show immune cell analysis (CD8+, CD4+ T cells, macrophage, DC) after six i.t. injections with PBS (n=5), PL1+anti-OX40 Ab (n=4), or PL1-OX40+anti-OX40 Ab (n=6), respectively. Data in FIGS. 8C, 8F, and 8H are presented as mean±S.E.M. Statistical significance in FIG. 8C were analyzed by the two-way ANOVA. Statistical significance in FIG. 8D were analyzed by the log-rank (Mantel-Cox) test. Statistical significance in FIG. 8F and FIG. 8H were analyzed by the two-tailed Student's t-test. *P<0.05; **P<0.01; ***P<0.001; ****P<0.0001; n.s., not significant.

FIGS. 9A-9J show antitumor efficacy of PL1-OX40 mRNA+anti-OX40 antibody when combined with surgery or checkpoint inhibitors. FIG. 9A shows schematic illustration of the treatment of PL1-OX40 mRNA+anti-OX40 Ab in combination with surgery (tumors volume <500 mm³). FIG. 9B shows tumor volumes of individual mice (n=10 per group) following six i.t. injections with PBS, anti-OX40 (40 μg) or PL1-OX40 (ψ)+anti-OX40 Ab (n=10). FIGS. 9C and 9D show tumor volumes (FIG. 9C) and survival (FIG. 9D) of mice. FIG. 9E shows rechallange tumor volumes of mice that received PL1-OX40 (ψ)+anti-OX40 (40 μg) followed by surgery to remove residual tumor (n=2) vs. control (n=5). FIG. 9F shows schematic illustration of the treatment of PL1-OX40 mRNA+anti-OX40 Ab in combination with anti-PD-1+anti-CTLA-4 Abs. FIG. 9G shows tumor volumes of individual mice received six doses of PBS (n=10), anti-mouse PD-1+anti-mouse CTLA-4 Abs (n=10), or PL1-OX40 (ψ)+anti-OX40 (40 μg) with anti-PD-1 Ab and anti-CTLA-4 Ab (n=10) every other day. Anti-mouse PD-1+anti-mouse CTLA-4 Abs were injected i.p. every three days for six doses. FIGS. 9H and 9I show tumor volumes (FIG. 9H) and survival (FIG. 9I) of mice. FIG. 9J shows rechallenge tumor volumes of mice that completely responded to the treatment with PL1-OX40 (ψ)+anti-OX40 (40 μg)+anti-PD-1+anti-CTLA-4 antibodies (n=6) vs. control (n=7). Data in FIGS. 9C, 9E, 9H, and 9J are presented as mean±S.E.M. Statistical significance in FIGS. 9C and 9H were analyzed by the two-way ANOVA. Statistical significance in FIGS. 9D and 9I were analyzed by the log-rank (Mantel-Cox) test. ***P<0.001; ****P<0.0001; n.s., not significant.

FIGS. 10A-10F show antitumor efficacy in a lung metastasis mouse model. FIG. 10A shows schematic illustration of lung metastases of B16F10 cells with the treatment of PBS, anti-PD-1+anti-CTLA-4 Abs, or PL1-OX40 mRNA+anti-OX40 Ab+anti-PD-1+anti-CTLA-4 Abs. Mice received i.p. injections of PBS (n=7), i.p. injections of anti-mouse PD-1+anti-mouse CTLA-4 Abs (n=8), or i.v. injections of PL1-OX40 (ψ)+i.p. injections of anti-OX40 (100 μg)+i.p. injections of anti-PD-1 Ab+anti-CTLA-4 Ab (n=9) every three days as shown in the arrows. FIG. 10B shows representative melanoma metastasis in the mouse lungs. FIG. 10C shows lung weights. FIGS. 10D-10F show immune cell analysis of CD8+ T cells, CD4+ T cells, Foxp3+CD4+(Treg) cells in the lungs from different treatments (n=4, 5, 5), respectively. Data in FIGS. 10C-10F are presented as the mean±S.E.M. Statistical significance in FIGS. 10C-10F were analyzed by the two-tailed Student's t-test. *P<0.05; **P<0.01; ***P<0.001; ****P<0.0001; n.s., not significant.

FIG. 11 shows structures of biomimetic lipids: phospholipid and glycolipid derivatives. PL1 and GL1 as representative examples, composed of a biomimetic head (phosphate head or glyco head), an ionizable amino core, and multiple hydrophobic tails.

FIGS. 12A-12C show characterizations of phospholipid and glycolipid derived nanoparticles. FIG. 12A shows particle size (nm) and PDI. FIG. 12B shows Zeta potential (mV). FIG. 12C shows entrapment efficiency of Fluc mRNA. All data are from n=3 biologically independent samples and are presented as mean±S.E.M.

FIG. 13 shows endocytic pathways of the PL1 nanoparticles. E.G7-OVA cells were treated with 5-(N-Methyl-N-isopropyl)amiloride (EIPA), chlorpromazine hydrochloride (CPZ), or methyl-β-cyclodextrin (MβCD). After 0.5 h, cells were treated with PL1-Alexa-Fluor 647-labeled RNA nanoparticles. After 3 h, cells were analyzed by flow cytometry. All data are from n=3 biologically independent samples and are presented as mean±S.E.M. Statistical significance was analyzed by the two-tailed Student's t-test. *P<0.05.

FIGS. 14A-14C show GFP expression in B16F10 tumors after a single injection of GFP mRNA or PL1-GFP mRNA. Macrophages (FIG. 14A) and dendritic cells (FIG. 14B) after a single intratumoral injection with GFP mRNA (n=4) and PL1-GFP mRNA (n=5) in tumor microenvironment. Data in FIG. 14C are presented as mean±S.E.M. Statistical significance was analyzed by the two-tailed Student's t-test. **P<0.01, ***P<0.001.

FIGS. 15A and 15B show tumor growth curves. FIG. 15A shows that C57BL/6 mice were implanted subcutaneously with B16F10 melanoma cells. Tumor volumes of individual mice treated with PBS (n=10), PL1+anti-CD137 Ab (n=10) or PL1-CD137+anti-CD137 Ab (n=10) treatment. PL1-CD137 (10 μg mRNA/mouse) and anti-CD137 Ab (16 μg/mouse). Intratumoral injections every other day for six doses. FIG. 15B shows that BALB/c mice were implanted subcutaneously with A20 lymphoma cells. Tumor volumes of individual mice treated with PBS (n=10), PL1+anti-CD137 Ab (n=12) and PL1-CD137+anti-CD137 Ab (n=12), PL1-CD137 (10 μg mRNA/mouse), and anti-CD137 (16 μg/mouse). Intratumoral injections every other day for six doses.

FIGS. 16A and 16B show tumor growth curves. FIG. 16A shows that C57BL/6 mice were implanted subcutaneously with B16F10 melanoma cells. Tumor volumes of individual animals treated with PBS (n=10), PL1+anti-OX40 Ab (n=10) or PL1-OX40+anti-OX40 Ab (n=10). PL1-OX40 (10 μg mRNA/mouse) and anti-OX40 Ab (8 μg). Intratumoral injection every other day for six doses. FIG. 16B shows that BABL/c mice were implanted subcutaneously with CT26 cells. Tumor volumes of individual animals treated with PBS (n=10), PL1+anti-OX40 Ab (n=11) and PL1-OX40+anti-OX40 Ab (n=11). PL1-OX40 (10 mRNA/mouse) and anti-OX40 Ab (8 μg/mouse). Intratumoral injections every other day for six doses.

FIGS. 17A-17B show analysis of immune cell populations and cytokine levels. FIG. 17A shows OX40 expression on the surface of CD4+ T cells, microphages and dendritic cells after a single intratumoral injection with PBS (n=5), OX40 mRNA (n=5) or PL1-OX40 mRNA (n=6) in tumor microenvironment. FIG. 17B shows mouse plasma cytokine levels after a single intratumoral injection of PBS (n=5), OX40 mRNA (n=4) or PL1-OX40 mRNA (n=6). All data are presented as mean±S.E.M. Statistical significance was analyzed by the two-tailed Student's t-test. **P<0.01; ***P<0.001; ****P<0.0001; n.s., not significant.

FIG. 18 shows effects of CD4+ or CD8+ T cell depletion on immunotherapy of PL1-OX40+anti-OX40 Ab treatment. Tumor volumes of IgG+PL1-OX40+anti-OX40 (n=9), anti-mouse CD8α+PL1-OX40+anti-OX40 (n=9), or anti-mouse CD4+PL1-OX40+anti-OX40 (n=9). All data are presented as mean±S.E.M. Statistical significance was analyzed by the two-way ANOVA. ***P<0.001.

FIG. 19 shows plasma cytokines after six doses of intratumoral treatment. PBS (n=5), PL1+anti-OX40 (n=6) and PL1-OX40+anti-OX40 (n=6). Data are presented as the mean±S.E.M. Statistical significance was analyzed by the two-tailed Student's t-test. n.s., not significant.

FIGS. 20A-20B show antitumor efficacy in a lung metastasis mouse model. 2×10⁵ B16F10 cells were intravenously injected into C57BL/6 mice. Mice received i.p. injections of PBS (n=7), i.p. injections anti-mouse PD-1+anti-mouse CTLA-4 Abs (n=8), or i.v. injections of PL1-OX40 (ψ)+i.p. injections of anti-OX40 (100 μg)+i.p. injections of anti-PD-1 Ab+anti-CTLA-4 Ab (n=9) every three days. FIG. 20A shows mouse body weight. Data are present as the mean±SD. FIG. 20B shows images of melanoma metastasis in the mouse lungs at day 19 after i.v. injection of B16F10 cells.

FIGS. 21A-21D show regression of B16F10 tumors after treatment with i.t. injections of PL1-OX40 and i.p. injections of anti-OX40 antibody. FIG. 21A shows schematic illustration of the B16F10 mouse tumor model and the treatment regimen. FIG. 21B shows tumor volumes of individual mice (n=10 per group) after six i.t. doses of PBS, PL1-OX40(ψ) (10 mRNA/mouse), and two i.p. doses of anti-OX40 Ab (150 μg/mouse). FIGS. 21C-21D show tumor volumes (FIG. 21C) and overall survival (FIG. 21D). Data in FIG. 21C is presented as the mean±S.E.M. Statistical significance in c were analyzed by the two-way ANOVA. Statistical significance in FIG. 21D were analyzed by the log-rank (Mantel-Cox) test. ***P<0.001; ****P<0.0001; n.s., not significant.

FIG. 22 shows gating strategies for flow cytometry analysis. Cells were first gated on FSC/SSC to define single cells. Then, gate CD45 positive cells, CD3 positive cells, CD4/CD8 positive cells and OX40/GFP positive cells. Also, gate CD45 positive cells, CD11b positive cells, CD11c/F4/80 positive cells and OX40/GFP positive cells.

### DETAILED DESCRIPTION

Disclosed herein are compositions and methods which regulate the immune system for treating cancers and other immune disorders.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” or “vector” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. Administration of the therapeutic agents can be carried out at dosages and for periods of time effective for treatment of a subject.

The term “about” as used herein when referring to a measurable value such as an amount, a percentage, and the like, is meant to encompass variations of ±20%, ±10%, ±5%, or ±1% from the measurable value.

A nucleic acid sequence is “heterologous” to a second nucleic acid sequence if it originates from a foreign species, or, if from the same species, is modified by human action from its original form. For example, a heterologous promoter (or heterologous 5′ untranslated region (5′UTR)) operably linked to a coding sequence refers to a coding sequence from a species different from that from which the promoter was derived, or, if from the same species, a coding sequence which is different from naturally occurring allelic variants (for example, the 5′UTR or 3′UTR from a different gene is operably linked to a nucleic acid encoding for a co-stimulatory molecule).

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

As used herein, the term “preventing” a disease, a disorder, or unwanted physiological event in a subject refers to the prevention of a disease, a disorder, or unwanted physiological event or prevention of a symptom of a disease, a disorder, or unwanted physiological event

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

As used herein, the term “controlled-release” or “controlled-release drug delivery” or “extended release” refers to release or administration of a drug from a given dosage form in a controlled fashion in order to achieve the desired pharmacokinetic profile in vivo. An aspect of “controlled” drug delivery is the ability to manipulate the formulation and/or dosage form in order to establish the desired kinetics of drug release.

The phrases “concurrent administration”, “administration in combination”, “simultaneous administration” or “administered simultaneously” as used herein, means that the compounds are administered at the same point in time or immediately following one another.

The term “polypeptide” refers to a compound made up of a single chain of D- or L-amino acids or a mixture of D- and L-amino acids joined by peptide bonds.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv and the like, including hybrid fragments. Thus, fragments of the antibodies that retain the ability to bind their specific antigens are provided. For example, fragments of antibodies which maintain binding activity are included within the meaning of the term “antibody or antigen binding fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “polynucleotide” refers to a single or double stranded polymer composed of nucleotide monomers.

Compositions and Methods

In some aspects, disclosed herein is a composition comprising: an antibody, a ligand, or an antigen binding fragment thereof that specifically binds a co-stimulatory molecule; and a nanoparticle comprising an mRNA encoding the co-stimulatory molecule.

In some aspects, disclosed herein is a composition comprising: an antibody or antigen binding fragment thereof that specifically binds a co-stimulatory molecule; and a nanoparticle comprising an mRNA encoding the co-stimulatory molecule.

In some embodiments, the mRNA encoding the co-stimulatory molecule is encapsulated by the nanoparticle.

In some embodiments, the nanoparticle comprises a phospholipid or a glycolipid. In some embodiments, the nanoparticle comprises a phospholipid. In some embodiments, the nanoparticle comprises a glycolipid. In some embodiments, the phospholipid is selected from the group consisting of PL1-PL18. In some embodiments, the phospholipid is PL1. In some embodiments, the glycolipid is selected from the group consisting of GL1-GL16. In some embodiments, the glycolipid is GL4.

In some embodiments, the co-stimulatory molecule is selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM3, TIM4, ICAM1, or LFA3.

In some embodiments, the co-stimulatory molecule comprises OX40. In some embodiments, the co-stimulatory molecule comprises 4-1BB (CD137). In some embodiments, the co-stimulatory molecule comprises CD30. In some embodiments, the co-stimulatory molecule comprises CD2. In some embodiments, the co-stimulatory molecule comprises B7-H2. In some embodiments, the co-stimulatory molecule comprises B7-1. In some embodiments, the co-stimulatory molecule comprises B7-2. In some embodiments, the co-stimulatory molecule comprises CD70. In some embodiments, the co-stimulatory molecule comprises CD40. In some embodiments, the co-stimulatory molecule comprises 4-1BBL. In some embodiments, the co-stimulatory molecule comprises OX40L.

The sequences for the co-stimulatory molecules include, for example (for human sequences): ICOS (NCBI Reference Sequence: NM_012092.3), CD28 (NCBI Reference Sequence: NM_006139.4), CD27 (NCBI Reference Sequence: NM_001242.4), HVEM (NCBI Reference Sequence: NM_003820.3), LIGHT (NCBI Reference Sequence: NM_003807.4), CD40L (NCBI Reference Sequence: NM_000074.2), 4-1BB (NCBI Reference Sequence: NM_001561.5), OX40 (NCBI Reference Sequence: NM_003327.4), DR3 (NCBI Reference Sequence: NM_148965.1), GITR (NCBI Reference Sequence: NM_004195.3), CD30 (GenBank: M83554.1), SLAM (NCBI Reference Sequence: NM_003037.4), CD2 (NCBI Reference Sequence: NM_001328609.1), CD226 (NCBI Reference Sequence: NM_006566.3), Galectin-9 (GenBank: AB040130.2), TIM1 (GenBank: U02082.1), B7-H2 (NCBI Reference Sequence: NM_015259.5), B7-1 (NCBI Reference Sequence: NM_005191.4), B7-2 (NCBI Reference Sequence: NM_175862.5), CD70 (NCBI Reference Sequence: NM_001252.5), CD40 (NCBI Reference Sequence: NM_001250.5), 4-1BBL (NCBI Reference Sequence: NM_003811.4), OX40L (NCBI Reference Sequence: NM_003326.5), TL1A (NCBI Reference Sequence: NM_005118.4), GITRL (GenBank: AY358868.1), CD30L (NCBI Reference Sequence: NM_001244.3), SLAM (GenBank: U33017.1), CD48 (NCBI Reference Sequence: NM_001778.4), CD58 (NCBI Reference Sequence: NM_001779.3), CD155 (NCBI Reference Sequence: NM_006505.5), CD112 (NCBI Reference Sequence: NM_001042724.2), TIM3 (GenBank: AF450242.1), TIM4 (NCBI Reference Sequence: NM_138379.3), ICAM1 (NCBI Reference Sequence: NM_000201.3).

In some embodiments, the antibody or antigen binding fragment thereof that specifically binds a co-stimulatory molecule is BMS 986178. In some embodiments, the antibody or antigen binding fragment thereof that specifically binds a co-stimulatory molecule is GSK3174998. In some embodiments, the antibody or antigen binding fragment thereof that specifically binds a co-stimulatory molecule is PF-04518600. In some embodiments, the antibody or antigen binding fragment thereof that specifically binds a co-stimulatory molecule is MOXR0916. In some embodiments, the antibody or antigen binding fragment thereof that specifically binds a co-stimulatory molecule is PF-04518600. In some embodiments, the antibody or antigen binding fragment thereof that specifically binds a co-stimulatory molecule is MEDI6383. In some embodiments, the antibody or antigen binding fragment thereof that specifically binds a co-stimulatory molecule is MEDI0562. In some embodiments, the antibody or antigen binding fragment thereof that specifically binds a co-stimulatory molecule is INCAGN01949. In some embodiments, the antibody or antigen binding fragment thereof that specifically binds a co-stimulatory molecule is InVivoPlus anti-mouse OX40 (clone OX-86) (Company: BioXcell, Catalog: BP0031).

Additional antibodies or antigen binding fragments thereof that specifically bind a co-stimulatory molecule can include, for example: for mouse, InVivoPlus anti-mouse 4-1BB (CD137) (clone LOB12.3) (Company: BioXcell, Catalog: BP0169), InVivoPlus anti-mouse CD40 (clone FGK4.5/FGK45) (Company: BioXcell, Catalog: BP0016-2); for human, anti-human OX40, BMS 986178, GSK3174998, PF-04518600, MOXR0916, PF-04518600, MEDI6383, MEDI0562, INCAGN01949; anti-human 4-1BB, Utomilumab, Urelumab; anti-human CD40, CP-870893, APX005M, ADC-1013, JNJ-64457107, SEA-CD40, R07009789.

In some embodiments, the mRNA encoding the co-stimulatory molecule comprises a heterologous 5′ untranslated region (5′UTR). In some embodiments, the mRNA encoding the co-stimulatory molecule comprises a heterologous 3′ untranslated region (3′UTR).

In some embodiments, the nucleic acids (for example, the mRNA encoding the co-stimulatory molecule) disclosed herein comprise at least one chemically modified nucleotide. In some embodiments, the at least one chemically modified nucleotide comprises a chemically modified nucleobase, a chemically modified ribose, a chemically modified phosphodiester linkage, or a combination thereof.

In one embodiment, the at least one chemically modified nucleotide is a chemically modified nucleobase.

In one embodiment, the chemically modified nucleobase is selected from 5-formylcytidine (5fC), 5-methylcytidine (5meC), 5-methoxycytidine (5moC), 5-hydroxycytidine (5hoC), 5-hydroxymethylcytidine (5hmC), 5-formyluridine (5fU), 5-methyluridine (5-meU), 5-methoxyuridine (5moU), 5-carboxymethylesteruridine (5camU), pseudouridine (T), N′-methylpseudouridine (me^{I}Ψ), N⁶-methyladenosine (me⁶A), or thienoguanosine (^{th}G).

In some embodiments, the chemically modified nucleobase is 5-methoxyuridine (5moU). In some embodiments, the chemically modified nucleobase is pseudouridine (Ψ). In some embodiments, the chemically modified nucleobase is N¹-methylpseudouridine (me^{I}Ψ).

The structures of these modified nucleobases are shown below:

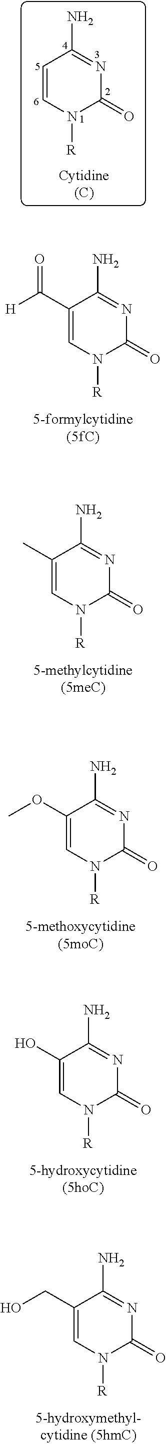

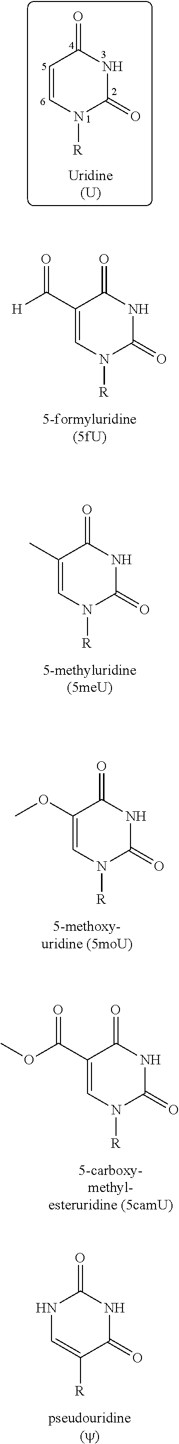

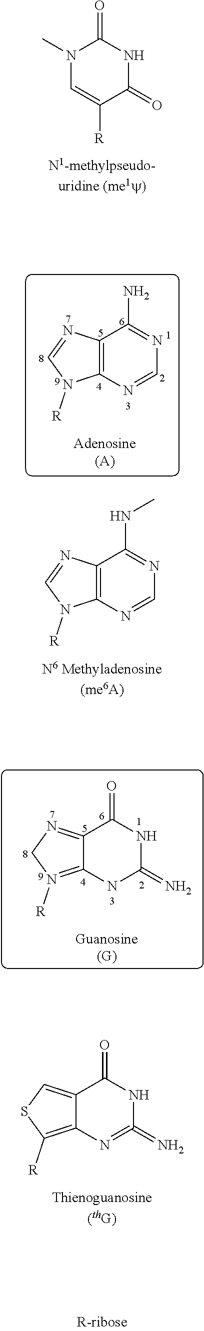

In one embodiment, the at least one chemically modified nucleotide is a chemically modified ribose.

In one embodiment, the chemically modified ribose is selected from 2′-O-methyl (2′-0-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-O-ME), 2′-O-Allyl, 2′-O-Ethylamine, 2′-O-Cyanoethyl, Locked nucleic acid (LAN), Methylene-cLAN, N-MeO-amino BNA, or N-MeO-aminooxy BNA. In one embodiment, the chemically modified ribose is 2′-O-methyl (2′-O-Me). In one embodiment, the chemically modified ribose is 2′-Fluoro (2′-F).

The structures of these modified riboses are shown below:

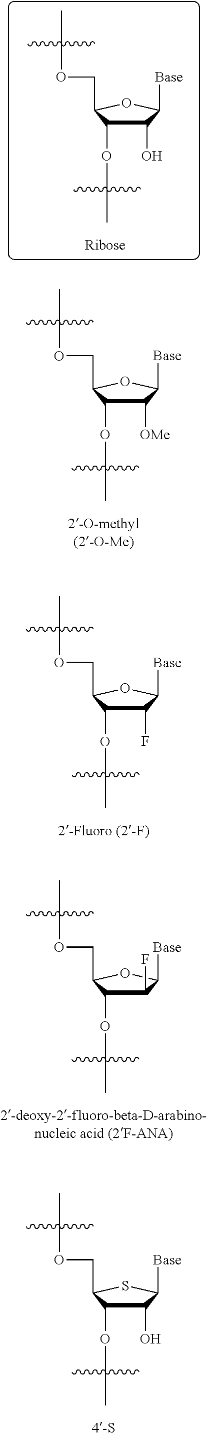

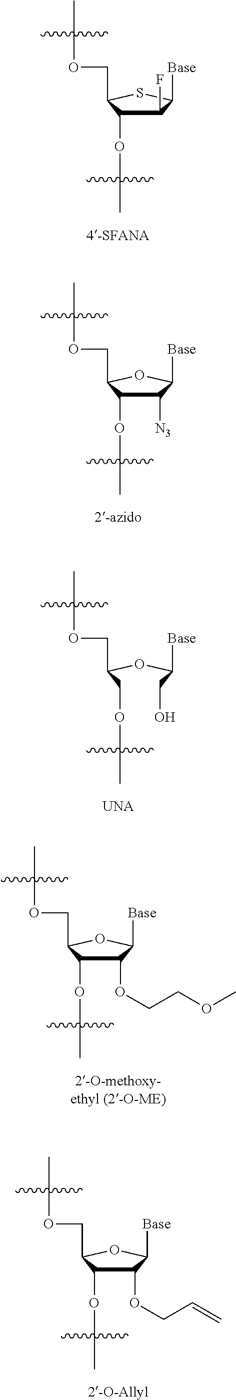

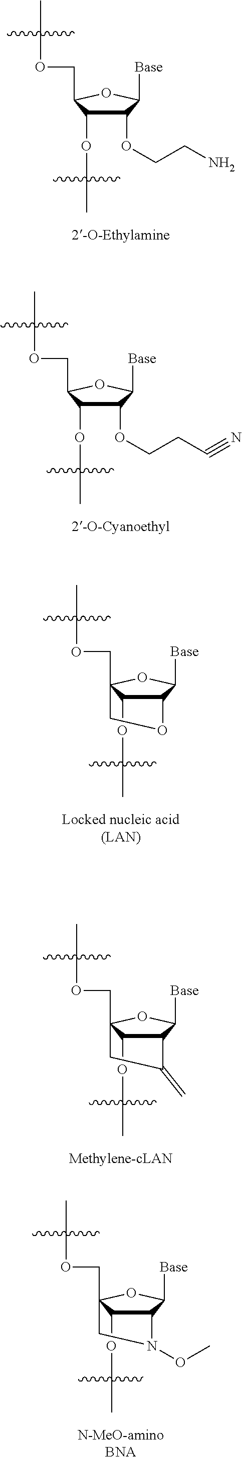

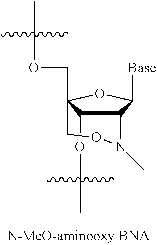

In one embodiment, the at least one chemically modified nucleotide is a chemically modified phosphodiester linkage.

In one embodiment, the chemically modified phosphodiester linkage is selected from phosphorothioate (PS), boranophosphate, phosphodithioate (PS2), 3′,5′-amide, N3′-phosphoramidate (NP), Phosphodiester (PO), or 2′,5′-phosphodiester (2′,5′-PO). In one embodiment, the chemically modified phosphodiester linkage is phosphorothioate.

The structures of these modified phosphodiester linkages are shown below:

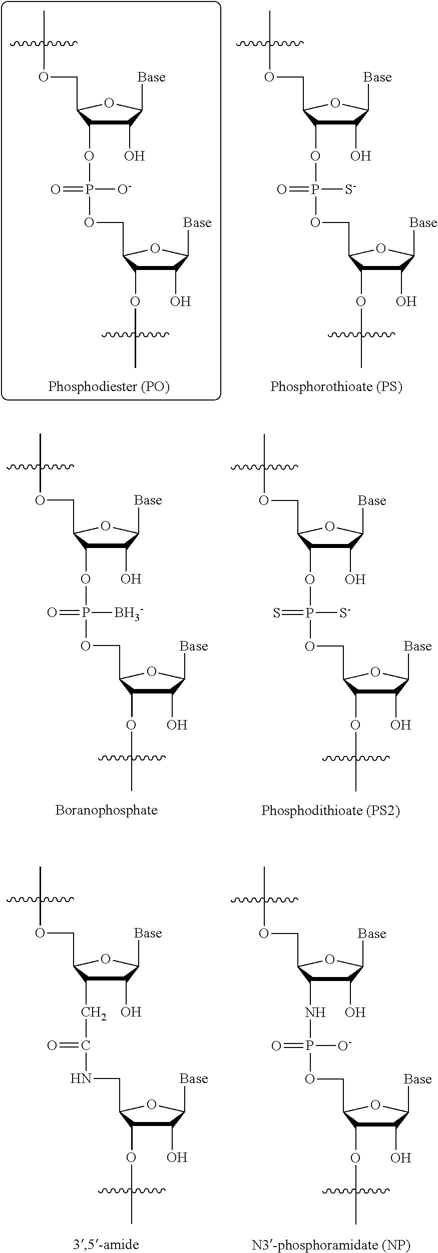

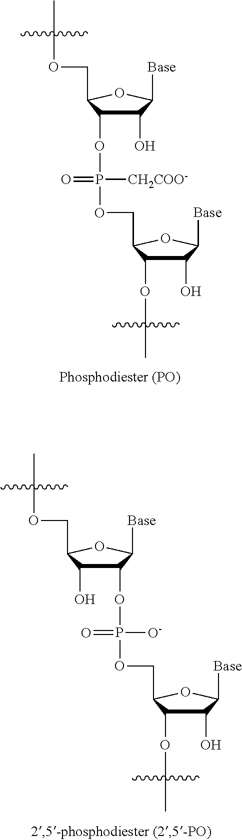

In some embodiments, the composition further comprises an immunotherapeutic agent. In some embodiments, the immunotherapeutic agent is selected from an anti-PDL1 antibody, an anti-PD1 antibody, an anti-CTLA4 antibody, or a combination thereof.

In some aspects, disclosed herein is a pharmaceutical composition comprising a pharmaceutically acceptable carrier and an effective amount of an antibody, a ligand, or an antigen binding fragment thereof that specifically binds a co-stimulatory molecule and a nanoparticle comprising an mRNA encoding the co-stimulatory molecule.

In some aspects, disclosed herein is a pharmaceutical composition comprising a pharmaceutically acceptable carrier and an effective amount of an antibody or antigen binding fragment thereof that specifically binds a co-stimulatory molecule and a nanoparticle comprising an mRNA encoding the co-stimulatory molecule.

In some aspects, disclosed herein is a method of treating a cancer comprising administering to a subject in need thereof an effective amount of an antibody, a ligand, or an antigen binding fragment thereof that specifically binds a co-stimulatory molecule and a nanoparticle comprising an mRNA encoding the co-stimulatory molecule.

In some aspects, disclosed herein is a method of treating a cancer comprising administering to a subject in need thereof an effective amount of an antibody or antigen binding fragment thereof that specifically binds a co-stimulatory molecule and a nanoparticle comprising an mRNA encoding the co-stimulatory molecule.

In some embodiments, the mRNA encoding the co-stimulatory molecule is encapsulated by the nanoparticle.

The nanoparticle used can be any nanoparticle useful for the delivery of nucleic acids. In some embodiments, the nanoparticle comprises a lipid-like nanoparticle. See, for example, WO WO/2016/187531A1, WO/2017/176974, WO/2019/027999, or Li, B et al. An Orthogonal array optimization of lipid-like nanoparticles for mRNA delivery in vivo. Nano Lett. 2015, 15, 8099-8107; which are incorporated herein by reference. In some embodiments, the nanoparticle (or delivery agent) can comprise a lipid bilayer or liposome. In some embodiments, the nanoparticle can comprise a polymer, for example, a biodegradable polymer. Polymers can include, for example, both biostable and biodegradable polymers, such as microcrystalline cellulose, hydroxypropyl cellulose, hydroxypropyl methylcellulose, polyalkylene oxides such as polyethylene oxide (PEG), polyanhydrides, poly(ester anhydrides), polyhydroxy acids such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

In some embodiments, the co-stimulatory molecule is selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM3, TIM4, ICAM1, or LFA3. In some embodiments, the co-stimulatory molecule comprises OX40. In some embodiments, the co-stimulatory molecule comprises 4-1BB (CD137).

In some embodiments, the mRNA encoding the co-stimulatory molecule is isolated. In some embodiments, the mRNA encoding the co-stimulatory molecule is recombinant. In some embodiments, the antibody or antigen binding fragment thereof is isolated. In some embodiments, the antibody or antigen binding fragment thereof is recombinant. In some embodiments, the antibody is a monoclonal antibody.

In some embodiments, the cancer comprises melanoma, colorectal cancer, lung cancer, colon cancer, or lymphoma. In some embodiments, the cancer comprises colorectal cancer or melanoma. In some embodiments, the cancer is colorectal cancer. In some embodiments, the cancer is melanoma. In some embodiments, the composition herein are used to treat both local and metastatic tumors.

In some embodiments, the compositions and methods described herein are useful for treating or preventing metastasis or recurrence of a cancer. In some embodiments, the compositions and methods described herein are useful for the prevention of recurrence of excised solid tumors. In some embodiments, the compositions and methods described herein are useful for the prevention of metastasis of excised solid tumors.

In one aspect, the methods described herein are used to treat cancer, for example, melanoma, lung cancer (including lung adenocarcinoma, basal cell carcinoma, squamous cell carcinoma, large cell carcinoma, bronchioloalveolar carcinoma, bronchogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcinoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas, leiomyosarcomas, mixed mullerian tumors); glioma, glioblastoma, medulloblastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors including sarcomas, fibrosarcomas, haemangioma, angiomatosi s, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, and cancers of the vagina among others.

In some embodiments, the compositions and methods described herein are useful in treating or preventing a cancer. In some cases, the cancer is a circulating cancer cell (circulating tumor cell). In some cases, the cancer is a metastatic cancer cell.

In some embodiments, the subject is a mammal. In some embodiments, the mammal is a human.

In some embodiments, the antibody or antigen binding fragment thereof and the nanoparticle are administered by intramuscularly injection or systematically.

In some embodiments, the method further comprises administering an additional therapeutic agent. In some embodiments, the additional therapeutic agent comprises an additional immunotherapeutic agent. In some embodiments, the immunotherapeutic agent is selected from an anti-PDL1 antibody, an anti-PD1 antibody, an anti-CTLA4 antibody, or a combination thereof.

In one embodiment, the immunotherapeutic agent is an anti-PDL1 antibody. In one embodiment, the anti-PDL1 antibody is selected from atezolizumab, durvalumab, or avelumab. In one embodiment, the anti-PDL1 antibody is atezolizumab (MPDL3280A)(Roche). In one embodiment, the anti-PDL1 antibody is durvalumab (MEDI4736). In one embodiment, the anti-PDL1 antibody is avelumab (MS0010718C).

In one embodiment, the immunotherapeutic agent is a programmed death protein 1 (PD-1) inhibitor or programmed death protein ligand 1 or 2 inhibitor. PD-1 inhibitors are known in the art, and include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech).

In one embodiment, the immunotherapeutic agent is an anti-PD1 antibody. In one embodiment, the anti-PD1 antibody is nivolumab. In one embodiment, the anti-PD1 antibody is pembrolizumab.

In one embodiment, the immunotherapeutic agent is an anti-CTLA4 antibody. In one embodiment, the anti-CTLA4 antibody is ipilimumab.

In some embodiments, the additional therapeutic agent is an anti-neoplastic agent. For example, the anti-neoplastic agent can be selected from the group consisting of Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPDX, Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Interferon Alfa-2b, Recombinant, Intron A (Recombinant Interferon Alfa-2b), Iodine I 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perj eta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and Iodine I 131 Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate).

In some embodiments, the co-stimulatory molecule is ICOS. In some embodiments, the co-stimulatory molecule is CD28. In some embodiments, the co-stimulatory molecule is CD27. In some embodiments, the co-stimulatory molecule is HVEM. In some embodiments, the co-stimulatory molecule is LIGHT. In some embodiments, the co-stimulatory molecule is CD40L. In some embodiments, the co-stimulatory molecule is 4-1BB. In some embodiments, the co-stimulatory molecule is DR3. In some embodiments, the co-stimulatory molecule is GITR. In some embodiments, the co-stimulatory molecule is CD30. In some embodiments, the co-stimulatory molecule is SLAM. In some embodiments, the co-stimulatory molecule is CD2. In some embodiments, the co-stimulatory molecule is CD226. In some embodiments, the co-stimulatory molecule is Galectin9. In some embodiments, the co-stimulatory molecule is TIM1. In some embodiments, the co-stimulatory molecule is LFA1. In some embodiments, the co-stimulatory molecule is B7-H2. In some embodiments, the co-stimulatory molecule is B7-1. In some embodiments, the co-stimulatory molecule is B7-2. In some embodiments, the co-stimulatory molecule is CD70. In some embodiments, the co-stimulatory molecule is LIGHT. In some embodiments, the co-stimulatory molecule is HVEM. In some embodiments, the co-stimulatory molecule is CD40. In some embodiments, the co-stimulatory molecule is 4-1BBL. In some embodiments, the co-stimulatory molecule is OX40L. In some embodiments, the co-stimulatory molecule is TL1A. In some embodiments, the co-stimulatory molecule is GITRL. In some embodiments, the co-stimulatory molecule is CD30L. In some embodiments, the co-stimulatory molecule is SLAM. In some embodiments, the co-stimulatory molecule is CD48. In some embodiments, the co-stimulatory molecule is CD58. In some embodiments, the co-stimulatory molecule is CD155. In some embodiments, the co-stimulatory molecule is CD112. In some embodiments, the co-stimulatory molecule is CD80. In some embodiments, the co-stimulatory molecule is CD86. In some embodiments, the co-stimulatory molecule is ICOSL. In some embodiments, the co-stimulatory molecule is TIM3. In some embodiments, the co-stimulatory molecule is TIM4. In some embodiments, the co-stimulatory molecule is ICAM1. In some embodiments, the co-stimulatory molecule is LFA3.

In some embodiments, the co-stimulatory molecule is OX40. In some embodiments, the OX40 co-stimulatory molecule comprises the mRNA sequence SEQ ID NO: 1. In some embodiments, the OX40 co-stimulatory molecule comprises the mRNA sequence SEQ ID NO: 2. In some embodiments, the OX40 co-stimulatory molecule comprises the mRNA sequence SEQ ID NO: 5. In some embodiments, the co-stimulatory molecule is OX40. In some embodiments, the OX40 co-stimulatory molecule comprises the mRNA sequence SEQ ID NO: 6.

In some embodiments, the OX40 co-stimulatory molecule comprises a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1, or a variant or a fragment thereof. In some embodiments, the OX40 co-stimulatory molecule comprises a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 2, or a variant or a fragment thereof. In some embodiments, the OX40 co-stimulatory molecule comprises a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 5, or a variant or a fragment thereof. In some embodiments, the OX40 co-stimulatory molecule comprises a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 6, or a variant or a fragment thereof.

In some embodiments, the co-stimulatory molecule is encoded by a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence of a co-stimulatory molecule selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM3, TIM4, ICAM1, LFA3, or a variant or a fragment thereof.

In some embodiments, the mRNA encoding the co-stimulatory molecule comprises a modified 5′ untranslated region (5′UTR). In some embodiments, the mRNA encoding the co-stimulatory molecule comprises a modified 3′ untranslated region (3′UTR). For example, a modified sequence could include insertions, deletions, or nucleotide substitutions.

In some embodiments, the mRNA encoding the co-stimulatory molecule comprises a heterologous 5′ untranslated region (5′UTR) comprising the mRNA sequence SEQ ID NO: 3. In some embodiments, the mRNA encoding the co-stimulatory molecule comprises a heterologous 3′ untranslated region (3′UTR) comprising the mRNA sequence SEQ ID NO: 4. In some embodiments, the mRNA encoding the co-stimulatory molecule comprises a heterologous 5′ untranslated region (5′UTR) comprising a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 3, or a variant or a fragment thereof. In some embodiments, the mRNA encoding the co-stimulatory molecule comprises a heterologous 3′ untranslated region (3′UTR) comprising a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 4, or a variant or a fragment thereof.

In some aspects, disclosed herein is a method of stimulating a T cell comprising administering to a subject an effective amount of a composition comprising: an antibody, a ligand, or an antigen binding fragment thereof that specifically binds a co-stimulatory molecule; and a nanoparticle comprising an mRNA encoding the co-stimulatory molecule.

In some aspects, disclosed herein is a method of stimulating a T cell comprising administering to a subject an effective amount of a composition comprising: an antibody or antigen binding fragment thereof that specifically binds a co-stimulatory molecule; and a nanoparticle comprising an mRNA encoding the co-stimulatory molecule.

In some embodiments, the antigen binding fragment that specifically binds a co-stimulatory molecule comprises an OX40 ligand or a functional fragment thereof that binds to OX40. In some embodiments, the OX40 ligand is encoded by a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 13 or 14.

In some embodiments, the antigen binding fragment that specifically binds a co-stimulatory molecule comprises an ICOS ligand or a functional fragment thereof that binds to ICOS. In some embodiments, the ICOS ligand is encoded by a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 15 or 16.

In some embodiments, the antigen binding fragment that specifically binds a co-stimulatory molecule comprises a CD137 ligand or a functional fragment thereof that binds to CD137. In some embodiments, the CD137 ligand is encoded by a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 19 or 20. In some embodiments, the subject is a mammal. In some embodiments, the mammal is a human. In some embodiments, the T-cells comprise CD4+ T-cells, CD8+ T-cells, or combinations thereof. In some embodiments, the T-cells comprise CD8+ T-cells. CD8+ T-cells are also referred to as cytotoxic T-cells and can function to kill specifically recognized cells (e.g., tumor cells).

In some embodiments, the antibody or antigen binding fragment thereof that specifically binds a co-stimulatory molecule and the nanoparticle comprising an mRNA encoding the co-stimulatory molecule are administered concurrently (simultaneously or immediately thereafter). In some embodiments, the antibody or antigen binding fragment thereof that specifically binds a co-stimulatory molecule and the nanoparticle comprising an mRNA encoding the co-stimulatory molecule are administered sequentially.

Also disclosed herein are methods of treating a disease or a condition such as an inflammation disorder (including an autoimmune disease) or lymphoid proliferative diseases, comprising administering to a subject in need thereof an effective amount of an antibody, a ligand, or an antigen binding fragment thereof that specifically binds a co-stimulatory molecule and a nanoparticle comprising an mRNA encoding the co-stimulatory molecule.

Further disclosed herein are methods of treating a disease or a condition such as an inflammation disorder (including an autoimmune disease) or lymphoid proliferative diseases, comprising administering to a subject in need thereof an effective amount of an antibody or antigen binding fragment thereof that specifically binds a co-stimulatory molecule and a nanoparticle comprising an mRNA encoding the co-stimulatory molecule.

In one embodiment, provided herein is a method of treating an inflammation disorder, including autoimmune diseases in a subject. The method comprises administering to said subject a therapeutically effective amount of a compound, a combination of compounds, or a composition provided herein, or a pharmaceutically acceptable form thereof, or a pharmaceutical composition as provided herein. Examples of autoimmune diseases include but are not limited to acute disseminated encephalomyelitis (ADEM), Addison's disease, antiphospholipid antibody syndrome (APS), aplastic anemia, autoimmune hepatitis, autoimmune skin disease, coeliac disease, Crohn's disease, Diabetes mellitus (type 1), Goodpasture's syndrome, Graves' disease, Guillain-Barr-syndrome (GBS), Hashimoto's disease, lupus erythematosus, multiple sclerosis, myasthenia gravis, opsoclonus myoclonus syndrome (OMS), optic neuritis, Ord's thyroiditis, oemphigus, polyarthritis, primary biliary cirrhosis, psoriasis, rheumatoid arthritis, Reiter's syndrome, Takayasu's arteritis, temporal arteritis (also known as “giant cell arteritis”), warm autoimmune hemolytic anemia, Wegener's granulomatosis, alopecia universalis (e.g., inflammatory alopecia), Chagas disease, chronic fatigue syndrome, dysautonomia, endometriosis, hidradenitis suppurativa, interstitial cystitis, neuromyotonia, sarcoidosis, scleroderma, ulcerative colitis, vitiligo, and vulvodynia. Other disorders include bone-resorption disorders and thrombosis.

Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation.

Exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gout flare, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, type 2 diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, polymyalgia rheumatic, reperfusion injury, regional enteritis, rheumatic fever, systemic lupus erythematosus, scleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds can also be useful in treating inflammation associated with trauma and non-inflammatory myalgia.

Immune disorders, such as auto-immune disorders include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), relapsing polychondritis (e.g., atrophic polychondritis and systemic polychondromalacia), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Nanoparticle (NP)-OX40 mRNA Induced Increased Expression Levels of OX40

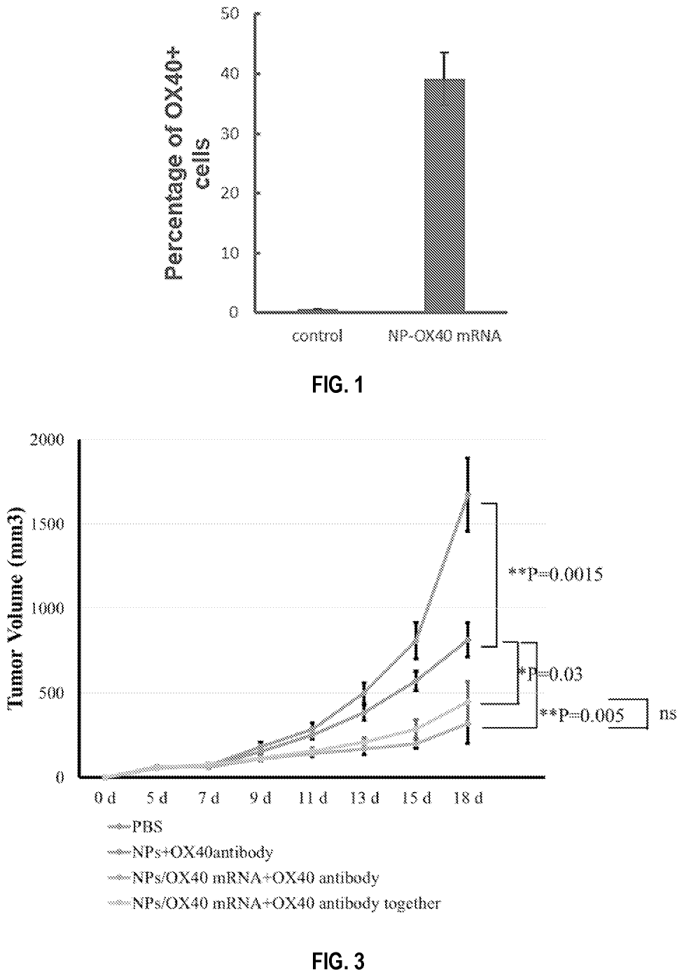

OX40 expression was characterized in EG.7-OVA cells. Nanoparticle (NP)-OX40 mRNA induced much higher OX40 expression compared to the control group (FIG. 1). The 5′UTR and 3′UTR modifications are broadly applicable to mRNAs encoding cytokines and immune checkpoints regulators such as ICOS, 4-1BB, GITR, CD40, etc. Nanoparticles were formulated with lipids, DOPE, cholesterol, DMG-PEG, and mRNA (See, Li, B et al. An Orthogonal array optimization of lipid-like nanoparticles for mRNA delivery in vivo. Nano Lett. 2015, 15, 8099-8107).

### Example 2. Combination Therapy of NPs/OX40 mRNA+Anti-OX40 Antibody Improved Tumor Therapy in B16 Melanoma Tumor Model

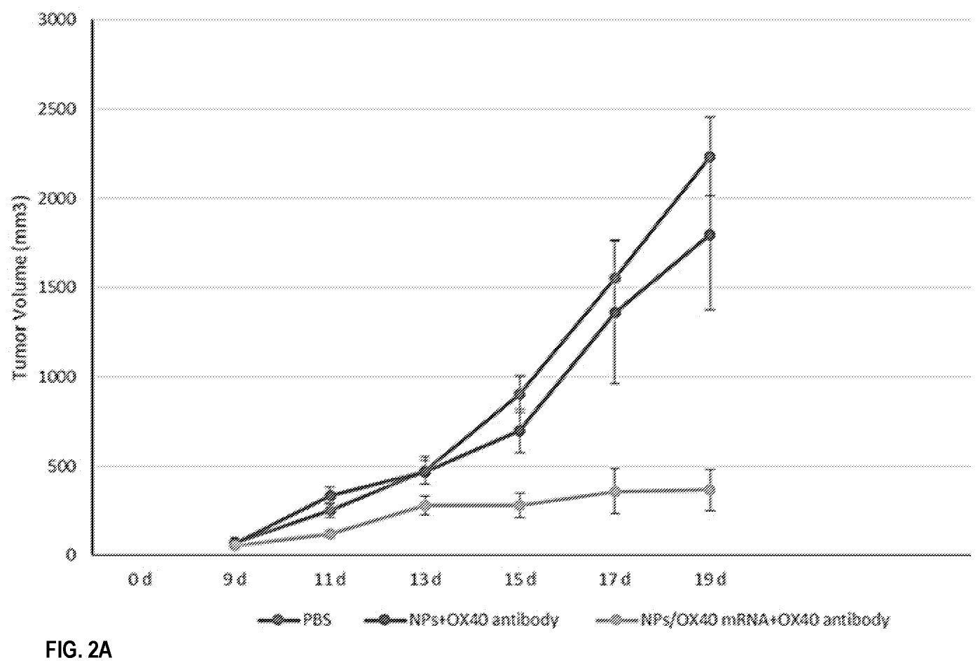

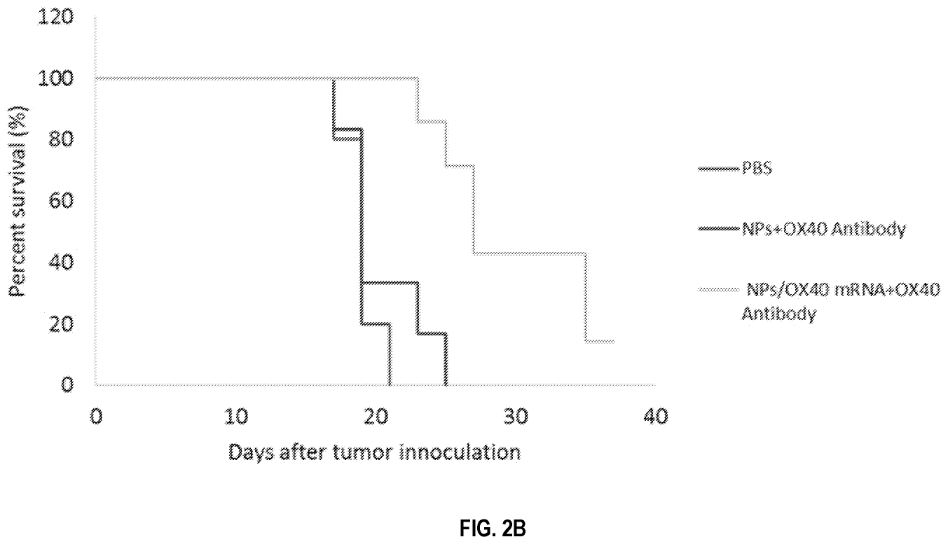

A B16 melanoma mouse tumor model was established (Triplett, T A, et al. Reversal of IDO-mediated cancer immune suppression by systemic kynurenine depletion with a therapeutic enzyme, Nat Biotechnol. 2018 September; 36(8): 758-764). Mice were treated with PBS, NPs+anti-OX40 antibody (InVivoPlus anti-mouse OX40 (clone OX-86) (Company: BioXcell, Catalog: BP0031)), or NPs/OX40 mRNA+anti-OX40 antibody. Combination of these mRNAs and their relevant antibodies significantly improved tumor therapy (FIG. 2A) and extended overall survival (FIG. 2B) in this mouse tumor model.

### Example 3. Combination Therapy of NPs/OX40 mRNA+Anti-OX40 Antibody Improved Tumor Therapy in CT26 Tumor Model

A CT26 mouse tumor model was established (Malvicini, M, et al. Tumor Microenvironment Remodeling by 4-Methylumbelliferone Boosts the Antitumor Effect of Combined Immunotherapy in Murine Colorectal Carcinoma, Molecular Therapy. vol. 23 no. 9, 1444-1455 September 2015). Mice were treated with PBS, nanoparticles (NPs)+anti-OX40 antibody, nanoparticles (NPs)/OX40 mRNA+anti-OX40 antibody and nanoparticles (NPs)/OX40 mRNA+anti-OX40 antibody together. nanoparticles (NPs)/OX40 mRNA+anti-OX40 antibody (injection interval: 6 h); and nanoparticles (NPs)/OX40 mRNA+anti-OX40 antibody together (injection interval: 0 h). OX40 antibody used was the InVivoPlus anti-mouse OX40 (clone OX-86) (Company: BioXcell, Catalog: BP0031)). Combination of these mRNAs and their relevant antibodies significantly extended overall survival and improved tumor therapy in a mouse tumor model.

### Example 4. Nanoparticles Comprising the mRNAs that Encodes the Co-Stimulatory Molecules

Cancer immunotherapy employs a variety of approaches to stimulate antitumor immune responses, including cancer vaccines, cell-based therapies, immune checkpoint blockers, monoclonal antibodies, mRNA-based immunotherapies, and other nanoparticle mediated immunotherapies. In particular, the use of immune checkpoint inhibitors has led to improved overall survival for cancer patients by targeting the T cell coinhibitory pathways such as PD-1 and CTLA-4. Although these antibodies are used routinely in the clinic, the percentage of patients that experience meaningful tumor responses is only about 25%. Therefore, there is an urgent need to develop new immunotherapy strategies for cancer treatment.

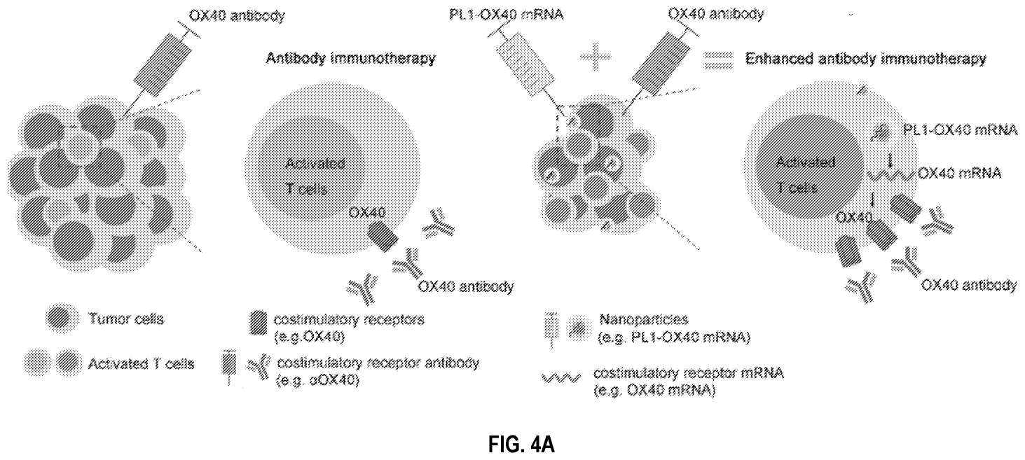

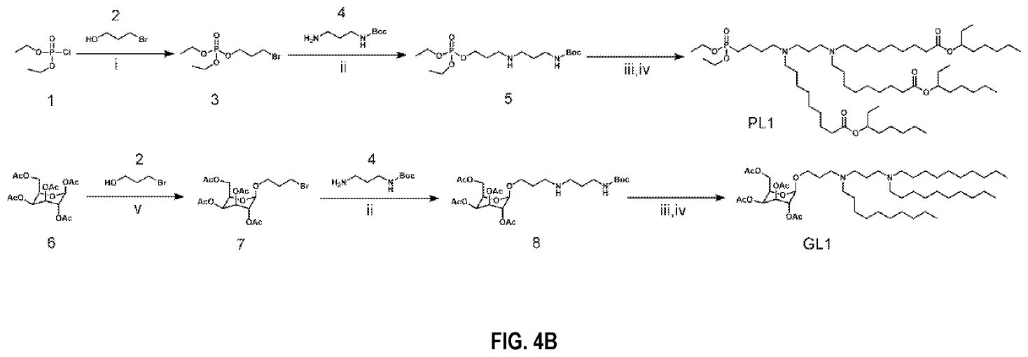

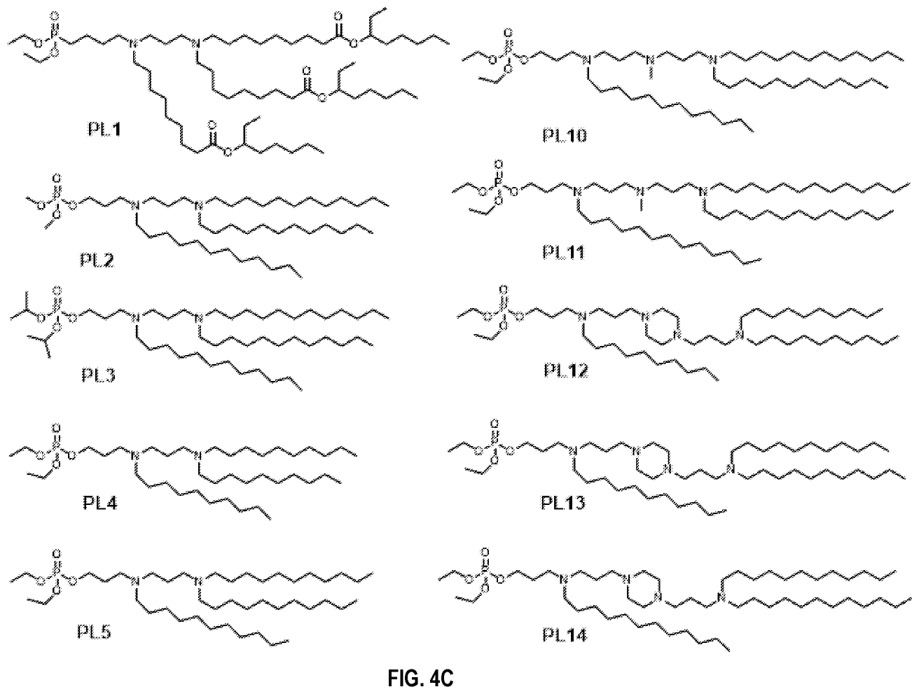

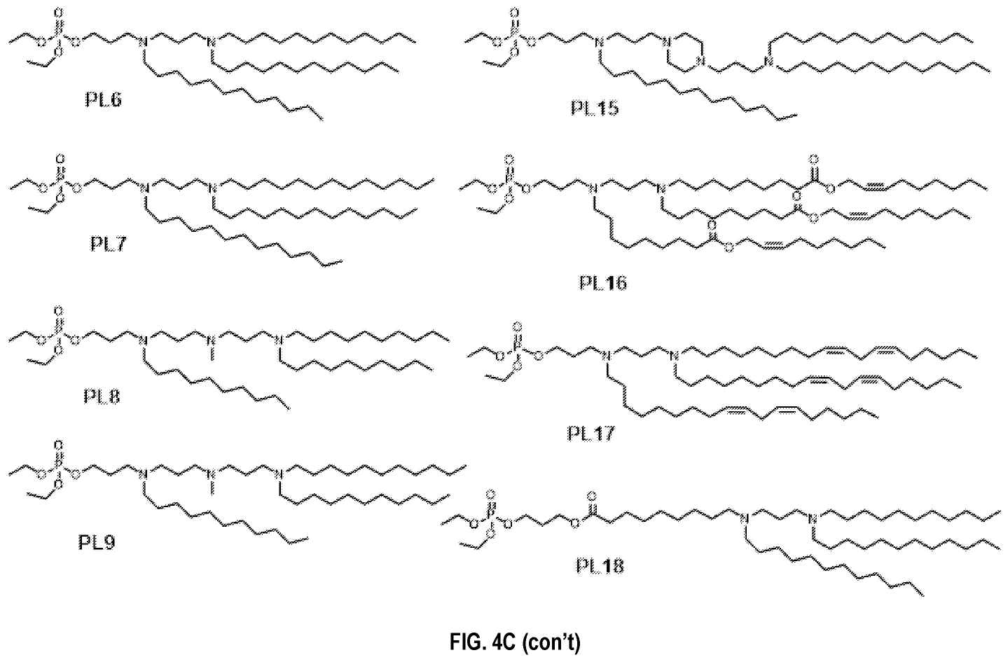

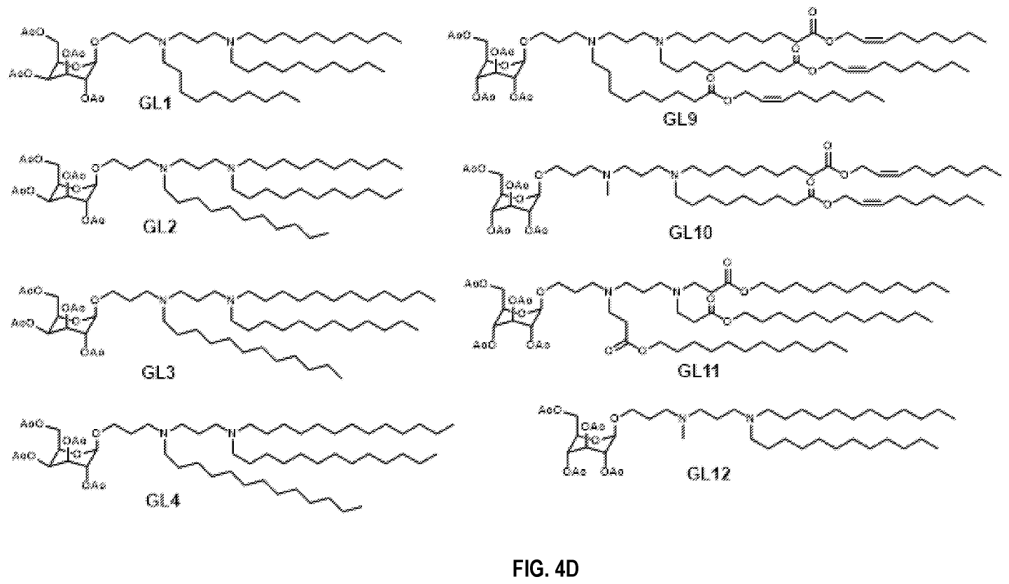

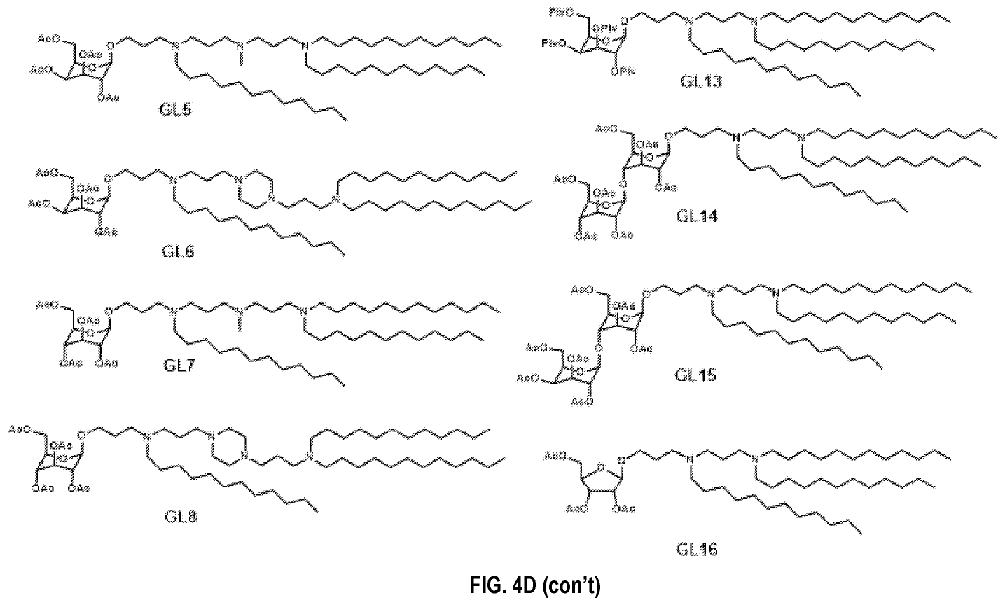

Recently, researchers discovered a series of costimulatory molecules on T cells for cancer immunotherapy. The interactions of the ligands of costimulatory molecules with their costimulatory receptors on the surface of T cells activate clonal T cell expansion and differentiation, thus leading to increased antitumor efficiency in several human cancers. CD137 (also known as 4-1BB) and OX40 (also known as CD134) are T cell costimulatory receptors and provide activating signals for CD8 and CD4 T cells. CD137 plays an important role in T cell proliferation and cytokine secretion. Recently, two anti-CD137 antibodies (urelumab and utomilumab) have been investigated in clinical trials. OX40 is involved in stimulating CD8+ T cells for the generation of anti-tumor immune responses. Anti-OX40 antibodies augment T cell differentiation, cytolytic function, and antitumor immunity in various cancer types. Several agonistic anti-OX40 antibodies are currently in clinical trials. Although costimulatory signals are critical to stimulate T cells, they express inadequately in tumor microenvironment, which impedes immunotherapeutic effects. Therefore, the delivery of costimulatory receptor mRNA into tumor-infiltrating T cells in combination with the use of agonistic antibody to that receptor can directly activate T cells and improve cancer immunotherapy (FIG. 4A).

To deliver costimulatory receptor mRNA into T cells, phospholipids and glycolipids were used because they are natural components of the cell membrane. Based on the chemical structures of phospholipids and glycolipids, a library of phospholipid and glycolipid mimetic materials were designed and synthesized (FIGS. 4B-4D). These compounds were formulated into phospholipid- and glycolipid-derived nanoparticles for mRNA delivery. One phospholipid-derived nanoparticle, PL1, efficiently delivered mRNA to T cells both in vitro and in vivo. Next, PL1 nanoparticles were used to deliver the costimulatory receptor CD137 or OX40 mRNA to tumor-infiltrating T cells in combination with anti-CD137 or anti-OX40 antibody in multiple tumor models. Moreover, this treatment approach significantly improved the immunotherapeutic effect of anti-PD-1+anti-CTLA-4 antibodies. This example provides a new and urgently needed biomaterial to deliver costimulatory receptor mRNA in order to activate T cells and boost anti-tumor immunity.

### Example 5. Design and Synthesis of Phospholipid and Glycolipid Derivatives (PLs and GLs) for mRNA Delivery

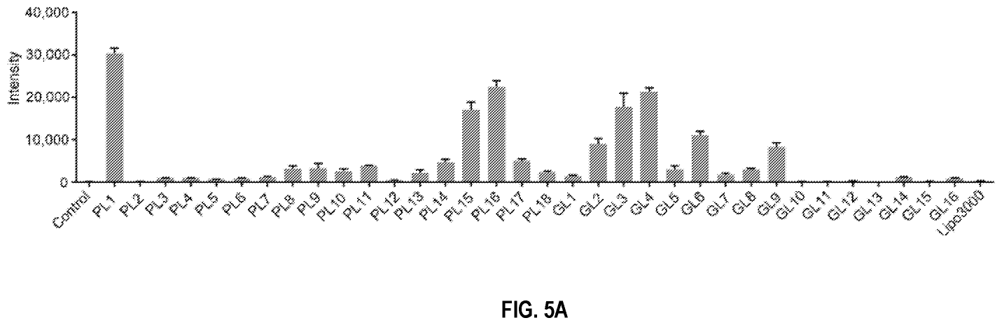

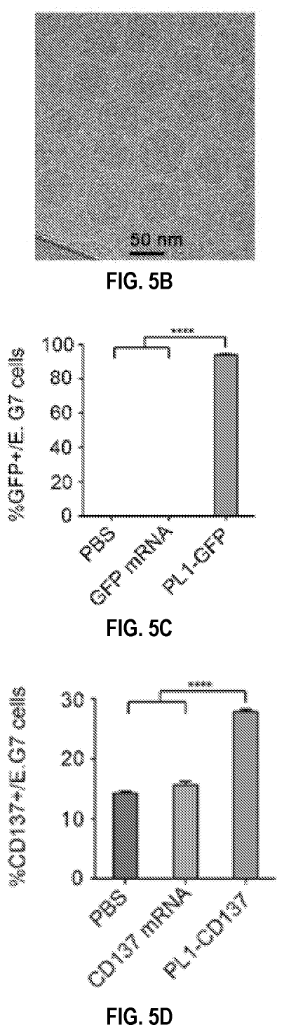

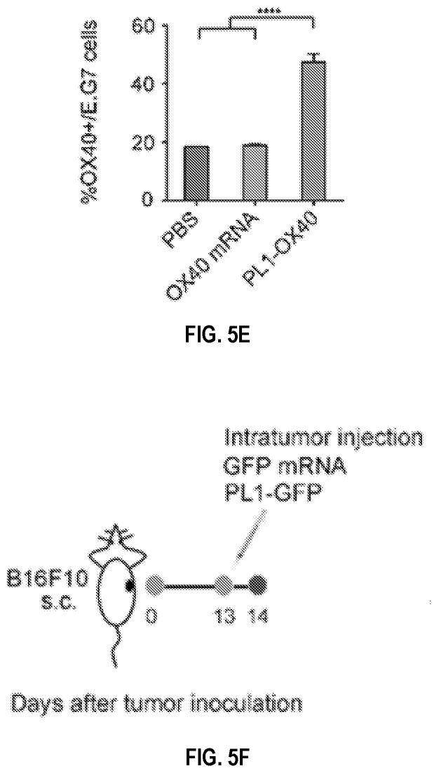

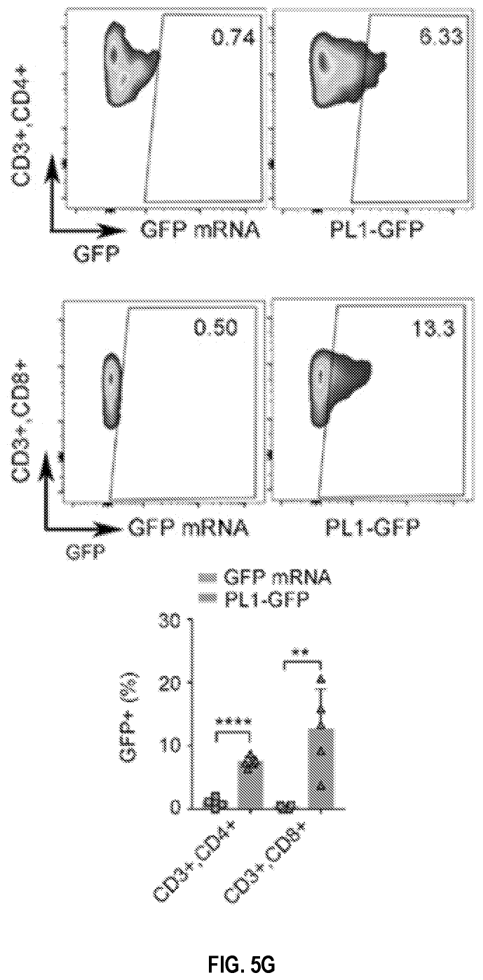

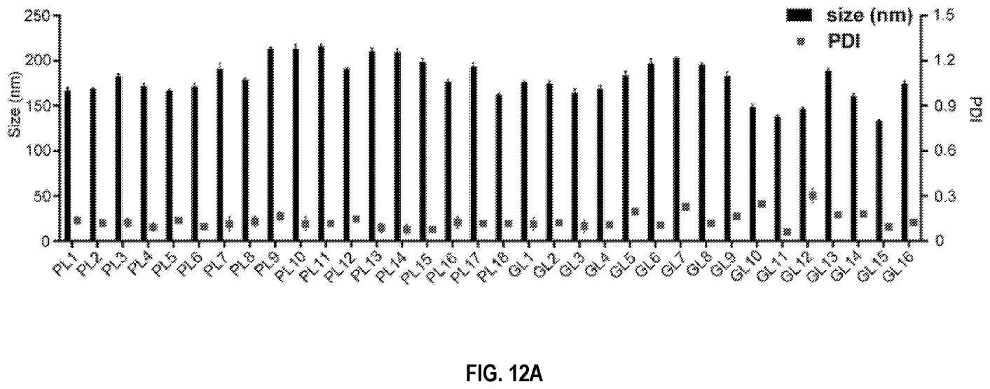

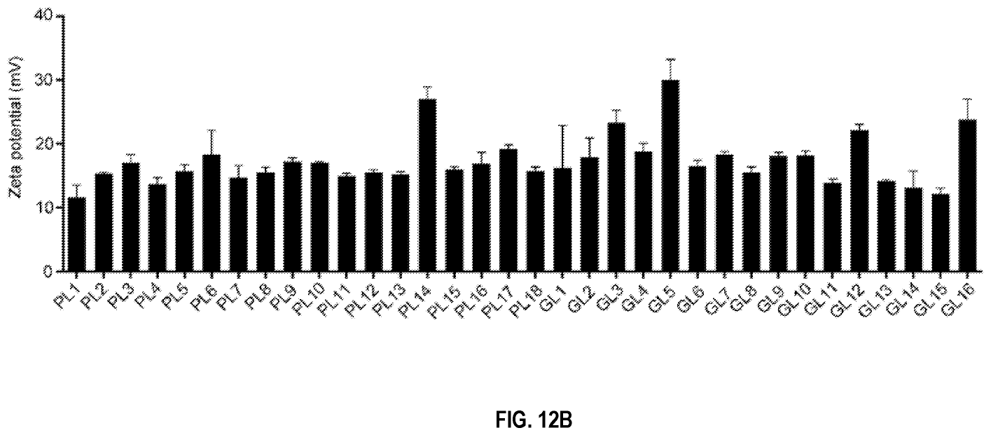

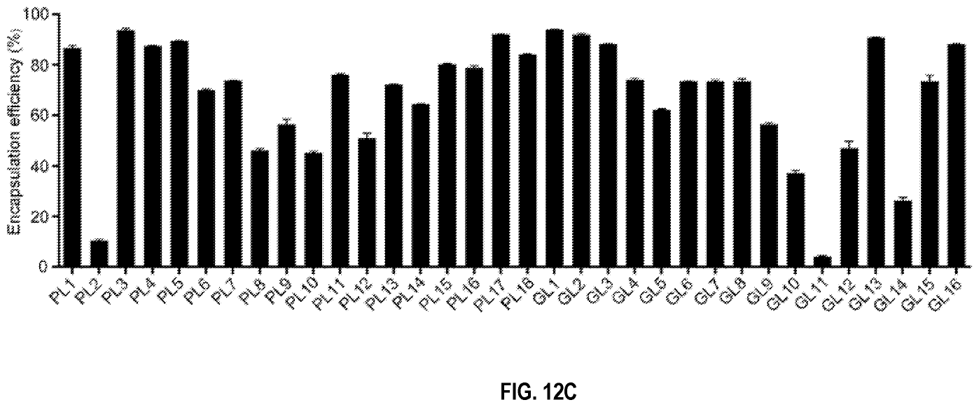

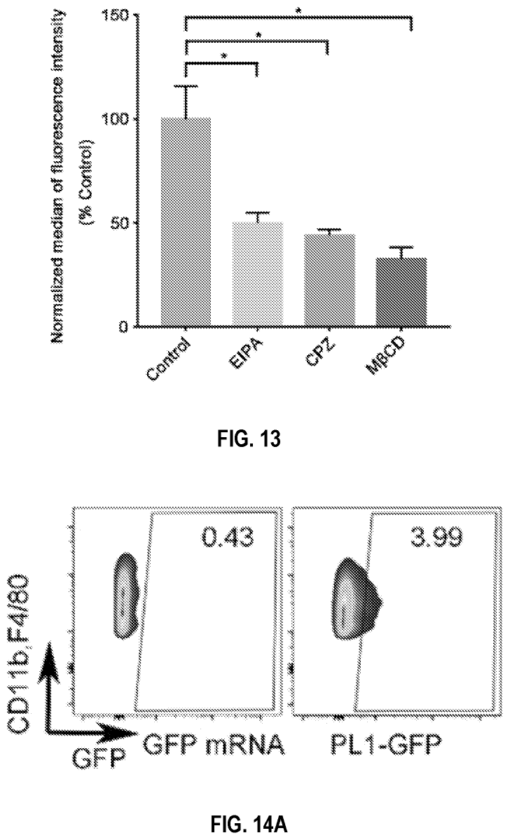

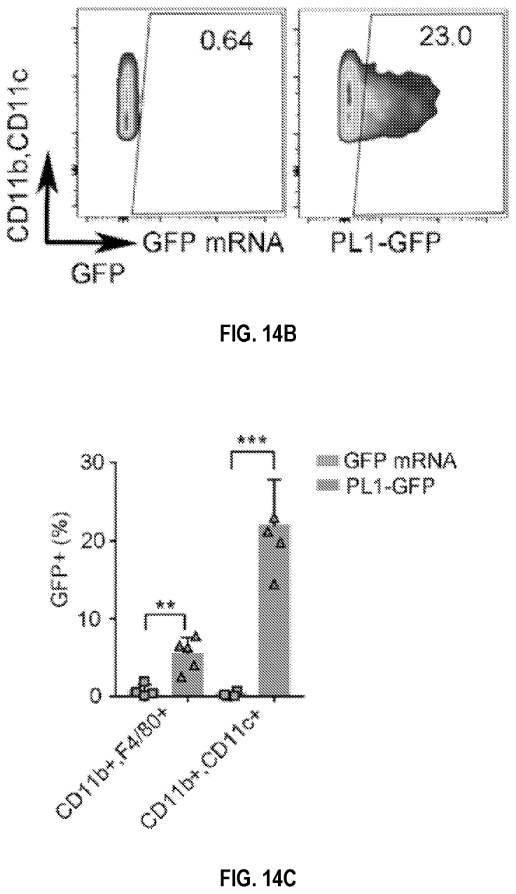

Biomimetic compounds, phospholipids and glycolipids, are composed of a biomimetic head (phosphate head or glyco head), an ionizable amino core, and multiple hydrophobic tails (FIG. 11). These phospholipid and glycolipid derivatives (PLs and GLs) were synthesized according to previously reported procedures. See, for example, WO/2019/027999. FIG. 4B shows representative synthetic routes to PL1 and GL1. Following this synthetic route, PL1-18 and GL1-16 materials were synthesized (FIG. 4C), which were characterized by 41 nuclear magnetic resonance (NMR) and mass spectroscopy (MS) (FIG. 4C). Next, PLs and GLs nanoparticles were formulated with firefly luciferase mRNA (FLuc mRNA), and were characterized according to size, surface charge, and mRNA encapsulation efficiency (FIGS. 12A-12C). Then, the mRNA delivery efficiency of PL1-18 and GL1-16 nanoparticles was studied in E.G7 cells (a T-lymphocyte cell line) and found PL1 nanoparticles displayed the highest delivery efficiency of FLuc mRNA (FIG. 5A). Moreover, PL1 delivered GFP mRNA to about 94% of E.G7 cells, demonstrating its function as a T-cell delivery vehicle (FIG. 5C). Endocytic pathways of the PL1 nanoparticles were further investigated using endocytic inhibitors including 5-(N-Methyl-N-isopropyl)amiloride (EIPA) for macropinocytosis, chlorpromazine hydrochlorides (CPZ) for clathrin-mediated endocytosis, and methyl-beta-syslodextrin (MβCD) for caveolae-mediated endocytosis. Treatment with EIPA, CPZ, and MβCD significantly inhibited 50%, 56%, and 39% cellular uptake of PL1 nanoparticles, respectively (FIG. 13), indicating that PL1 nanoparticles were internalized through multiple endocytic pathways. The T-cell costimulatory receptor CD137 mRNA and OX40 mRNA were also delivered into E.G7 cells. FIG. 5B shows the cryo-TEM image of PL1-OX40 nanoparticles. Flow cytometry results showed that both PL1-CD137 (27.8%) and PL1-OX40 (47.4%) significantly increased the cell surface expression of CD137 and OX40, respectively (FIGS. 5D and 5E). The next investigation was done on intratumoral (i.t.) delivery of PL1-GFP in tumor-infiltrating lymphocytes in a murine melanoma model (B16F10 melanoma cells growing s.c. in C57BL/6 mice) (FIG. 5F). With PL1-GFP treatment, increased expression of GFP was observed in tumor-infiltrating CD4+ and CD8+ T cells (FIG. 5G), as well as in macrophages and dendritic cells (DCs) (FIGS. 14A-14C). Based on these results, PL1 nanoparticles were chosen for delivering CD137 and OX40 mRNA in vivo.

### Example 6. Regression of Tumor Growth with the Treatment of PL1-CD137 mRNA+Anti-CD137 Antibody

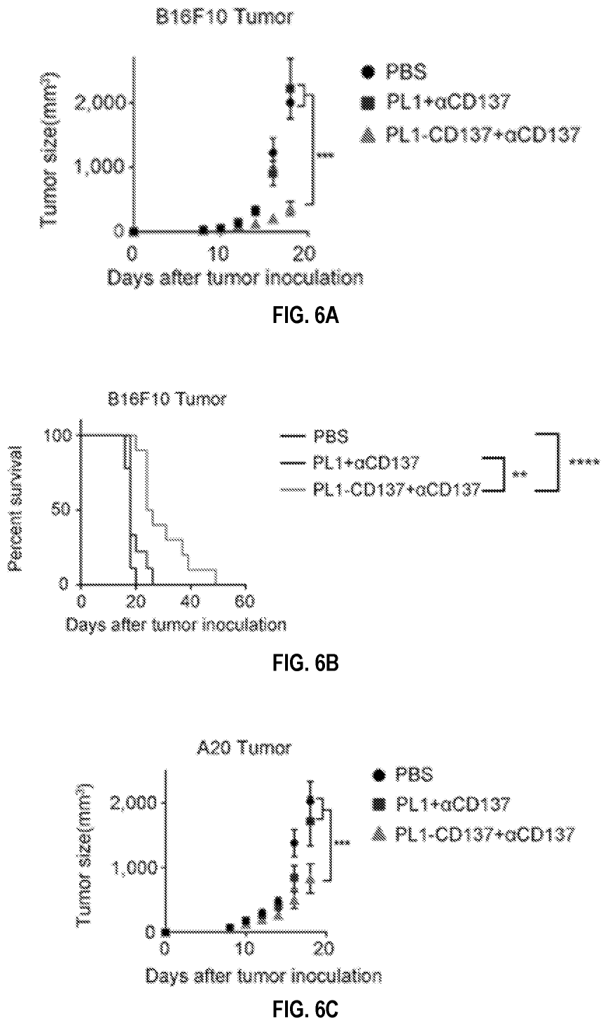

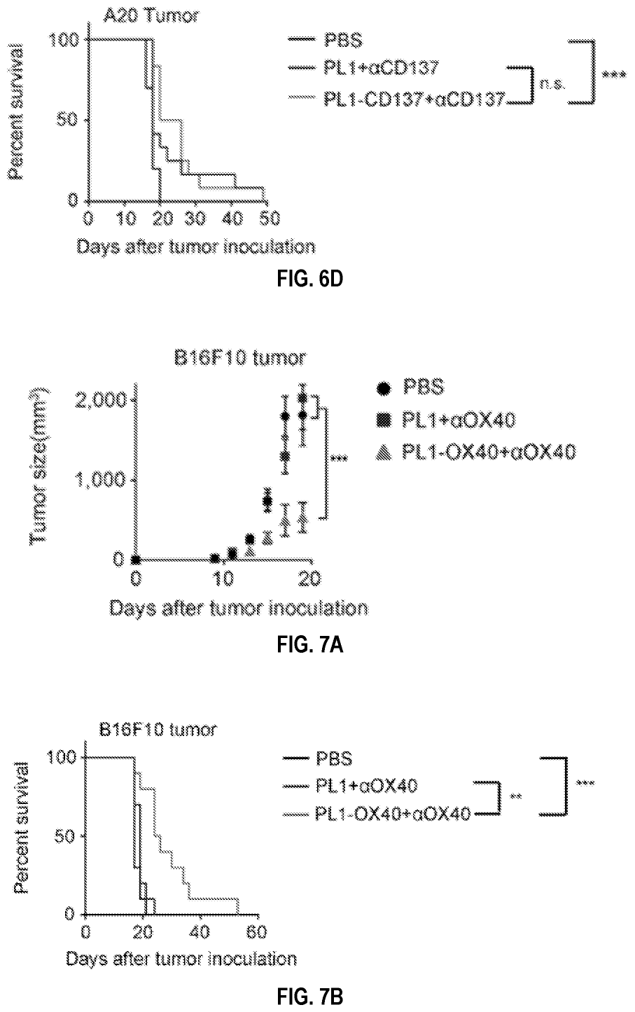

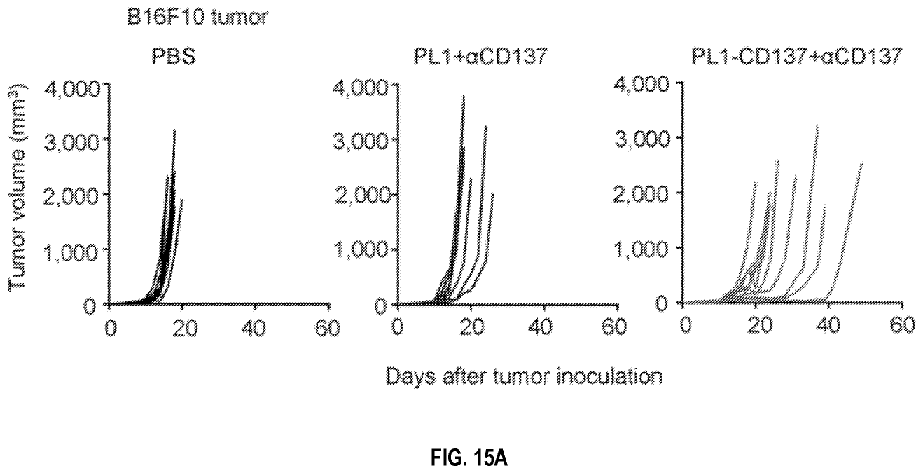

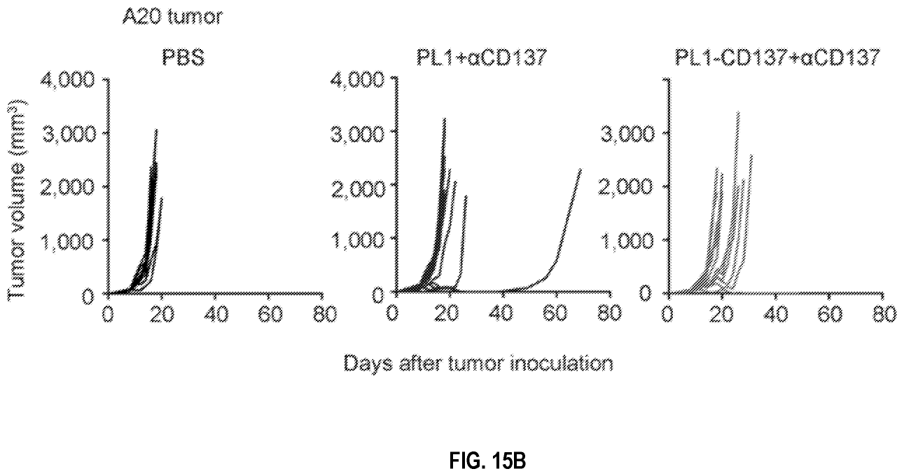

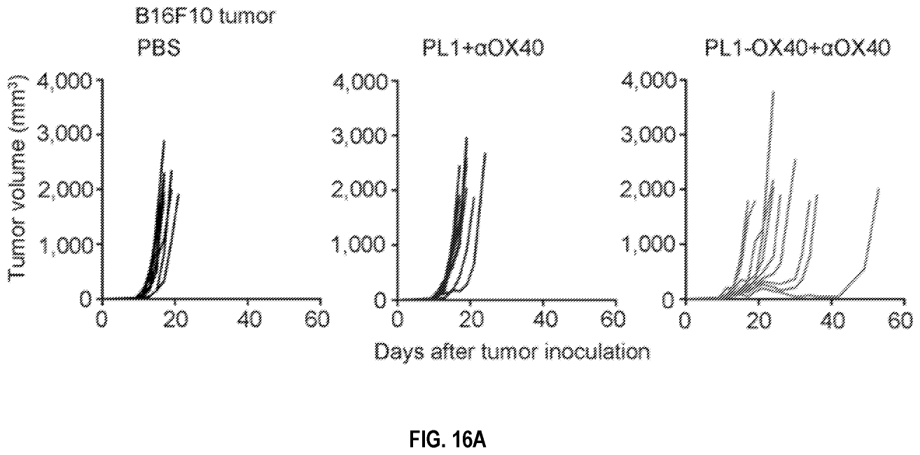

PL1-CD137 was intratumorally injected in combination with anti-CD137 antibody every other day for six doses in the B16F10 cell melanoma mouse model. Administration of PL1-CD137+anti-CD137 dramatically decreased the tumor growth rate (5-fold less than control, inoculation 18 d) (FIG. 6A and FIG. 15A). The treatment also significantly increased the overall survival time compared to PBS and PL1 (empty nanoparticle)+anti-CD137 Ab (FIG. 6B). Similar experiments were conducted in the A20 lymphoma tumor model. Treatment with PL1-CD137+anti-CD137 Ab resulted in a 2-fold decrease in the tumor growth rate (inoculation 18 d) in comparison to PBS and PL-1+anti-CD137 Ab (FIG. 6C and FIG. 15B). However, no significant extension in the overall survival time was observed comparing PL1-CD137+anti-CD137 Ab to PL-1+anti-CD137 Ab treatment (FIG. 6D). Thus, PL1 nanoparticle delivery of costimulatory receptor CD137 mRNA improved the results of immunotherapy with an anti-CD137 Ab to some extent in both tumor models with better results obtained in the B16F10 melanoma model as compared to the A20 lymphoma model.

### Example 7. Regression of Tumor Growth with the Treatment of PL1-OX40 mRNA+Anti-OX40 Antibody

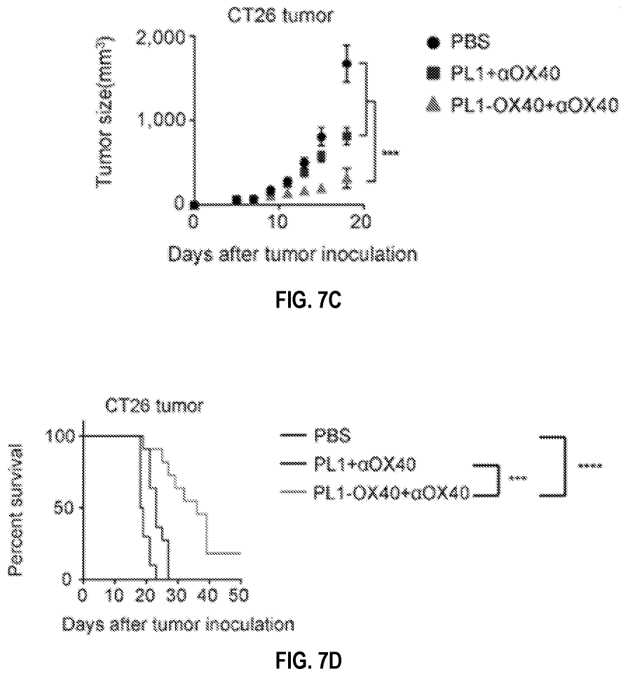

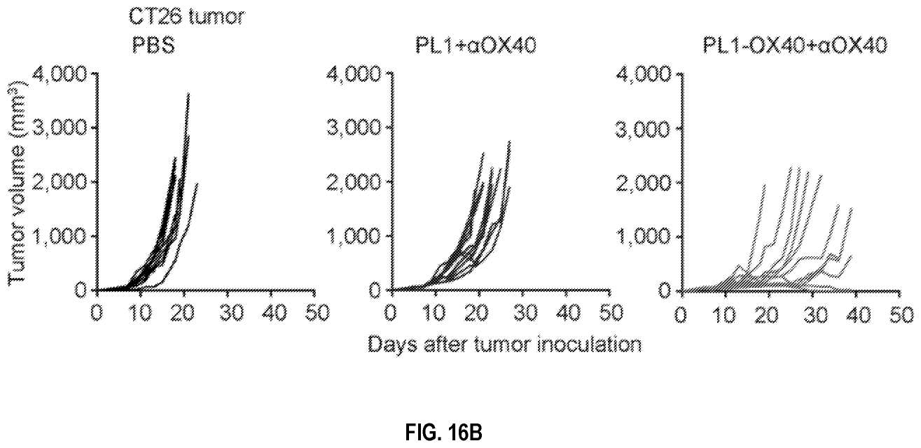

The therapeutic effects of the costimulatory receptor OX40 delivery in the B16F10 melanoma tumor model were also explored. PL1-OX40+anti-OX40 Ab treatment (i.t.) significantly decreased the tumor growth and prolonged the survival in comparison to treatment with PBS and PL1+anti-OX40 Ab (FIGS. 7A, 7B, and 16A). Next, a CT26 mouse tumor model was established in BABL/c mice. A significant therapeutic effect was observed following treatment with PL1-OX40+anti-OX40 Ab (FIGS. 7C, 7D, and 16B).

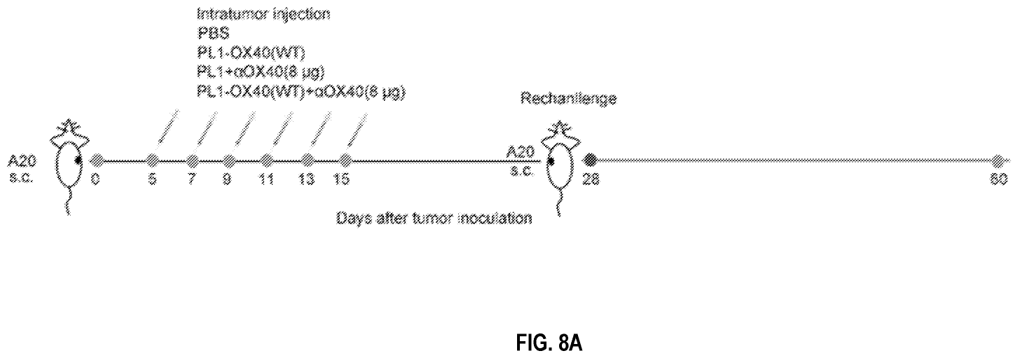

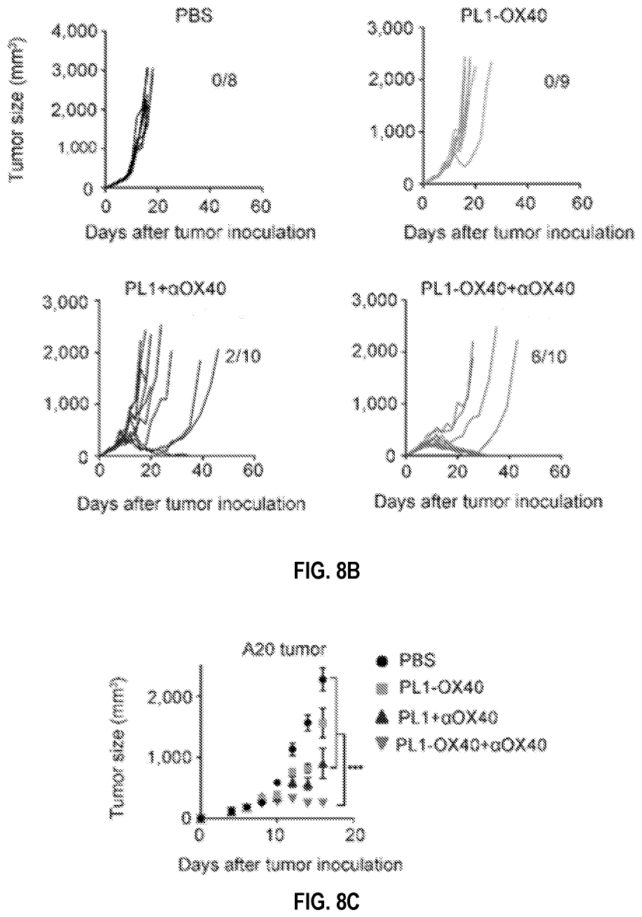

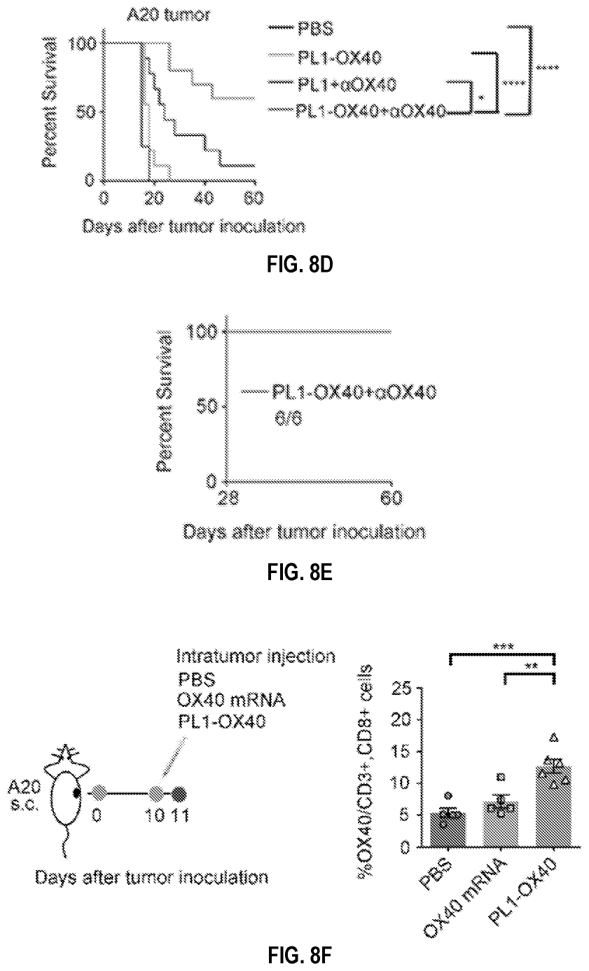

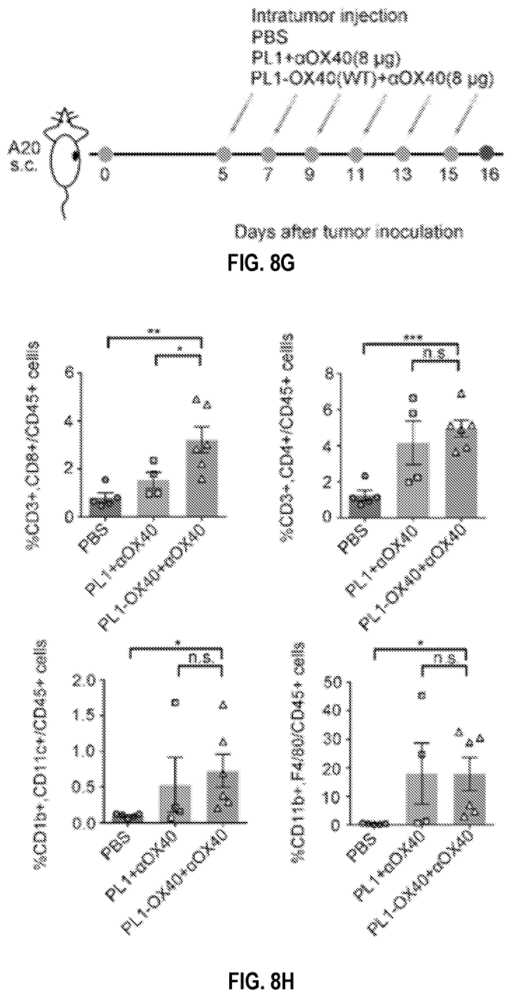

Next, the therapeutic effects of PL1-OX40+anti-OX40 Ab treatments were evaluated in the A20 B cell lymphoma model. Mice received i.t. injections of PBS, PL1-OX40, PL1+anti-OX40 Ab or PL1-OX40+anti-OX40 Ab. Tumor growth was monitored for 60 days (FIGS. 8A and 8B). Treatment with PL1-OX40+anti-OX40 Ab significantly reduced tumor growth (FIG. 8C) and increased the length of survival (FIG. 8D) compared with controls. Importantly, 6 out of 10 (60%) mice treated with PL1-OX40+anti-OX40 Ab exhibited a complete response (FIG. 8D) and were resistant to rechallenge with A20 tumor cells (FIG. 8E). These results indicated that PL1 nanoparticles delivering the co-stimulatory OX40 mRNA could enhance the immunotherapeutic effects of anti-OX40 Ab therapy in three different mouse models.

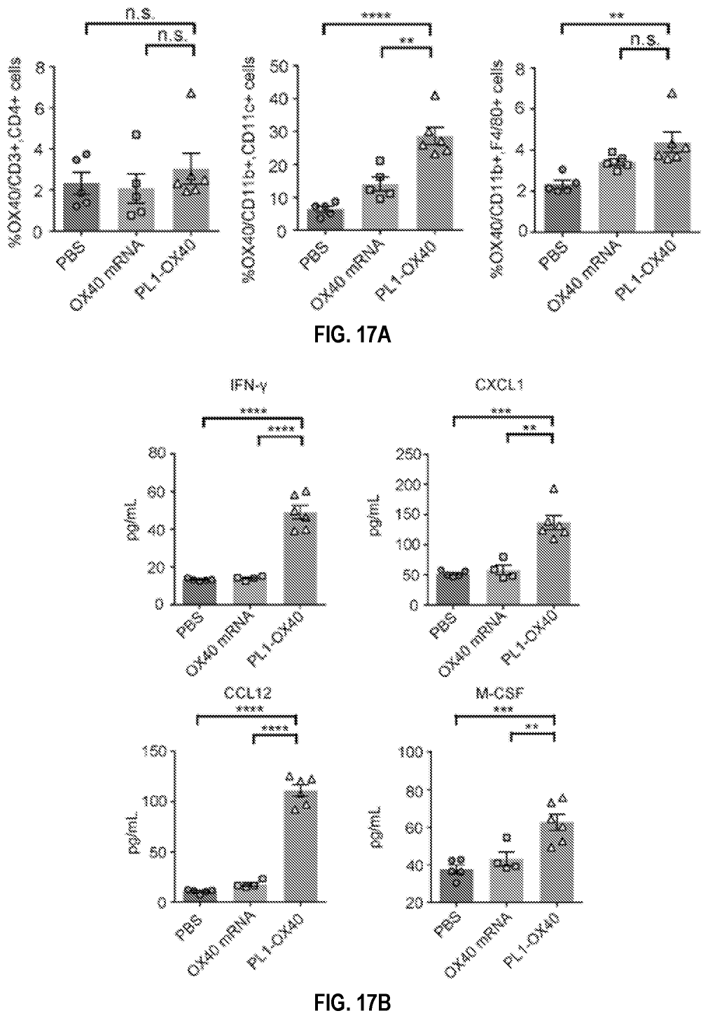

Tumor-infiltrating lymphocytes (TIL) play an essential role in anti-tumor immunity. mRNA Delivery to intratumoral T cells was explored in the A20 B cell lymphoma model. There was a significant increase in the expression of OX40 on tumor-infiltrating CD8+ T cells following PL1-OX40 treatment (FIG. 8F), but there were minimal changes in the OX40 expression on infiltrating CD4+ T cells or macrophages (FIG. 17A). There was also a significant increase in expression of OX40 on infiltrating dendritic cells (DCs) (FIG. 17A). Cytokine and chemokine levels were also examined. Plasma levels of IFN-γ were significantly increased following PL1-OX40 treatment compared to control groups, Levels of the chemokine ligand 12 (CCL12), neutrophil chemoattractant (CXCL1), and macrophage colony-stimulating factor (M-CSF) were also increased by 2 to 10-fold with the PL1-OX40 treatments (FIG. 17B).

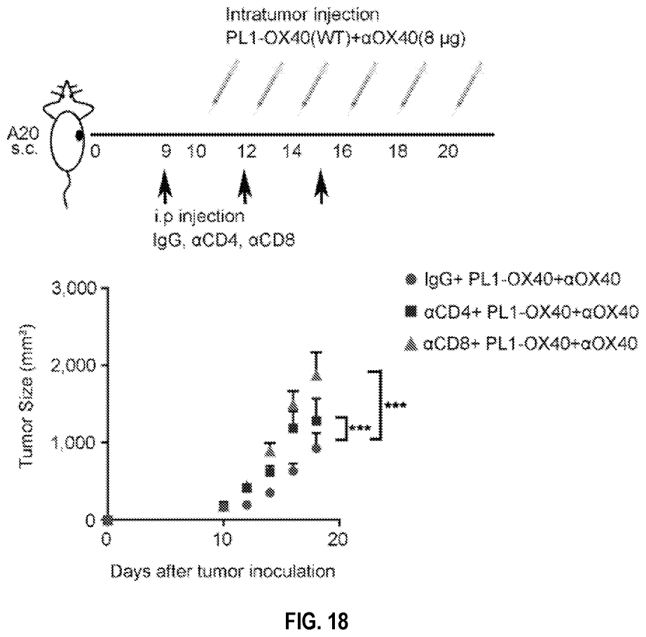

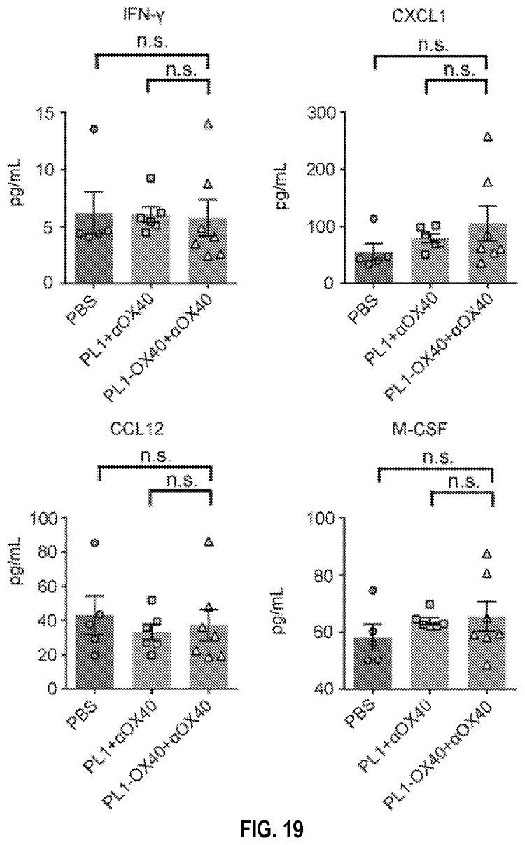

Infiltrating T-cell populations in A20 B cell lymphoma tumors were also examined. Using the same dosing strategy as described in FIG. 6A, immune cell populations were analyzed 24 hrs following the last treatment (FIG. 8G). A significant increase in CD8+ T cells was observed, but there was no change in levels of CD4+ T cells, macrophages, or dendritic cells with the PL1-OX40+anti-OX40 Ab treatment compared to the PL1+anti-OX40 Ab (FIG. 8H). Interestingly, T-cell depletion with either anti-CD8 or anti-CD4 Abs significantly compromised the efficacy of the combination treatment as compared to the administration of a control Ab (FIG. 18). The cytokine levels of IFN-γ, CCL12, M-CSF and CXCL1 in mouse plasma were similar in the different groups after six doses of treatment (FIG. 19).

### Example 8. Boosting Antitumor Efficacy of PL1-OX40 mRNA+Anti-OX40 Antibody

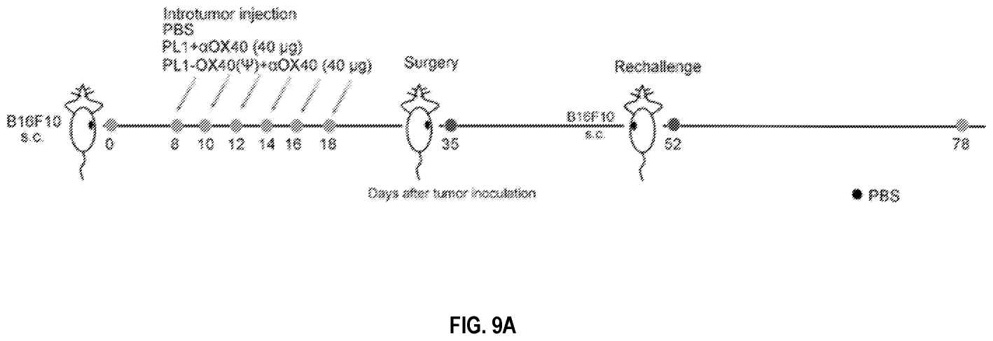

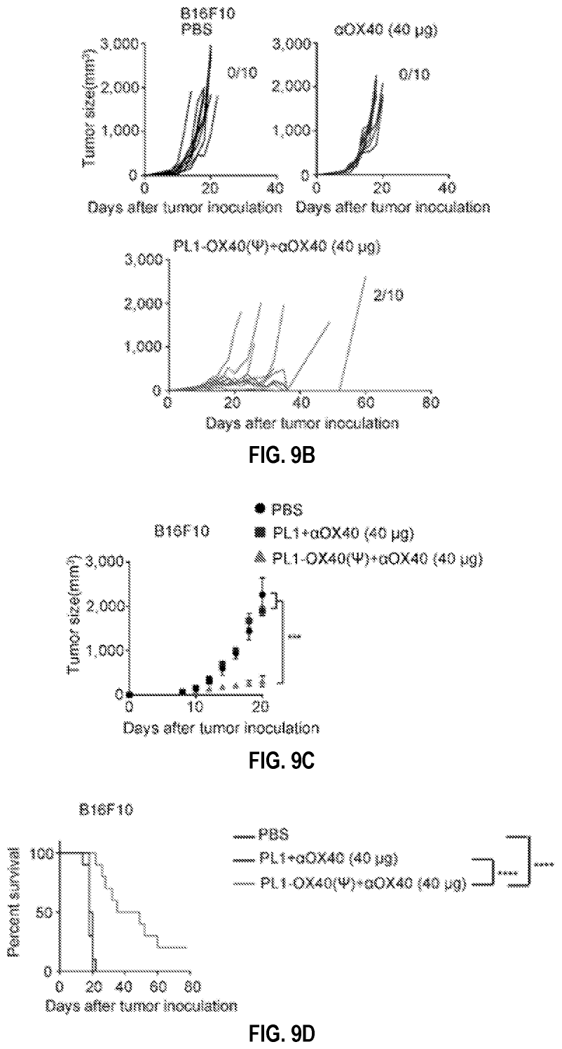

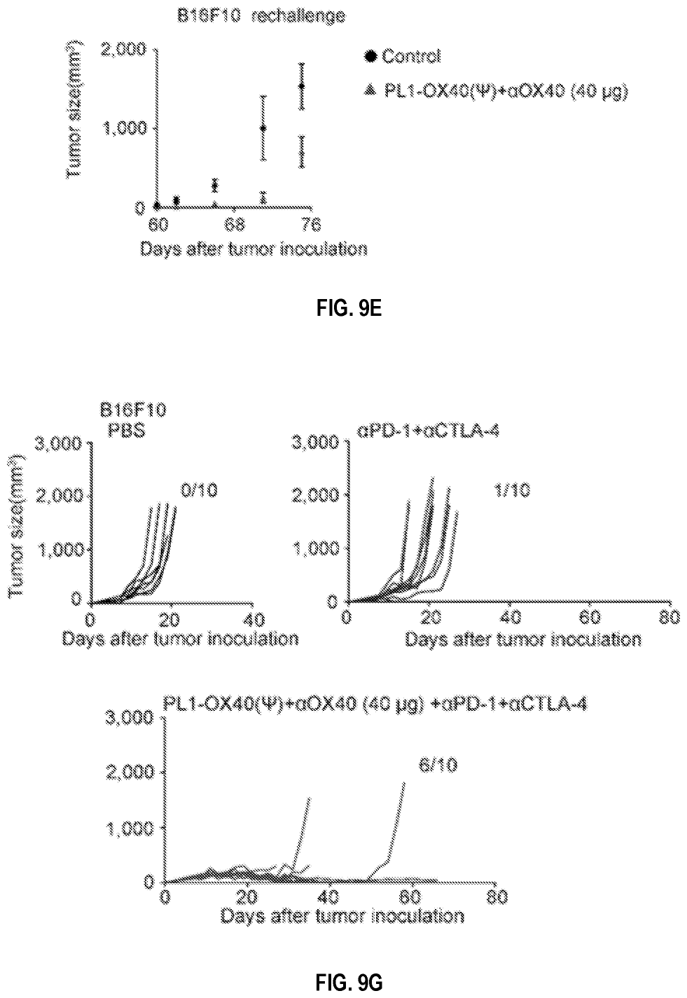

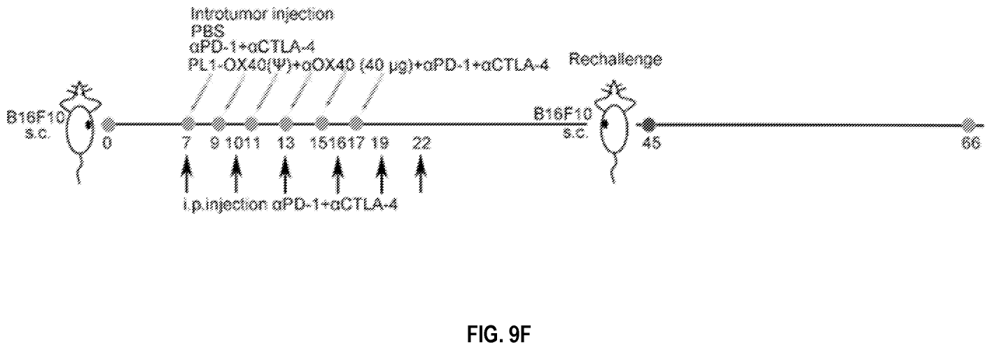

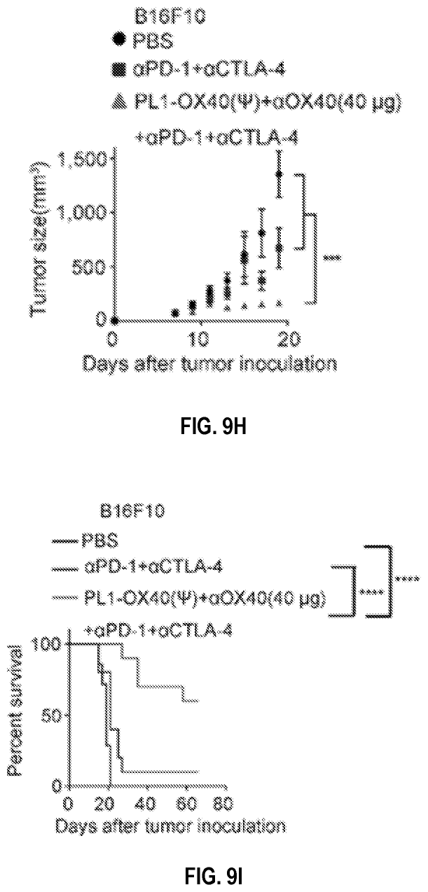

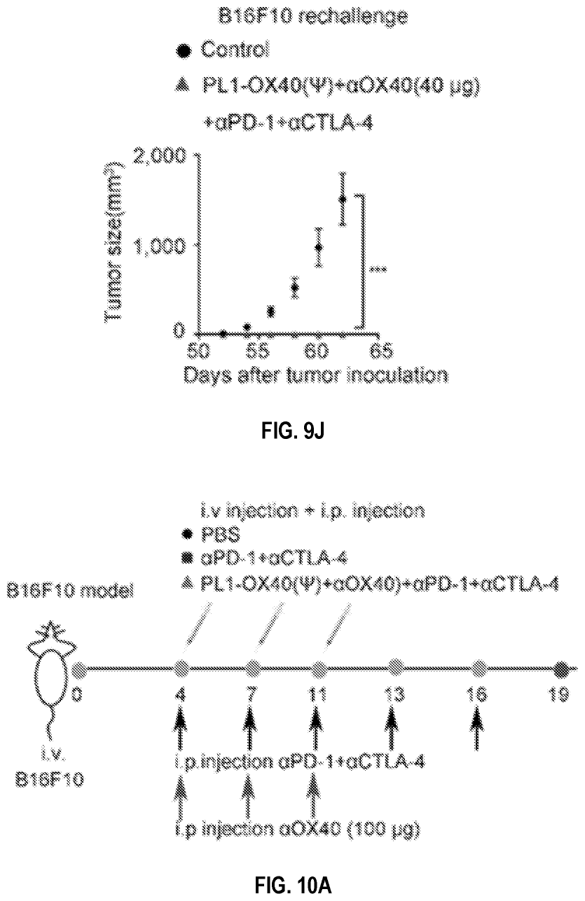

Although the PL1-OX40+anti-OX40 Ab treatments significantly decreased B16F10 tumor growth and prolonged survival (FIGS. 7A and 7B), complete eradication of the tumor burden is an important goal of immune-based treatments. To improve the anti-tumor effects of PL1-OX40+anti-OX40 Ab therapy, OX40 mRNA was modified from its wild-type form (OX40 (WT) to a pseudouridine (ψ)-modification (OX40 (ψ)), and anti-OX40 antibody doses were increased from 8 to 40 μg. Treatment of the B16F10 tumor bearing mice with PL1-OX40 (ψ)+anti-OX40 Ab (40 μg) significantly decreased tumor growth and prolonged survival in comparison to PBS and PL1+anti-OX40 treatment (FIGS. 9A-9D). At 35 d, five mice exhibited tumors with a size <500 mm3 and surgery was performed in order to remove the tumors from these mice. Two mice remained tumor free for over 50 days, which showed delayed tumor growth than the control mice with rechallenged B16F10 tumor cells (FIG. 9E).

In another treatment regimen, therapy with anti-PD-1+anti-CTLA-4 immune checkpoint inhibitor Abs were added to treatment with PL1-OX40 (ψ)+anti-OX40 Ab (40 μg) (FIG. 9F). The combination of PL1-OX40 (ψ)+anti-OX40 with anti-PD-1+anti-CTLA-4 Ab treatment dramatically inhibited tumor growth and prolonged survival in comparison to treatment with PBS or anti-PD-1+anti-CTLA-4 Ab (FIGS. 9G-9I). At 45 d, six mice were tumor free and one mouse had a small tumor (˜50 mm3). The surviving mice were resistant to the rechallenged B16F10 tumor cells (FIG. 9J), among which the primary tumor of one mouse re-grew and met early removal criteria on 58 d. The remaining 5 mice remained tumor free on both sides. These results indicate that the treatment regimen of PL1-OX40+anti-OX40 Ab improved the response to anti-PD-1+anti-CTLA-4 Ab therapy.

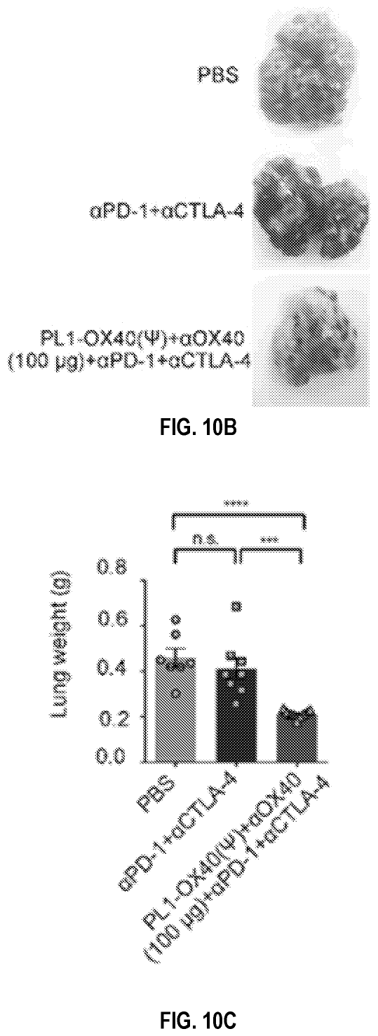

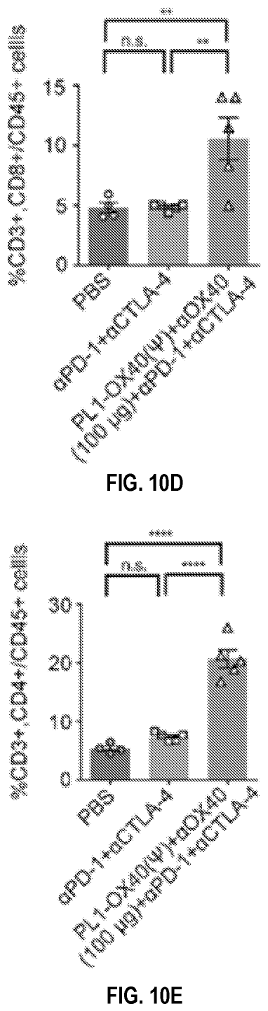

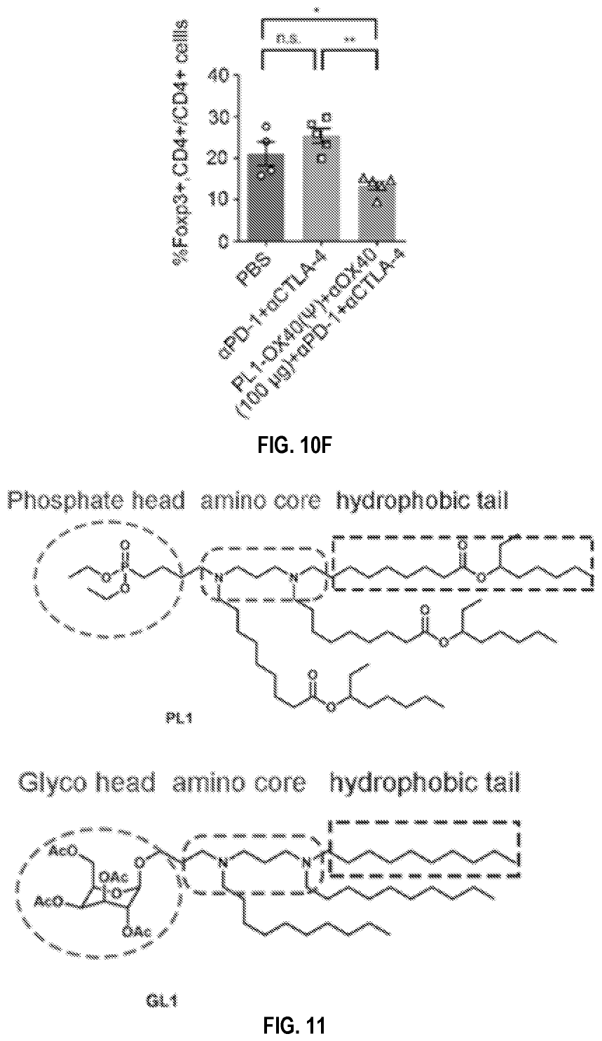

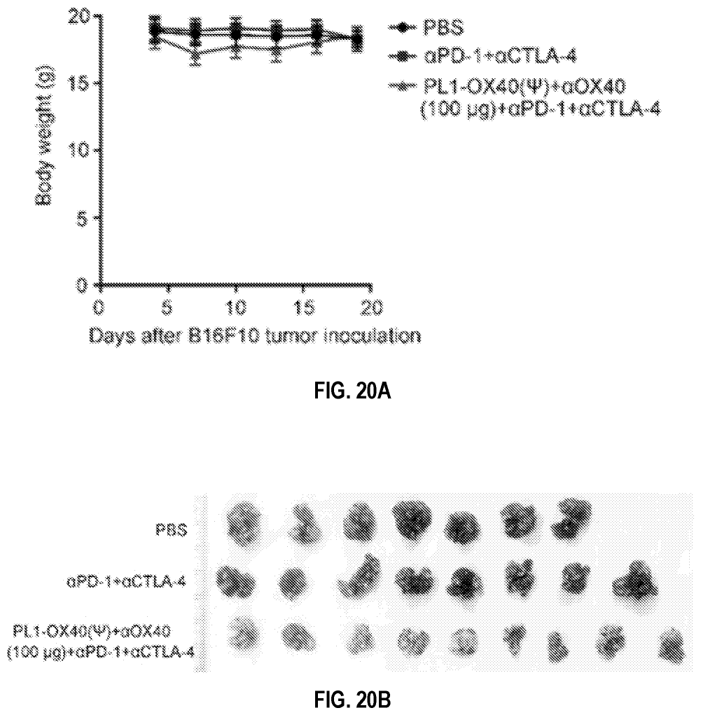

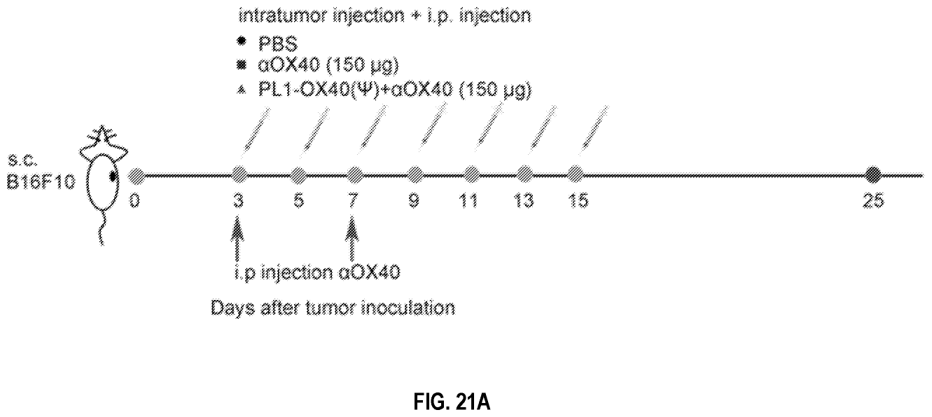

Then, the therapeutic efficacy of this treatment regimen was assessed using a B16F10 lung metastasis mouse model through systemic administrations of anti-PD-1+anti-CTLA-4 Abs with PL1-OX40 (ψ)+anti-OX40 Ab (100 μg) (FIG. 10A). The results showed that this treatment regimen dramatically reduced the tumor metastasis in mouse lungs compared with anti-PD-1+anti-CTLA-4 Abs and PBS treatment (FIGS. 10B-10C, FIGS. 20A-20B). A significant increase of CD8+ and CD4+ T cells in mouse lungs were observed in the group of PL1-OX40 (ψ)+anti-OX40 Ab with anti-PD-1+anti-CTLA-4 Abs compared to anti-PD-1+anti-CTLA-4 Abs treatment (FIGS. 10D-10E). Also, the number of Foxp3+CD4+ cells (Treg cells) was decreased in the lungs in the group of PL1-OX40 (ψ)+anti-OX40 Ab with anti-PD-1+anti-CTLA-4 Abs (FIG. 10F). These results indicate that systemic administrations of the treatment regimen demonstrate strong anti-tumor activity in the lung metastasis mouse model.

Agonist antibodies can be replaced by moieties with similar functions such as endogenous ligands. For examples, OX40 costimulatory receptor can interact with OX40 ligand. The coding sequence of OX40 ligand is shown in SEQ ID NO: 13.

### Example 9. Discussion

T cell-based immunotherapy of cancer is a rapidly developing field. Recently, nanotechnology has been developed to improve T cell therapy, such as ex vivo engineered T cells and in vivo modulation of T cells. Despite these significant advances, an important challenge remains: to stimulate anti-tumor immunity of primary T cells in vivo.

In this study, in order to explore nanoparticles for delivering mRNA into T cells, a library of phospholipid and glycolipid derivatives (PLs and GLs) were designed and synthesized. These materials were used to formulate biomimetic nanoparticles for mRNA delivery. PL1 nanoparticles not only delivered the costimulatory receptor mRNA to a T cell line in vitro, but was also able to deliver costimulatory receptor mRNA to T cells within the tumor, which provided a useful delivery tool for the modulation of T cell function.

Recently, agnostic antibodies (mAbs) specific for costimulatory receptors with the ability to boost anti-tumor T-cell immunity have been developed for cancer treatment. For example, the anti-OX40 antibody is able to activate T cells and enable them to remove tumor cells. However, low expression of OX40 hampered the anti-OX40 antibody immunotherapy effects in many tumor models (e.g. B16F10). In this study, PL1 nanoparticles were used to deliver OX40 mRNA into tumor-infiltrating T cells, which increased the expression of OX40 and consequently improved the antitumor effectiveness of anti-OX40 antibody. A combination treatment with PL1-OX40 and anti-OX40 antibody exhibited significant antitumor activity compared to the antibody alone in multiple tumor models. To further boost the anti-tumor activity of PL1-OX40 and anti-OX40 antibody, anti-PD-1+anti-CTLA-4 antibodies were added to the treatment regimen. This treatment approach resulted in an approximate 50% complete response in the B16F10 tumor model. Notably, these mice were resistant to a rechallenge with B16F10 tumor cells. This result indicates that this treatment regimen effectively induces anti-tumor immunity in vivo.

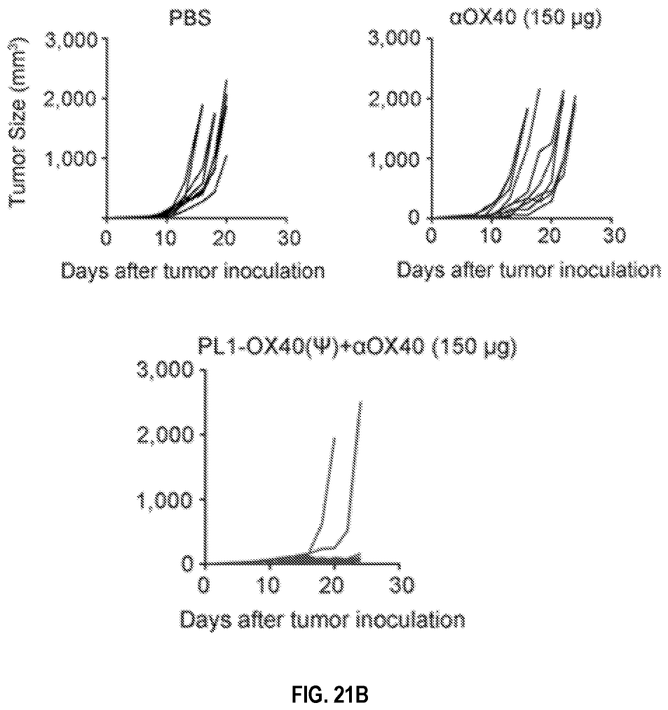

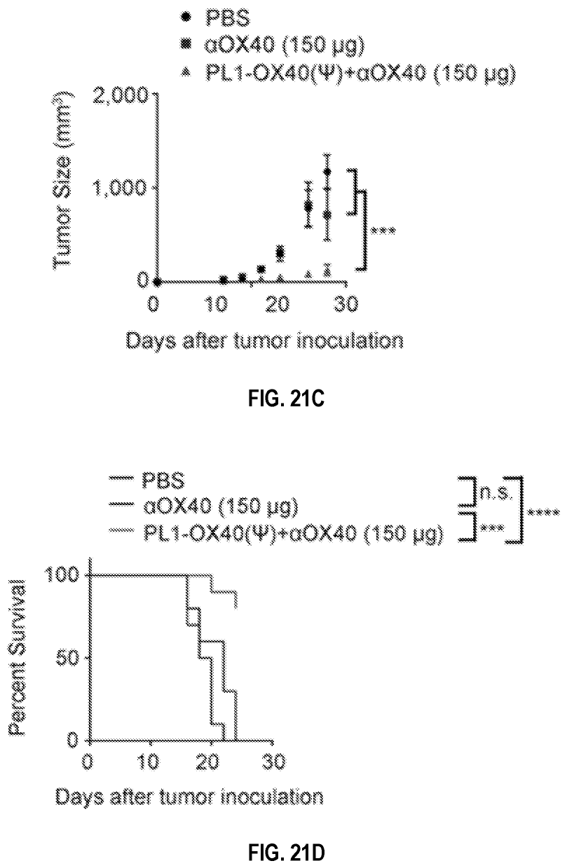

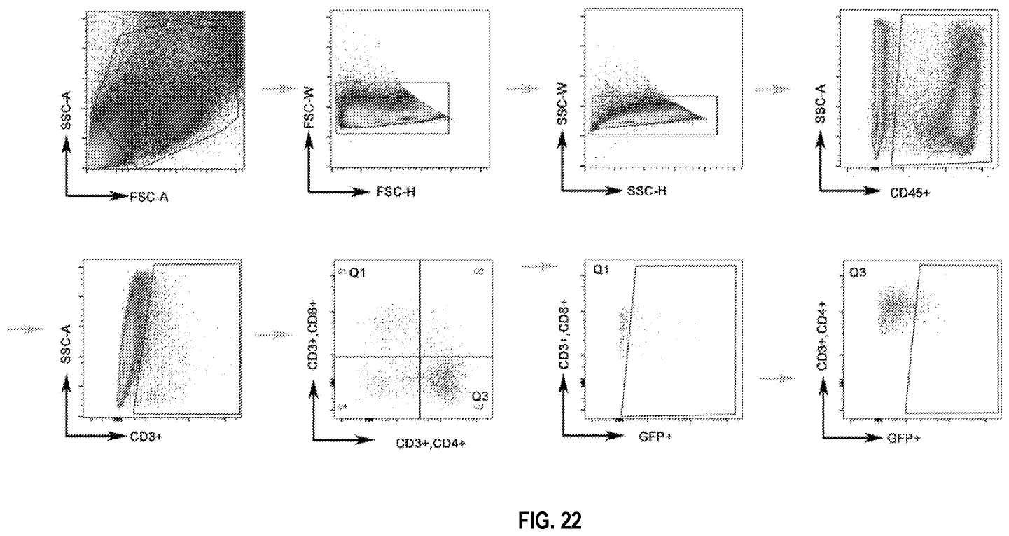

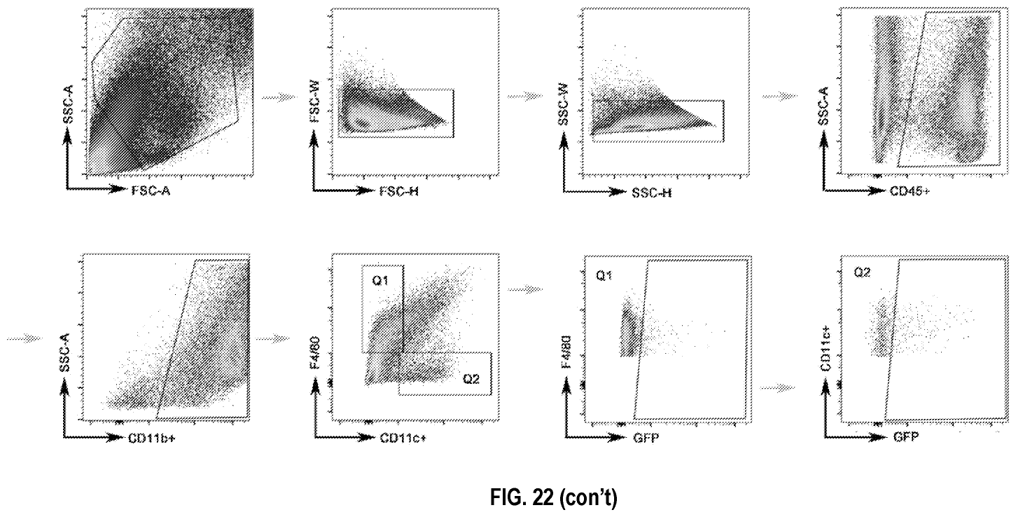

Furthermore, this treatment strategy is compatible to multiple administration routes. For example, the combination treatment with PL1-OX40 and anti-OX40 antibody exhibited significant antitumor activity through not only local administrations into the tumors, but also systemic administrations of anti-OX40 antibody (FIGS. 21A-21D). More importantly, systemic administrations of anti-PD-1+anti-CTLA-4 Abs with PL1-OX40 (ψ)+anti-OX40 Ab dramatically reduced the tumor metastasis in the lung metastasis model. These results demonstrate broad applicability of this treatment regimen under diverse therapeutic situations.

### Example 10. Chemical Synthesis of Phospholipid and Glycolipid Derivatives (PLs and GLs)

Phospholipid and glycolipid compounds and their analogues were synthesized according to the methods reported previously. See, for example, WO/2019/027999. General methods for PL1-PL18 and GL1-GL16 is to a solution of compound i or analogues (0.5 mmole) in CH₂Cl₂ (2 mL) was added excess amount of trifluoroacetic acid (1 mL). The mixture was stirred at RT for 2 h and monitored with thin layer chromatography. Upon completion of the reaction, the solvent was evaporated to yield an oil like intermediate. The intermediate was dissolved in 10 mL of anhydrous tetrahydrofuran, followed by adding triethylamine (0.2 mL). The resulting mixture was stirred for 30 min at RT. After adding aldehyde (3 mmole) and NaBH(OAc)₃ (3 mmole), the reaction mixture was stirred at RT for 24 h. After the solvent was removed, the reacting mixture was purified by column chromatography using a CombiFlash Rf system with a RediSep Gold Resolution silica column (Teledyne Isco) with the gradient elution (CH₂Cl₂ and ultra) from 100% CH₂Cl₂ to 70% CH₂Cl₂ (ultra, CH₂Cl₂/MeOH/NH₄OH=75/22/3 by volume) to give the corresponding products.

PL1, yield 34%. ¹H NMR (400 MHz, CDCl₃) δ=4.86-4.80 (3H, m), 4.16-4.08 (6H, m), 2.53-2.50 (2H, t, J=8), 2.42-2.37 (8H, m), 2.31-2.28 (6H, t, J=8), 1.83-1.80 (2H, m), 1.63-1.51 (21H, m), 1.37-1.28 (54H, m), 0.90-0.87 (18H, t, J=8). MS (m/z): [M+H]⁺ calcd. for C₆₁H₁₂₂N₂O₁₀P, 1073.88, found, 1073.88.

PL2, yield 64%. ¹H NMR (400 MHz, CDCl₃) δ=4.13-4.08 (2H, m), 3.79 (3H, s), 3.76 (3H, s), 2.53-2.50 (2H, t, J=8), 2.42-2.37 (9H, m), 1.85-1.78 (2H, m), 1.62-1.57 (2H, m), 1.45-1.43 (6H, m), 1.27 (54H, s), 0.91-0.87 (9H, t, J=8). MS (m/z): [M+H]⁺ calcd. for C₄₄H₉₄N₂O₄P, 745.70, found, 745.69.

PL3, yield 50%. ¹H NMR (400 MHz, CDCl₃) δ=4.86-4.80 (3H, m), 4.08-4.03 (2H, m), 2.54-2.51 (2H, t, J=8), 2.46-2.38 (9H, m), 1.83-1.80 (2H, m), 1.62-1.60 (2H, m), 1.45 (8H, m), 1.35-1.34 (12H, m), 1.28 (49H, s), 0.91-0.88 (9H, t, J=8). MS (m/z): [M+H]⁺ calcd. for C₄₈H₁₀₂N₂O₄P, 801.76, found, 801.76.

PL4, yield 48%. ¹H NMR (400 MHz, CDCl₃) δ=4.16-4.07 (6H, m), 2.54-2.50 (2H, t, J=8), 2.43-2.37 (9H, m), 1.84-1.80 (2H, m), 1.59-1.56 (2H, m), 1.45 (6H, m), 1.38-1.28 (49H, m), 0.91-0.88 (9H, t, J=8). MS (m/z): [M+H]⁺ calcd. for C₄₀H₈₆N₂O₄P, 689.63, found, 689.63.

PL5, yield 40%. ¹H NMR (400 MHz, CDCl₃) δ=4.14-4.07 (6H, m), 2.54-2.50 (2H, t, J=8), 2.43-2.37 (8H, m), 1.84-1.80 (2H, m), 1.59-1.56 (2H, m), 1.45 (6H, m), 1.37-1.28 (55H, m), 0.91-0.88 (9H, t, J=8). MS (m/z): [M+H]⁺ calcd. for C₄₃H₉₂N₂O₄P, 731.68, found, 731.68.

PL6, yield 48%. ¹H NMR (400 MHz, CDCl₃) δ=4.16-4.07 (6H, m), 3.72-3.69 (2H, m), 2.54-2.50 (2H, t, J=8), 2.44-2.38 (8H, m), 1.86-1.79 (2H, m), 1.72-1.69 (2H, m), 1.44 (6H, m), 1.37-1.34 (6H, m), 1.27 (54H, s), 0.91-0.88 (9H, t, J=8). MS (m/z): [M+H]⁺ calcd. for C₄₆H₉₈N₂O₄P, 773.73, found, 773.73.

PL7, yield 41%. ¹H NMR (400 MHz, CDCl₃) δ=4.16-4.07 (6H, m), 3.72-3.69 (2H, m), 2.54-2.50 (2H, t, J=8), 2.44-2.38 (8H, m), 1.86-1.79 (2H, m), 1.72-1.69 (2H, m), 1.44 (6H, m), 1.37-1.34 (6H, m), 1.27 (54H, s), 0.91-0.88 (9H, t, J=8). MS (m/z): [M+H]⁺ calcd. for C₄₉H₁₀₄N₂O₄P, 815.77, found, 815.77.

PL8, yield 26%. ¹H NMR (400 MHz, CDCl₃) δ=4.14-4.08 (6H, m), 3.24-3.22 (2H, m), 2.80-2.77 (1H, t, J=8), 2.54-2.50 (2H, t, J=8), 2.46-2.32 (14H, m), 2.22 (3H, s), 1.83-1.80 (2H, m), 1.65-1.60 (6H, m), 1.44 (5H, m), 1.37-1.28 (50H, m), 0.91-0.88 (9H, t, J=8). MS (m/z): [M+H]⁺ calcd. for C₄₄H₉₅N₃O₄P, 760.71, found, 760.71.

PL9, yield 24%. ¹H NMR (400 MHz, CDCl₃) δ=4.16-4.07 (6H, m), 2.80-2.76 (2H, t, J=8), 2.74-2.70 (4H, m), 2.61-2.58 (2H, m), 2.53-2.44 (8H, m), 2.31 (3H, s), 1.87-1.81 (4H, m), 1.69-1.66 (2H, m), 1.56 (4H, m), 1.44 (2H, m), 1.37-1.27 (54H, m), 0.91-0.87 (9H, t, J=8). MS (m/z): [M+H]⁺ calcd. for C₄₇H₁₀₁N₃O₄P, 802.75, found, 802.75.

PL10, yield 41%. ¹H NMR (400 MHz, CDCl₃) δ=4.12-4.06 (6H, m), 2.51-2.50 (2H, t, J=4), 2.43-2.32 (14H, m), 2.22 (3H, s), 1.83-1.79 (2H, m), 1.62-1.60 (2H, m), 1.43 (6H, m), 1.37-1.27 (62H, m), 0.91-0.87 (9H, t, J=8). MS (m/z): [M+H]⁺ calcd. for C₅₀H₁₀₇N₃O₄P, 844.80, found, 844.80.

PL11, yield 33%. ¹H NMR (400 MHz, CDCl₃) δ=4.15-4.06 (6H, m), 2.53-2.50 (2H, t, J=4), 2.44-2.40 (9H, m), 2.37-2.32 (5H, m), 2.22 (3H, s), 1.83-1.79 (2H, m), 1.71-1.68 (1H, m), 1.64-1.60 (4H, m), 1.43 (6H, m), 1.37-1.27 (66H, m), 0.91-0.87 (9H, t, J=8). MS (m/z): [M+H]⁺ calcd. for C₅₃H₁₁₃N₃O₄P, 886.85, found, 886.85.

PL12, yield 32%. ¹H NMR (400 MHz, CDCl₃) δ=4.15-4.06 (6H, m), 2.52-2.31 (22H, m), 1.84-1.77 (2H, m), 1.65-1.60 (4H, m), 1.42-1.41 (6H, m), 1.37-1.27 (49H, m), 0.91-0.87 (9H, t, J=8). MS (m/z): [M+H]⁺ calcd. for C₄₇H₁₀₀N₄O₄P, 815.75, found, 815.75.

PL13, yield 30%. ¹H NMR (400 MHz, CDCl₃) δ=4.16-4.06 (6H, m), 2.52-2.32 (22H, m), 1.82-1.79 (2H, m), 1.66-1.60 (4H, m), 1.42-1.41 (6H, m), 1.37-1.27 (55H, m), 0.91-0.87 (9H, t, J=8). MS (m/z): [M+H]⁺ calcd. for C₅₀H₁₀₆N₄O₄P, 857.80, found, 857.79.

PL14, yield 36%. ¹H NMR (400 MHz, CDCl₃) δ=4.16-4.07 (6H, m), 2.53-2.32 (22H, m), 1.83-1.80 (2H, m), 1.66-1.61 (4H, m), 1.42 (6H, m), 1.37-1.28 (61H, m), 0.91-0.88 (9H, t, J=8). MS (m/z): [M+H]⁺ calcd. for C₅₃H₁₁₂N₄O₄P, 899.84, found, 899.84.

PL15, yield 21%. ¹H NMR (400 MHz, CDCl₃) δ=4.13-4.08 (6H, m), 2.53-2.33 (24H, m), 1.85-1.80 (4H, m), 1.66-1.63 (5H, m), 1.42 (9H, m), 1.38-1.28 (72H, m), 0.91-0.88 (9H, t, J=8). MS (m/z): [M+H]⁺ calcd. for C₅₆H₁₁₈N₄O₄P, 941.89, found, 941.89.

PL16, yield 23%. ¹H NMR (400 MHz, CDCl₃) δ=5.69-5.63 (3H, m), 5.57-5.51 (3H, m), 4.65-4.63 (6H, d, J=8), 4.16-4.08 (6H, m), 2.55-2.40 (10H, m), 2.34-2.30 (6H, m), 2.14-2.09 (6H, m), 1.85-1.80 (2H, m), 1.65-1.62 (9H, m), 1.42 (9H, m), 1.38-1.31 (62H, m), 0.92-0.89 (9H, t, J=8). MS (m/z): [M+H]⁺ calcd. for C₆₄H₁₂₂N₂O₁₀P, 1109.87, found, 1109.89. PL17, yield 23%. ¹H NMR (400 MHz, CDCl₃) δ=5.41-5.28 (12H, m), 4.15-4.06 (6H, d, J=8), 3.15-3.03 (2H, m), 2.97-2.89 (7H, m), 2.78-2.75 (7H, m), 2.07-2.00 (22H, m), 1.62-1.55 (5H, m), 1.35-1.29 (51H, m), 0.90-0.86 (9H, t, J=8). MS (m/z): [M+H]⁺ calcd. for C₆₄H₁₂₂N₂O₄P, 1013.91, found, 1013.91.

PL18, yield 24%. ¹H NMR (400 MHz, CDCl₃) δ=4.22-4.10 (7H, m), 2.43-2.39 (11H, m), 2.34-2.30 (2H, t, J=8), 2.04-2.01 (2H, t, J=8), 1.67-1.63 (4H, m), 1.38-1.28 (71H, m), 0.91-0.88 (9H, t, J=8). MS (m/z): [M+H]⁺ calcd. for C₅₂H₁₀₇N₂O₆P, 887.79, found, 887.79.

GL1, yield 26%. ¹H NMR (400 MHz, CDCl₃) δ=4.17-4.11 (2H, m), 2.70-2.57 (11H, m), 2.33-2.29 (2H, t, J=8), 1.78 (2H, s), 1.69-1.62 (3H, m), 1.54-1.48 (8H, m), 1.28 (59H, s), 0.92-0.88 (9H, t, J=8). MS (m/z): [M+H]⁺ calcd. for C₅₀H₉₅N₂O₁₀, 883.70; found, 883.70. GL2, yield 35%. ¹H NMR (400 MHz, CDCl₃) δ=5.40 (1H, m), 5.24-5.19 (1H, m), 5.04-5.00 (1H, m), 4.48-4.46 (1H, d, J=8), 4.17 (2H, m), 3.91 (2H, m), 3.54-3.51 (1H, m), 2.40-2.38 (12H, m), 2.16 (3H, s), 2.06 (6H, s), 1.99 (4H, s), 1.74 (2H, m), 1.73-1.70 (2H, m), 1.57 (6H, m), 1.27 (52H, s), 0.89 (9H, t, J=8). MS (m/z): [M+H]⁺ calcd. for C₅₃H₁₀₁N₂O₁₀, 925.75, found, 925.74.

GL3, yield 64%. ¹H NMR (400 MHz, CDCl₃) δ=5.41-5.40 (1H, m), 5.32-5.20 (1H, m), 5.04-5.01 (1H, m), 4.48-4.46 (1H, d, J=8), 4.22-4.13 (2H, m), 3.94-3.90 (2H, m), 3.54-3.52 (1H, m), 2.46-2.37 (12H, m), 2.16 (3H, s), 2.06 (6H, s), 2.00 (4H, s), 1.73-1.72 (2H, m), 1.58-1.56 (2H, m), 1.42 (6H, m), 1.28 (55H, s), 0.89 (9H, t, J=8). MS (m/z): [M+H]⁺ calcd. for C₅₆H₁₀₇N₂O₁₀, 967.79, found, 967.79.

GL4, yield 35%. ¹H NMR (400 MHz, CDCl₃) δ=5.39 (1H, m), 5.19-5.15 (1H, m), 5.03-5.01 (1H, m), 4.47-4.45 (1H, m), 4.15-4.14 (2H, m), 3.93-3.92 (2H, m), 3.53-3.51 (1H, m), 2.84-2.74 (6H, m), 2.64-2.59 (4H, m), 2.55-2.51 (2H, m), 2.10 (3H, s), 2.05 (6H, s), 1.98 (6H, s), 1.83-1.78 (4H, m), 1.58 (4H, m), 1.46 (2H, m), 1.26 (63H, s), 0.89-0.88 (9H, t, J=4). MS (m/z): [M+H]⁺ calcd. for C₅₉H₁₁₃N₂O₁₀, 1009.84, found, 1009.84.

GL5, yield 35%. ¹H NMR (400 MHz, CDCl₃) δ=5.41-5.40 (1H, m), 5.22-5.19 (1H, m), 5.04 (1H, m), 4.48-4.46 (1H, d, J=8), 4.17 (2H, m), 3.91 (2H, m), 3.54-3.52 (1H, m), 2.84-2.51 (15H, m), 2.10 (3H, s), 2.16 (3H, s), 2.07 (5H, s), 2.00 (4H, s), 1.73-1.72 (2H, m), 1.62-1.60 (4H, s), 1.43-1.42 (6H, m), 1.26 (53H, s), 0.89-0.88 (9H, t, J=8). MS (m/z): [M+H]⁺ calcd. for C₆₀H₁₁₆N₃O₁₀, 1038.87, found, 1038.86.

GL6, yield 35%. ¹H NMR (400 MHz, CDCl₃) δ=5.41-5.40 (1H, m), 5.24-5.21 (1H, m), 5.04=5.01 (1H, m), 4.48-4.46 (1H, d, J=8), 4.22-4.12 (2H, m), 3.95-3.89 (2H, m), 3.56-3.50 (1H, m), 2.84-2.33 (22H, m), 2.16 (3H, s), 2.07-2.06 (1H, s), 2.00 (3H, s), 1.73-1.63 (6H, m), 1.42 (6H, m), 1.27 (53H, s), 0.91-0.88 (9H, t, J=8). MS (m/z): [M+H]⁺ calcd. for C₆₃H₁₂₁N₄O₁₀, 1093.91, found, 1093.91.

GL7, yield 30%. ¹H NMR (400 MHz, CDCl₃) δ=5.24-5.19 (1H, m), 5.13-5.08 (1H, m), 5.02-4.98 (1H, m), 4.52-4.50 (1H, d, J=8), 4.32-4.27 (1H, m), 4.16-4.13 (1H, m), 3.94-3.89 (1H, m), 3.72-3.69 (1H, m), 3.56-3.50 (1H, m), 3.37-3.34 (1H, m), 2.46-2.35 (15H, m), 2.23 (3H, s), 2.10-2.02 (11H, m), 1.72-1.60 (8H, m), 1.45-1.28 (64H, s), 0.91-0.88 (12H, t, J=8). MS (m/z): [M+H]⁺ calcd. for C₆₀H₁₁₆N₃O₁₀, 1038.87, found, 1038.87.

GL8, yield 65%. ¹H NMR (400 MHz, CDCl₃) δ=5.24-5.19 (1H, m), 5.12-5.08 (1H, m), 5.01-4.97 (1H, m), 4.51-4.49 (1H, d, J=8), 4.31-4.27 (1H, m), 4.16-4.13 (1H, m), 3.92-3.88 (1H, m), 3.70-3.69 (3H, m), 3.55-3.50 (1H, m), 2.47-2.34 (25H, m), 2.10 (3H, s), 2.05-2.02 (9H, m), 1.70 (10H, m), 1.50 (10H, m), 1.27 (55H, s), 0.91-0.88 (9H, t, J=8). MS (m/z): [M+H]⁺ calcd. for C₆₃H₁₂₁N₄O₁₀, 1093.91, found, 1093. 91.

GL9, yield 49%. ¹H NMR (400 MHz, CDCl₃) δ=5.69-5.63 (1H, m), 5.57-5.51 (3H, m), 5.24-5.19 (1H, m), 5.12-5.08 (1H, m), 5.02-4.97 (1H, m), 4.64-4.63 (6H, d, J=4), 4.52-4.50 (1H, d, J=8), 4.31-4.27 (1H, m), 4.17-4.11 (1H, m), 3.94-3.88 (1H, m), 3.73-3.69 (1H, m), 3.54-3.51 (1H, m), 2.47-2.30 (18H, m), 2.14-2.02 (17H, m), 1.65-1.62 (11H, m), 1.40-1.31 (55H, m), 0.92-0.88 (9H, t, J=8). MS (m/z): [M+H]⁺ calcd. for C₇₄H₁₃₁N₂O₁₆, 1303.95, found, 1303.94.

GL10, yield 33%. ¹H NMR (400 MHz, CDCl₃) δ=5.64-5.63 (2H, m), 5.57-5.51 (2H, m), 5.24-5.19 (1H, m), 5.13-5.08 (1H, m), 5.02-4.97 (1H, m), 4.64-4.63 (4H, d, J=4), 4.52-4.50 (1H, d, J=8), 4.31-4.27 (1H, m), 4.17-4.13 (1H, m), 3.95-3.89 (1H, m), 3.72-3.70 (2H, m), 3.58-3.52 (2H, m), 2.55-2.38 (9H, m), 2.34-2.30 (5H, m), 2.22 (2H, m), 2.16-2.02 (16H, m), 1.79-1.60 (9H, m), 1.46-1.27 (32H, m), 0.92-0.88 (6H, t, J=8). MS (m/z): [M+H]⁺ calcd. for C₅₇H₁₀₁N₂O₁₄, 1037.73, found, 1037.73.

GL11, yield 20%. ¹H NMR (400 MHz, CDCl₃) δ=5.25-5.20 (1H, m), 5.14-5.08 (1H, m), 5.02-4.97 (1H, m), 4.55-4.53 (1H, d, J=8), 4.32-4.28 (1H, m), 4.17-4.13 (1H, m), 4.08-4.05 (6H, t, J=8), 3.93-3.88 (1H, m), 3.75-3.71 (1H, s), 3.57-3.53 (1H, m), 2.80-2.76 (6H, t, J=8), 2.47-2.43 (10H, m), 2.11 (3H, s), 2.07 (3H, s), 2.04 (3H, s), 2.02 (3H, s), 1.67-1.60 (12H, m), 1.32-1.28 (54H, m), 0.92-0.88 (9H, t, J=8). MS (m/z): [M+H]⁺ calcd. for C₆₅H₁₁₉N₂O₁₆, 1183.86, found, 1183.85.

GL12, yield 63%. ¹H NMR (400 MHz, CDCl₃) δ=5.24-5.19 (1H, m), 5.12-5.08 (1H, m), 5.02-4.97 (1H, m), 4.52-4.50 (1H, d, J=8), 4.31-4.26 (1H, m), 4.16-4.12 (1H, m), 3.94-3.89 (1H, m), 3.71-3.68 (2H, t, J=8), 3.58-3.52 (1H, m), 2.44-2.33 (12H, t, J=8), 2.21 (3H, s), 2.10 (3H, s), 2.06 (3H, s), 2.04 (3H, s), 2.02 (3H, s), 1.75-1.63 (6H, m), 1.44 (4H, m), 1.27 (37H, m), 0.91-0.87 (6H, t, J=8). MS (m/z): [M+H]⁺ calcd. for C₄₅H₈₅N₂O₁₀, 813.62, found, 813.62.

GL13, yield 44%. ¹H NMR (400 MHz, CDCl₃) δ=5.42 (1H, m), 5.24-5.19 (1H, m), 5.13-5.09 (1H, m), 4.52-4.50 (1H, m), 4.21-4.17 (1H, m), 4.07-3.87 (3H, m), 3.53-3.47 (1H, m), 2.52-2.39 (10H, m), 1.77-1.66 (4H, m), 1.50-1.42 (5H, m), 1.27-1.13 (89H, m), 0.91-0.87 (9H, t, J=8). MS (m/z): [M+H]⁺ calcd. for C₆₈H₁₃₁N₂O₁₀, 1135.98, found, 1135.98.

GL14, yield 37%. ¹H NMR (400 MHz, CDCl₃) δ=5.43-5.24 (4H, m), 5.09-5.04 (1H, t, J=8), 4.89-4.82 (2H, m), 4.53-4.47 (2H, m), 4.29-4.22 (3H, m), 4.07-3.96 (4H, m), 3.90-3.87 (1H, m), 3.72-3.67 (4H, m), 3.53-3.50 (1H, m), 2.48-2.37 (10H, m), 2.16-2.01 (27H, m), 1.73-1.69 (3H, m), 1.45-1.41 (6H, m), 1.27 (45H, s), 0.91-0.87 (9H, t, J=8). MS (m/z): [M+H]^{P} calcd. for C₆₈H₁₂₃N₂O₁₈, 1255.88, found, 1255.88.

GL15, yield 48%. ¹H NMR (400 MHz, CDCl₃) δ=5.36-5.35 (1H, d, J=4), 5.22-5.18 (1H, t, J=8), 5.14-5.10 (1H, m), 4.98-4.95 (1H, m), 4.91-4.87 (1H, m), 4.50-4.45 (3H, m), 4.15-4.07 (3H, m), 3.88-3.78 (4H, m), 3.62-3.58 (1H, m), 3.52-3.47 (1H, m), 2.39-2.37 (1H, m), 2.16 (3H, s), 2.13 (3H, s), 2.07-2.04 (12H, m), 1.97 (3H, s), 1.71-1.68 (3H, m), 1.55-1.53 (2H, m), 1.41 (6H, m), 1.27 (51H, m), 0.91-0.87 (9H, t, J=8). MS (m/z): [M+H]⁺ calcd. for C₆₈H₁₂₃N₂O₁₈, 1255.88, found, 1255.88.

GL16, yield 55%. ¹H NMR (400 MHz, CDCl₃) δ=5.34-5.30 (1H, m), 5.24-5.23 (1H, m), 4.99 (1H, m), 4.35-4.28 (2H, m), 4.14-4.10 (1H, m), 3.75-3.71 (1H, t, J=8), 3.45-3.41 (1H, t, J=8), 2.41 (12H, m), 2.12-2.05 (9H, m), 1.70 (3H, m), 1.58 (3H, m), 1.42 (6H, m), 1.27 (52H, s), 0.91-0.87 (9H, t, J=8). MS (m/z): [M+H]⁺ calcd. for C₅₃H₁₀₃N₂O₈, 895.77, found, 895.77.

SEQUENCES
RNA sequences
Human OX40 mRNA(With 5′UTR and 3′UTR)
(SEQ ID NO: 1)
GGGAAAAGUAGAAAGAAAGAAAGAAGAGAAAAUAAAGACAAAGAGCCA
CCAUGUGCGUGGGAGCACGGAGACUGGGAAGGGGACCUUGCGCCGCCC
UGCUGCUGCUGGGCCUGGGCCUGUCCACCGUGACAGGCCUGCACUGCG
UGGGCGACACCUACCCUUCUAACGAUAGGUGCUGUCACGAGUGUCGCC
CAGGCAAUGGCAUGGUGUCCAGGUGCUCCCGCUCUCAGAACACCGUGU
GCCGGCCUUGUGGCCCAGGCUUCUAUAAUGACGUGGUGAGCUCCAAGC
CCUGCAAGCCUUGUACAUGGUGCAACCUGCGGAGCGGCUCCGAGAGAA
AGCAGCUGUGCACCGCCACACAGGAUACCGUGUGCCGGUGUAGAGCCG
GCACACAGCCACUGGACUCUUACAAGCCAGGAGUGGAUUGUGCACCUU
GCCCACCUGGCCACUUUAGCCCAGGCGACAACCAGGCCUGUAAGCCCU
GGACCAAUUGCACACUGGCAGGCAAGCACACCCUGCAGCCAGCAUCUA
AUUCUAGCGAUGCCAUCUGCGAGGACAGAGAUCCACCAGCAACCCAGC
CUCAGGAGACACAGGGACCUCCAGCCAGGCCAAUCACCGUGCAGCCAA
CAGAGGCAUGGCCUCGGACCUCUCAGGGACCAAGCACAAGACCCGUGG
AGGUGCCUGGAGGAAGGGCAGUGGCAGCUAUCUUGGGGCUCGGGUUGG
UACUGGGACUGCUUGGCCCACUUGCUAUCUUGCUGGCUCUGUAUCUGC
UGAGGCGCGACCAGCGCCUGCCCCCUGAUGCACACAAGCCACCAGGAG
GAGGAAGCUUCCGGACCCCAAUCCAGGAGGAGCAGGCAGACGCACACU
CCACACUGGCCAAGAUCUGAUUGUGUAUGCGUUAAUAAAAAGAAGGAA
CUCGUA
Mouse OX40 mRNA(With 5′UTR and 3′UTR)
(SEQ ID NO: 2)
GGGAAAAGUAGAAAGAAAGAAAGAAGAGAAAAUAAAGACAAAGAGCCA
CCAUGUAUGUGUGGGUUCAGCAGCCCACAGCCCUUCUGCUGCUGGGAC
UCACACUUGGAGUUACAGCAAGGCGGCUCAACUGUGUUAAACAUACCU
ACCCCAGUGGUCACAAGUGCUGUCGUGAGUGCCAGCCAGGCCAUGGUA
UGGUGAGCCGCUGUGAUCAUACCAGGGAUACUCUAUGUCAUCCGUGUG
AGACUGGCUUCUACAAUGAAGCUGUCAAUUAUGAUACCUGCAAGCAGU
GUACACAGUGCAACCAUCGAAGUGGAAGUGAACUCAAGCAGAAUUGCA
CACCUACUCAGGAUACUGUCUGCAGAUGUAGACCAGGCACCCAACCUC
GGCAGGACAGCGGCUACAAGCUUGGAGUUGACUGUGUUCCCUGCCCUC
CUGGCCACUUUUCUCCAGGCAACAACCAGGCCUGCAAGCCCUGGACCA
AUUGUACCUUAUCUGGAAAGCAGACCCGCCACCCAGCCAGUGACAGCU
UGGACGCAGUCUGUGAGGACAGAAGCCUCCUGGCCACACUGCUCUGGG
AGACCCAGCGCCCUACAUUCAGGCCAACCACUGUCCAAUCCACCACAG
UCUGGCCCAGGACUUCUGAGUUGCCCUCUCCACCCACCUUGGUGACUC
CUGAGGGCCCUGCAUUUGCUGUUCUCCUAGGCCUGGGCCUGGGCCUGC
UGGCUCCCUUGACUGUCCUGCUGGCCUUGUACCUGCUCCGGAAGGCUU
GGAGAUUGCCUAACACUCCCAAACCUUGUUGGGGAAACAGCUUCAGGA
CCCCGAUCCAGGAGGAACACACAGACGCACACUUUACUCUGGCCAAGA
UCUGAUUGUGUAUGCGUUAAUAAAAAGAAGGAACUCGUA
HETEROLOGOUS 5′UTR/3′UTR
5′UTR:
(SEQ ID NO: 3)
GGGAAAAGUAGAAAGAAAGAAAGAAGAGAAAAUAAAGACAAAGAGCCA
CC
(SEQ ID NO: 4)
3′UTR: UUGUGUAUGCGUUAAUAAAAAGAAGGAACUCGUA
Human OX40 coding sequence
(SEQ ID NO: 5)
AUGUGCGUGGGAGCACGGAGACUGGGAAGGGGACCUUGCGCCGCCCUG
CUGCUGCUGGGCCUGGGCCUGUCCACCGUGACAGGCCUGCACUGCGUG
GGCGACACCUACCCUUCUAACGAUAGGUGCUGUCACGAGUGUCGCCCA
GGCAAUGGCAUGGUGUCCAGGUGCUCCCGCUCUCAGAACACCGUGUGC
CGGCCUUGUGGCCCAGGCUUCUAUAAUGACGUGGUGAGCUCCAAGCCC
UGCAAGCCUUGUACAUGGUGCAACCUGCGGAGCGGCUCCGAGAGAAAG
CAGCUGUGCACCGCCACACAGGAUACCGUGUGCCGGUGUAGAGCCGGC
ACACAGCCACUGGACUCUUACAAGCCAGGAGUGGAUUGUGCACCUUGC
CCACCUGGCCACUUUAGCCCAGGCGACAACCAGGCCUGUAAGCCCUGG
ACCAAUUGCACACUGGCAGGCAAGCACACCCUGCAGCCAGCAUCUAAU
UCUAGCGAUGCCAUCUGCGAGGACAGAGAUCCACCAGCAACCCAGCCU
CAGGAGACACAGGGACCUCCAGCCAGGCCAAUCACCGUGCAGCCAACA
GAGGCAUGGCCUCGGACCUCUCAGGGACCAAGCACAAGACCCGUGGAG
GUGCCUGGAGGAAGGGCAGUGGCAGCUAUCUUGGGGCUCGGGUUGGUA
CUGGGACUGCUUGGCCCACUUGCUAUCUUGCUGGCUCUGUAUCUGCUG
AGGCGCGACCAGCGCCUGCCCCCUGAUGCACACAAGCCACCAGGAGGA
GGAAGCUUCCGGACCCCAAUCCAGGAGGAGCAGGCAGACGCACACUCC
ACACUGGCCAAGAUCUGA
Mouse OX40 coding sequence
(SEQ ID NO: 6)
AUGUAUGUGUGGGUUCAGCAGCCCACAGCCCUUCUGCUGCUGGGACUC
ACACUUGGAGUUACAGCAAGGCGGCUCAACUGUGUUAAACAUACCUAC
CCCAGUGGUCACAAGUGCUGUCGUGAGUGCCAGCCAGGCCAUGGUAUG
GUGAGCCGCUGUGAUCAUACCAGGGAUACUCUAUGUCAUCCGUGUGAG
ACUGGCUUCUACAAUGAAGCUGUCAAUUAUGAUACCUGCAAGCAGUGU
ACACAGUGCAACCAUCGAAGUGGAAGUGAACUCAAGCAGAAUUGCACA
CCUACUCAGGAUACUGUCUGCAGAUGUAGACCAGGCACCCAACCUCGG
CAGGACAGCGGCUACAAGCUUGGAGUUGACUGUGUUCCCUGCCCUCCU
GGCCACUUUUCUCCAGGCAACAACCAGGCCUGCAAGCCCUGGACCAAU
UGUACCUUAUCUGGAAAGCAGACCCGCCACCCAGCCAGUGACAGCUUG
GACGCAGUCUGUGAGGACAGAAGCCUCCUGGCCACACUGCUCUGGGAG
ACCCAGCGCCCUACAUUCAGGCCAACCACUGUCCAAUCCACCACAGUC
UGGCCCAGGACUUCUGAGUUGCCCUCUCCACCCACCUUGGUGACUCCU
GAGGGCCCUGCAUUUGCUGUUCUCCUAGGCCUGGGCCUGGGCCUGCUG
GCUCCCUUGACUGUCCUGCUGGCCUUGUACCUGCUCCGGAAGGCUUGG
AGAUUGCCUAACACUCCCAAACCUUGUUGGGGAAACAGCUUCAGGACC
CCGAUCCAGGAGGAACACACAGACGCACACUUUACUCUGGCCAAGAUC
UGA
DNA sequences
Human OX40 mRNA(With 5′UTR and 3′UTR)
(SEQ ID NO: 7)
GGGAAAAGTAGAAAGAAAGAAAGAAGAGAAAATAAAGACAAAGAGCCA
CCATGTGCGTGGGAGCACGGAGACTGGGAAGGGGACCTTGCGCCGCCC
TGCTGCTGCTGGGCCTGGGCCTGTCCACCGTGACAGGCCTGCACTGCG
TGGGCGACACCTACCCTTCTAACGATAGGTGCTGTCACGAGTGTCGCC
CAGGCAATGGCATGGTGTCCAGGTGCTCCCGCTCTCAGAACACCGTGT
GCCGGCCTTGTGGCCCAGGCTTCTATAATGACGTGGTGAGCTCCAAGC
CCTGCAAGCCTTGTACATGGTGCAACCTGCGGAGCGGCTCCGAGAGAA
AGCAGCTGTGCACCGCCACACAGGATACCGTGTGCCGGTGTAGAGCCG
GCACACAGCCACTGGACTCTTACAAGCCAGGAGTGGATTGTGCACCTT
GCCCACCTGGCCACTTTAGCCCAGGCGACAACCAGGCCTGTAAGCCCT
GGACCAATTGCACACTGGCAGGCAAGCACACCCTGCAGCCAGCATCTA
ATTCTAGCGATGCCATCTGCGAGGACAGAGATCCACCAGCAACCCAGC
CTCAGGAGACACAGGGACCTCCAGCCAGGCCAATCACCGTGCAGCCAA
CAGAGGCATGGCCTCGGACCTCTCAGGGACCAAGCACAAGACCCGTGG
AGGTGCCTGGAGGAAGGGCAGTGGCAGCTATCTTGGGGCTCGGGTTGG
TACTGGGACTGCTTGGCCCACTTGCTATCTTGCTGGCTCTGTATCTGC
TGAGGCGCGACCAGCGCCTGCCCCCTGATGCACACAAGCCACCAGGAG
GAGGAAGCTTCCGGACCCCAATCCAGGAGGAGCAGGCAGACGCACACT
CCACACTGGCCAAGATCTGATTGTGTATGCGTTAATAAAAAGAAGGAA
CTCGTA
Mouse OX40 mRNA(With 5′UTR and 3′UTR)
(SEQ ID NO: 8)
GGGAAAAGTAGAAAGAAAGAAAGAAGAGAAAATAAAGACAAAGAGCCA
CCATGTATGTGTGGGTTCAGCAGCCCACAGCCCTTCTGCTGCTGGGAC
TCACACTTGGAGTTACAGCAAGGCGGCTCAACTGTGTTAAACATACCT
ACCCCAGTGGTCACAAGTGCTGTCGTGAGTGCCAGCCAGGCCATGGTA
TGGTGAGCCGCTGTGATCATACCAGGGATACTCTATGTCATCCGTGTG
AGACTGGCTTCTACAATGAAGCTGTCAATTATGATACCTGCAAGCAGT
GTACACAGTGCAACCATCGAAGTGGAAGTGAACTCAAGCAGAATTGCA
CACCTACTCAGGATACTGTCTGCAGATGTAGACCAGGCACCCAACCTC
GGCAGGACAGCGGCTACAAGCTTGGAGTTGACTGTGTTCCCTGCCCTC
CTGGCCACTTTTCTCCAGGCAACAACCAGGCCTGCAAGCCCTGGACCA
ATTGTACCTTATCTGGAAAGCAGACCCGCCACCCAGCCAGTGACAGCT
TGGACGCAGTCTGTGAGGACAGAAGCCTCCTGGCCACACTGCTCTGGG
AGACCCAGCGCCCTACATTCAGGCCAACCACTGTCCAATCCACCACAG
TCTGGCCCAGGACTTCTGAGTTGCCCTCTCCACCCACCTTGGTGACTC
CTGAGGGCCCTGCATTTGCTGTTCTCCTAGGCCTGGGCCTGGGCCTGC
TGGCTCCCTTGACTGTCCTGCTGGCCTTGTACCTGCTCCGGAAGGCTT
GGAGATTGCCTAACACTCCCAAACCTTGTTGGGGAAACAGCTTCAGGA
CCCCGATCCAGGAGGAACACACAGACGCACACTTTACTCTGGCCAAGA
TCTGATTGTGTATGCGTTAATAAAAAGAAGGAACTCGTA
HETEROLOGOUS 5′UTR/3′UTR
5′UTR:
(SEQ ID NO: 9)
GGGAAAAGTAGAAAGAAAGAAAGAAGAGAAAATAAAGACAAAGAGCCA
CC
3′UTR:
(SEQ ID NO: 10)
TTGTGTATGCGTTAATAAAAAGAAGGAACTCGTA
Human OX40 coding sequence
(SEQ ID NO: 11)
ATGTGCGTGGGAGCACGGAGACTGGGAAGGGGACCTTGCGCCGCCCTG
CTGCTGCTGGGCCTGGGCCTGTCCACCGTGACAGGCCTGCACTGCGTG
GGCGACACCTACCCTTCTAACGATAGGTGCTGTCACGAGTGTCGCCCA
GGCAATGGCATGGTGTCCAGGTGCTCCCGCTCTCAGAACACCGTGTGC
CGGCCTTGTGGCCCAGGCTTCTATAATGACGTGGTGAGCTCCAAGCCC
TGCAAGCCTTGTACATGGTGCAACCTGCGGAGCGGCTCCGAGAGAAAG
CAGCTGTGCACCGCCACACAGGATACCGTGTGCCGGTGTAGAGCCGGC
ACACAGCCACTGGACTCTTACAAGCCAGGAGTGGATTGTGCACCTTGC
CCACCTGGCCACTTTAGCCCAGGCGACAACCAGGCCTGTAAGCCCTGG
ACCAATTGCACACTGGCAGGCAAGCACACCCTGCAGCCAGCATCTAAT
TCTAGCGATGCCATCTGCGAGGACAGAGATCCACCAGCAACCCAGCCT
CAGGAGACACAGGGACCTCCAGCCAGGCCAATCACCGTGCAGCCAACA
GAGGCATGGCCTCGGACCTCTCAGGGACCAAGCACAAGACCCGTGGAG
GTGCCTGGAGGAAGGGCAGTGGCAGCTATCTTGGGGCTCGGGTTGGTA
CTGGGACTGCTTGGCCCACTTGCTATCTTGCTGGCTCTGTATCTGCTG
AGGCGCGACCAGCGCCTGCCCCCTGATGCACACAAGCCACCAGGAGGA
GGAAGCTTCCGGACCCCAATCCAGGAGGAGCAGGCAGACGCACACTCC
ACACTGGCCAAGATCTGA
Mouse OX40 coding sequence
(SEQ ID NO: 12)
ATGTATGTGTGGGTTCAGCAGCCCACAGCCCTTCTGCTGCTGGGACTC
ACACTTGGAGTTACAGCAAGGCGGCTCAACTGTGTTAAACATACCTAC
CCCAGTGGTCACAAGTGCTGTCGTGAGTGCCAGCCAGGCCATGGTATG
GTGAGCCGCTGTGATCATACCAGGGATACTCTATGTCATCCGTGTGAG
ACTGGCTTCTACAATGAAGCTGTCAATTATGATACCTGCAAGCAGTGT
ACACAGTGCAACCATCGAAGTGGAAGTGAACTCAAGCAGAATTGCACA
CCTACTCAGGATACTGTCTGCAGATGTAGACCAGGCACCCAACCTCGG
CAGGACAGCGGCTACAAGCTTGGAGTTGACTGTGTTCCCTGCCCTCCT
GGCCACTTTTCTCCAGGCAACAACCAGGCCTGCAAGCCCTGGACCAAT
TGTACCTTATCTGGAAAGCAGACCCGCCACCCAGCCAGTGACAGCTTG
GACGCAGTCTGTGAGGACAGAAGCCTCCTGGCCACACTGCTCTGGGAG
ACCCAGCGCCCTACATTCAGGCCAACCACTGTCCAATCCACCACAGTC
TGGCCCAGGACTTCTGAGTTGCCCTCTCCACCCACCTTGGTGACTCCT
GAGGGCCCTGCATTTGCTGTTCTCCTAGGCCTGGGCCTGGGCCTGCTG
GCTCCCTTGACTGTCCTGCTGGCCTTGTACCTGCTCCGGAAGGCTTGG
AGATTGCCTAACACTCCCAAACCTTGTTGGGGAAACAGCTTCAGGACC
CCGATCCAGGAGGAACACACAGACGCACACTTTACTCTGGCCAAGATC
TGA
Coding sequence of mouse OX40 ligand.
(SEQ ID NO: 13)
ATGGAAGGGGAAGGGGTTCAACCCCTGGATGAGAATCTGGAAAACGGA
TCAAGGCCAAGATTCAAGTGGAAGAAGACGCTAAGGCTGGTGGTCTCT
GGGATCAAGGGAGCAGGGATGCTTCTGTGCTTCATCTATGTCTGCCTG
CAACTCTCTTCCTCTCCGGCAAAGGACCCTCCAATCCAAAGACTCAGA
GGAGCAGTTACCAGATGTGAGGATGGGCAACTATTCATCAGCTCATAC
AAGAATGAGTATCAAACTATGGAGGTGCAGAACAATTCGGTTGTCATC
AAGTGCGATGGGCTTTATATCATCTACCTGAAGGGCTCCTTTTTCCAG
GAGGTCAAGATTGACCTTCATTTCCGGGAGGATCATAATCCCATCTCT
ATTCCAATGCTGAACGATGGTCGAAGGATTGTCTTCACTGTGGTGGCC
TCTTTGGCTTTCAAAGATAAAGTTTACCTGACTGTAAATGCTCCTGAT
ACTCTCTGCGAACACCTCCAGATAAATGATGGGGAGCTGATTGTTGTC
CAGCTAACGCCTGGATACTGTGCTCCTGAAGGATCTTACCACAGCACT
GTGAACCAAGTACCACTGTGA
Coding sequence of human OX40 ligand
(SEQ ID: 14)
ATGGAAAGGGTCCAACCCCTGGAAGAGAATGTGGGAAATGCAGCCAGG
CCAAGATTCGAGAGGAACAAGCTATTGCTGGTGGCCTCTGTAATTCAG
GGACTGGGGCTGCTCCTGTGCTTCACCTACATCTGCCTGCACTTCTCT
GCTCTTCAGGTATCACATCGGTATCCTCGAATTCAAAGTATCAAAGTA
CAATTTACCGAATATAAGAAGGAGAAAGGTTTCATCCTCACTTCCCAA
AAGGAGGATGAAATCATGAAGGTGCAGAACAACTCAGTCATCATCAAC
TGTGATGGGTTTTATCTCATCTCCCTGAAGGGCTACTTCTCCCAGGAA
GTCAACATTAGCCTTCATTACCAGAAGGATGAGGAGCCCCTCTTCCAA
CTGAAGAAGGTCAGGTCTGTCAACTCCTTGATGGTGGCCTCTCTGACT
TACAAAGACAAAGTCTACTTGAATGTGACCACTGACAATACCTCCCTG
GATGACTTCCATGTGAATGGCGGAGAACTGATTCTTATCCATCAAAAT
CCTGGTGAATTCTGTGTCCTTTGA
Coding sequence of mouse ICOS
(SEQ ID NO: 15)
ATGAA GCCGTACTTCTGCCGTGTCT TTGTCTTCTG CTTCCTAATC
AGACTTTTAA CAGGAGAAAT CAATGGCTCGGCCGATCATA
GGATGTTTTC ATTTCACAAT GGAGGTGTAC AGATTTCTTG
TAAATACCCTGAGACTGTCC AGCAGTTAAA AATGCGATTG
TTCAGAGAGA GAGAAGTCCT CTGCGAACTCACCAAGACCA
AGGGAAGCGG AAATGCGGTG TCCATCAAGA ATCCAATGCT
CTGTCTATATCATCTGTCAA ACAACAGCGT CTCTTTTTTC
CTAAACAACC CAGACAGCTC CCAGGGAAGCTATTACTTCT
GCAGCCTGTC CATTTTTGAC CCACCTCCTT TTCAAGAAAG
GAACCTTAGTGGAGGATATT TGCATATTTA
TGAATCCCAGCTCTGCTGCCAGCTGAAGCTCTGGCTACCCGTAGGGTG
TGCAGCT TTCGT TGTGGTACTC CTTTTTGGAT GCATACTTAT
CATCTGGTTTTCAAAAAAGA AATACGGATCCAGTGTGCATGACCCTA
ATAGTGAATACATGTTCATGGCGGCAGT CAACA CAAACAAAAA
GTCTAGACTT GCAGGTGTGA CCTCATAA
Coding sequence of human ICOS
(SEQ ID NO: 16)
ATG AAGTCAGGCC TCTGGTATTT CTTTCTCTTC TGCTTGCGCA
TTAAAGTTTTAACAGGAGAA ATCAATGGTT CTGCCAATTA
TGAGATGTTT ATATTTCACA ACGGAGGTGT ACAAATTTTA
TGCAAATATC CTGACATTGT CCAGCAATTT AAAATGCAGT
TGCTGAAAGGGGGGCAAATA CTCTGCGATC TCACTAAGAC
AAAAGGAAGT GGAAACACAG TGTCCATTAA GAGTCTGAAA
TTCTGCCATT CTCAGTTATC CAACAACAGT GTCTCTTTTT
TTCTATACAA CTTGGACCAT TCTCATGCCA ACTATTACTT
CTGCAACCTA TCAATTTTTG ATCCTCCTCCTTTTAAAGTA
ACTCTTACAG GAGGATATTT GCATATTTAT GAATCACAAC
TTTGTTGCCAGCTGAAGTTC TGGTTACCCA TAGGATGTGC
AGCCTTTGTT GTAGTCTGCA TTTTGGGATGCATACTTATT
TGTTGGCTTA CAAAAAAGAA GTATTCATCC AGTGTGCACG
ACCCTAACGGTGAATACATG TTCATGAGAG CAGTGAACAC
AGCCAAAAAA TCTAGACTCA CAGATGTGAC CCTATAA
Coding sequence of mouse CD137 (4-1BB)
(SEQ ID NO: 17)
ATGGGAAAC AACTGTTACA ACGTGGTGGT CATTGTGCTG
CTGCTAGTGGGCTGTGAGAA GGTGGGAGCC GTGCAGAACT
CCTGTGATAA CTGTCAGCCT GGTACTTTCTGCAGAAAATA
CAATCCAGTC TGCAAGAGCT GCCCTCCAAG TACCTTCTCC
AGCATAGGTGGACAGCCGAA CTGTAACATC TGCAGAGTGTGT
GCAGGCTA TTTCAGGTTC AAGAAGTTTTGCTCCTCTAC
CCACAACGCG GAGTGTGAGT GCATTGAAGG ATTCCATTGC
TTGGGGCCACAGTGCACCAG ATGTGAAAAG GACTGCAGGC
CTGGCCAGGA GCTAACGAAG CAGGGTTGCAAAACCTGTAG
CTTGGGAACA TTTAATGACC AGAACGGTAC TGGCGTCTGT
CGACCCTGGACGAACTGCTC TCTAGACGGA AGGTCTGTGC
TTAAGACCGG GACCACGGAG AAGGACGTGGTGTGTGGACC
CCCTGTGGTG AGCTTCTCTC CCAGTACCAC CATTTCTGTG
ACTCCAGAGGGAGGACCAGG AGGGCACTCC TTGCAGGTCC
TTACCTTGTT CCTGGCGCTG ACATCGGCTTTGCTGCTGGC
CCTGATCTTC ATTACTCTCC TGTTCTCTGT GCTCAAATGG
ATCAGGAAAA AATTCCCCCA CATATTCAAG CAACCATTTA
AGAAGACCAC TGGAGCAGCT CAAGAGGAAGATGCTTGTAG
CTGCCGATGT CCACAGGAAG AAGAAGGAGG AGGAGGAGGC
TATGAGCTGTGA
Coding sequence of human CD137 (4-1BB)
(SEQ ID NO: 18)
ATGGGAAAC AGCTGTTACA ACATAGTAGC
CACTCTGTTGCTGGTCCTCAACTTTGAGAGGACAAGATCATTGCAGGA
TCCTTGTAGTAACTGCCCAGCTGGTACATTCTGTGATAATAACAGGAA
TCAGATTTGCAGTCCCTGTCCTCCAAATAGTTTCTCCAGCGCAGGTGG
ACAAAGGACCTGTGACATATGCAGGCAGTGTAAAGGTGTTTTCAGGAC
CAGGAAGGAGTGTTCCTCCACCAGCAATGCAGAGTGTGACTGCACTCC
AGGGTTTCACTGCCTGGGGGCAGGATGCAGCATGTGTGAACAGGATTG
TAAACAAGGTCAAGAACTGACAAAAAAAGGTTGTAAAGACTGTTGCTT
TGGGACATTTAACGATCAGAAACGTGGCATCTGTCGACCCTGGACAAA
CTGTTCTTTGGATGGAAAGTCTGTGCTTGTGAATGGGACGAAGGAGAG
GGACGTGGTCTGTGGACCATCTCCAGCCGACCTCTCTCCGGGAGCATC
CTCTGTGACCCCGCCTGCCCCTGCGAGAGAGCCAGGACACTCTCCGCA
GATCATCTCCTTCTTTCTTGCGCTGACGTCGACTGCGTTGCTCTTCCT
GCTGTTCTTCCTCACGCTCCGTTTCTCTGTTGTTAAACGGGGCAGAAA
GAAACTCCTGTATATATTCAAACAACCATTTATGAGACCAGTACAAAC
TACTCAAGAGGAAGATGGCTGTAGCTGCCGATTTCCAGAAGAAGAAGA
AGGAGGATGTGAACTGTGA
Coding sequence of mouse CD137 ligand (4-1BBL)
(SEQ ID NO: 19)
ATGGACCAGCACACACTTGATGTGGAGGATACCGCGGATGCCAGACAT
CCAGCAGGTACTTCGTGCCCCTCGGATGCGGCGCTCCTCAGAGATACC
GGGCTCCTCGCGGACGCTGCGCTCCTCTCAGATACTGTGCGCCCCACA
AATGCCGCGCTCCCCACGGATGCTGCCTACCCTGCGGTTAATGTTCGG
GATCGCGAGGCCGCGTGGCCGCCTGCACTGAACTTCTGTTCCCGCCAC
CCAAAGCTCTATGGCCTAGTCGCTTTGGTTTTGCTGCTTCTGATCGCC
GCCTGTGTTCCTATCTTCACCCGCACCGAGCCTCGGCCAGCGCTCACA
ATCACCACCTCGCCCAACCTGGGTACCCGAGAGAATAATGCAGACCAG
GTCACCCCTGTTTCCCACATTGGCTGCCCCAACACTACACAACAGGGC
TCTCCTGTGTTCGCCAAGCTACTGGCTAAAAACCAAGCATCGTTGTGC
AATACAACTCTGAACTGGCACAGCCAAGATGGAGCTGGGAGCTCATAC
CTATCTCAAGGTCTGAGGTACGAAGAAGACAAAAAGGAGTTGGTGGTA
GACAGTCCCGGGCTCTACTACGTATTTTTGGAACTGAAGCTCAGTCCA
ACATTCACAAACACAGGCCACAAGGTGCAGGGCTGGGTCTCTCTTGTT
TTGCAAGCAAAGCCTCAGGTAGATGACTTTGACAACTTGGCCCTGACA
GTGGAACTGTTCCCTTGCTCCATGGAGAACAAGTTAGTGGACCGTTCC
TGGAGTCAACTGTTGCTCCTGAAGGCTGGCCACCGCCTCAGTGTGGGT
CTGAGGGCTTATCTGCATGGAGCCCAGGATGCATACAGAGACTGGGAG
CTGTCTTATCCCAACACCACCAGCTTTGGACTCTTTCTTGTGAAACCC
GACAACCCATGGGAATGA
Coding sequence of human CD137 ligand (4-1BBL)
(SEQ ID NO: 20)
ATGGAATACGCCTCTGACGCTTCACTGGACCCCGAAGCCCCGTGGCCT
CCCGCGCCCCGCGCTCGCGCCTGCCGCGTACTGCCTTGGGCCCTGGTC
GCGGGGCTGCTGCTGCTGCTGCTGCTCGCTGCCGCCTGCGCCGTCTTC
CTCGCCTGCCCCTGGGCCGTGTCCGGGGCTCGCGCCTCGCCCGGCTCC
GCGGCCAGCCCGAGACTCCGCGAGGGTCCCGAGCTTTCGCCCGACGAT
CCCGCCGGCCTCTTGGACCTGCGGCAGGGCATGTTTGCGCAGCTGGTG
GCCCAAAATGTTCTGCTGATCGATGGGCCCCTGAGCTGGTACAGTGAC
CCAGGCCTGGCAGGCGTGTCCCTGACGGGGGGCCTGAGCTACAAAGAG
GACACGAAGGAGCTGGTGGTGGCCAAGGCTGGAGTCTACTATGTCTTC
TTTCAACTAGAGCTGCGGCGCGTGGTGGCCGGCGAGGGCTCAGGCTCC
GTTTCACTTGCGCTGCACCTGCAGCCACTGCGCTCTGCTGCTGGGGCC
GCCGCCCTGGCTTTGACCGTGGACCTGCCACCCGCCTCCTCCGAGGCT
CGGAACTCGGCCTTCGGTTTCCAGGGCCGCTTGCTGCACCTGAGTGCC
GGCCAGCGCCTGGGCGTCCATCTTCACACTGAGGCCAGGGCACGCCAT
GCCTGGCAGCTTACCCAGGGCGCCACAGTCTTGGGACTCTTCCGGGTG
ACCCCCGAAATCCCAGCCGGACTCCCTTCACCGAGGTCGGAATAA
Coding sequence of mouse GITR
(SEQ ID NO: 21)
ATGGGGGCATGGGCCATGCTGTATGGAGTCTCGATGCTCTGTGTGCTG
GACCTAGGTCAGCCGAGTGTAGTTGAGGAGCCTGGCTGTGGCCCTGGC
AAGGTTCAGAACGGAAGTGGCAACAACACTCGCTGCTGCAGCCTGTAT
GCTCCAGGCAAGGAGGACTGTCCAAAAGAAAGGTGCATATGTGTCACA
CCTGAGTACCACTGTGGAGACCCTCAGTGCAAGATCTGCAAGCACTAC
CCCTGCCAACCAGGCCAGAGGGTGGAGTCTCAAGGGGATATTGTGTTT
GGCTTCCGGTGTGTTGCCTGTGCCATGGGCACCTTCTCCGCAGGTCGT
GACGGTCACTGCAGACTTTGGACCAACTGTTCTCAGTTTGGATTTCTC
ACCATGTTCCCTGGGAACAAGACCCACAATGCTGTGTGCATCCCGGAG
CCACTGCCCACTGAGCAATACGGCCATTTGACTGTCATCTTCCTGGTC
ATGGCTGCATGCATTTTCTTCCTAACCACAGTCCAGCTCGGCCTGCAC
ATATGGCAGCTGAGGAGGCAACACATGTGTCCTCGAGAGACCCAGCCA
TTCGCGGAGGTGCAGTTGTCAGCTGAGGATGCTTGCAGCTTCCAGTTC
CCTGAGGAGGAACGCGGGGAGCAGACAGAAGAAAAGTGTCATCTGGGG
GGTCGGTGGCCATGA
Coding sequence of human GITR
(SEQ ID NO: 22)
ATGGCACAG CACGGGGCGA TGGGCGCGTT TCGGGCCCTG
TGCGGCCTGG CGCTGCTGTG CGCGCTCAGC CTGGGTCAGC
GCCCCACCGG GGGTCCCGGG TGCGGCCCTG GGCGCCTCCT
GCTTGGGACG GGAACGGACG CGCGCTGCTG CCGGGTTCAC
ACGACGCGCT GCTGCCGCGA TTACCCGGGC GAGGAGTGCT
GTTCCGAGTG GGACTGCATGTGTGTCCAGC CTGAATTCCA
CTGCGGAGAC CCTTGCTGCA CGACCTGCCG
GCACCACCCTTGTCCCCCAG GCCAGGGGGT ACAGTCCCAG
GGGAAATTCA GTTTTGGCTT CCAGTGTATCGACTGTGCCT
CGGGGACCTT CTCCGGGGGC CACGAAGGCC ACTGCAAACC
TTGGACAGAC TGCACCCAGT TCGGGTTTCT CACTGTGTTC
CCTGGGAACA AGACCCACAA CGCTGTGTGCGTCCCAGGGT
CCCCGCCGGC AGAGCCGCTT GGGTGGCTGA CCGTCGTCCT
CCTGGCCGTGGCCGCCTGCG TCCTCCTCCT GACCTCGGCC
CAGCTTGGAC TGCACATCTG GCAGCTGAGG AGTCAGTGCA
TGTGGCCCCG AGAGACCCAG CTGCTGCTGG AGGTGCCGCC
GTCGACCGAA GACGCCAGAA GCTGCCAGTT CCCCGAGGAA
GAGCGGGGCG AGCGATCGGC AGAGGAGAAGGGGCGGCTGG
GAGACCTGTG GGTGTGA

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

We claim:

1. A composition comprising:
an antibody, a ligand, or an antigen binding fragment thereof that specifically binds a co-stimulatory molecule; and
a nanoparticle comprising an mRNA encoding the co-stimulatory molecule.

2. The composition of claim 1, wherein the mRNA encoding the co-stimulatory molecule is encapsulated by the nanoparticle.

3. The composition of claim 1 or 2, wherein the nanoparticle comprises a phospholipid or a glycolipid.

4. The composition of claim 3, wherein the phospholipid is selected from the group consisting of PL1-PL18.

5. The composition of claim 4, wherein the phospholipid is PL1.

6. The composition of claim 3, wherein the glycolipid is selected from the group consisting of GL1-GL16.

7. The composition of claim 6, wherein the glycolipid is GL4.

8. The composition of any one of claims 1-7, wherein the co-stimulatory molecule is selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM3, TIM4, ICAM1, or LFA3.

9. The composition of claim 8, wherein the co-stimulatory molecule comprises OX40 or 4-1BB.

10. The composition of any one of claims 1-9, wherein the mRNA encoding the co-stimulatory molecule comprises a heterologous 5′ untranslated region (5′UTR).

11. The composition of any one of claims 1-9, wherein the mRNA encoding the co-stimulatory molecule comprises a heterologous 3′ untranslated region (3′UTR).

12. The composition of any one of claims 1-11, wherein the mRNA comprises a chemically modified nucleobase.

13. The composition of claim 12, wherein the chemically modified nucleobase is pseudouridine.

14. The composition of any one of claims 1-13, further comprising an immunotherapeutic agent.

15. The composition of claim 14, wherein the immunotherapeutic agent is selected from an anti-PDL1 antibody, an anti-PD1 antibody, an anti-CTLA4 antibody, or a combination thereof.

16. A pharmaceutical composition comprising a pharmaceutically acceptable carrier and an effective amount of the composition of any one of claims 1 to 15.

17. A method of stimulating a T cell comprising administering to a subject an effective amount of the composition of any one of claims 1 to 15 or the pharmaceutical composition of claim 16.

18. The method of claim 17, wherein the subject is a mammal.

19. The method of claim 18, wherein the mammal is a human.

20. A method of treating a cancer comprising administering to a subject in need thereof an effective amount of an antibody, a ligand, or an antigen binding fragment thereof that specifically binds a co-stimulatory molecule and a nanoparticle comprising an mRNA encoding the co-stimulatory molecule.

21. The method of claim 20, wherein the mRNA encoding the co-stimulatory molecule is encapsulated by the nanoparticle.

22. The method of claim 20 or 21, wherein the nanoparticle comprises a phospholipid or a glycolipid.

23. The method of claim 22, wherein the phospholipid is selected from the group consisting of PL1-PL18.

24. The method of claim 23, wherein the phospholipid is PL1.

25. The composition of claim 22, wherein the glycolipid is selected from the group consisting of GL1-GL16.

26. The composition of claim 25, wherein the glycolipid is GL4.

27. The method of any one of claims 20-26, wherein the co-stimulatory molecule is selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM3, TIM4, ICAM1, or LFA3.

28. The method of claim 27, wherein the co-stimulatory molecule comprises OX40 or 4-1BB.

29. The method of any one of claims 20 to 28, wherein the mRNA encoding the co-stimulatory molecule comprises a heterologous 5′ untranslated region (5′UTR).

30. The method of any one of claims 20 to 29, wherein the mRNA encoding the co-stimulatory molecule comprises a heterologous 3′ untranslated region (3′UTR).

31. The method of any one of claims 20 to 30, wherein the chemically modified nucleobase is pseudouridine.

32. The method of any one of claims 20 to 31, wherein the cancer comprises melanoma, colorectal cancer, lung cancer, colon cancer, or lymphoma.

33. The method of any one of claims 20 to 32, wherein the subject is a mammal.

34. The method of claim 33, wherein the mammal is a human.

35. The method of any one of claims 20-34, wherein the antibody or antigen binding fragment thereof and the nanoparticle are administered by intramuscular injection or systematically.

36. The method of any one of claims 20-35, further comprising administering an additional therapeutic agent.

37. The method of claim 36, wherein the additional therapeutic agent comprises an additional immunotherapeutic agent.

38. The method of claim 37, wherein the additional immunotherapeutic agent is selected from an anti-PDL1 antibody, an anti-PD1 antibody, an anti-CTLA4 antibody, or a combination thereof.

39. The method of any one of claims 20 to 38, where the antibody or antigen binding fragment thereof that specifically binds a co-stimulatory molecule and the nanoparticle comprising an mRNA encoding the co-stimulatory molecule are administered concurrently.
